(19) United States
(12) Patent Application Publication
(10) Pub. No.: US 2025/0346957 A1
(43) Pub. Date: Nov. 13, 2025

(54) METHODS AND COMPOSITIONS FOR CLASSIFYING AND TREATING LUNG CANCER

(71) Applicant: Genentech, Inc., South San Francisco, CA (US)

(72) Inventors: Habib HAMIDI, Fremont, CA (US); Barzin Y. NABET, Bala Cynwyd, PA (US); David Stuart SHAMES, San Francisco, CA (US)

(21) Appl. No.: 19/170,639

(22) Filed: Apr. 4, 2025

Related U.S. Application Data
(63) Continuation of application No. PCT/US2023/076126, filed on Oct. 5, 2023.
(60) Provisional application No. 63/378,477, filed on Oct. 5, 2022.

(51) Int. Cl. C12Q 1/6886; C12Q 1/6874

(52) U.S. Cl. CPC C12Q 1/6886; C12Q 1/6874; C12Q 2600/112

(57) ABSTRACT

The invention provides methods for classifying lung cancer (e.g., small cell lung cancer (SCLC), e.g., extensive stage SCLC (ES-SCLC)); methods for treating lung cancer in a patient, for example, by administering a treatment regimen that comprises a PD-1 axis binding antagonist (e.g., atezolizumab) to the patient. Also provided are compositions for use, kits, and articles of manufacture for use in classifying and treating lung cancer in a patient.

### SEQUENCE LISTING

The instant application contains a Sequence Listing which has been submitted electronically in XML format and is hereby incorporated by reference in its entirety. Said XML copy, created on Apr. 2, 2025, is named 50474-305003_Sequence_Listing_4_2_25 and is 10,409 bytes in size.

### FIELD OF THE INVENTION

This invention relates to methods and compositions for use in classifying and treating lung cancer (e.g., small cell lung cancer (SCLC)) in a patient.

### BACKGROUND OF THE INVENTION

Cancer remains one of the deadliest threats to human health. Cancers, or malignant tumors, metastasize and grow rapidly in an uncontrolled manner, making timely detection and treatment extremely difficult. In the U.S., cancer affects nearly 1.3 million new patients each year, and is the second leading cause of death after heart disease, accounting for approximately 1 in 4 deaths. Solid tumors are responsible for most of those deaths.

Small cell lung cancer (SCLC) is an aggressive neuroendocrine malignancy that accounts for approximately 15% of all lung cancers. There are two types of SCLC: limited-stage (LS)-SCLC and extensive-stage (ES)-SCLC, and at the time of diagnosis it is estimated that approximately 70% of patients have ES-SCLC. The long-term prognosis of patients with ES-SCLC is poor, and the relapse rate is high, with ˜75% of patients having locally advanced disease and over 90% of patients progressing within two years of treatment.

Thus, there is an unmet need in the field for improved diagnostic and therapeutic methods that identify patients likely to benefit from an anti-cancer therapy, e.g., treatment comprising a PD-1 axis binding antagonist.

### SUMMARY OF THE INVENTION

The present disclosure provides, inter alia, methods of classifying lung cancer (e.g., SCLC, e.g., ES-SCLC or LS-SCLC, including in the first-line (1L) treatment setting), methods of treating lung cancer, and related kits, compositions for use, uses, and systems (e.g., digital pathology systems). In one aspect, the invention features a method of classifying a small cell lung cancer (SCLC) in a human patient, the method comprising (a) assaying mRNA in a tumor sample from the patient to provide a transcriptional profile of the patient's tumor; and (b) assigning the patient's tumor sample into one of the following four subtypes based on the transcriptional profile of the patient's tumor: neuroendocrine inflamed (NE-I), neuroendocrine NEUROD-driven (NE-N), neuroendocrine achaete-scute homolog 1 (ASCL1)-driven (NE-A), or non-neuroendocrine inflamed (nNE-I), thereby classifying the SCLC in the patient.

In some aspects, step (b) comprises assigning the patient's tumor sample into one of the following four subtypes using a machine learning classifier based on the transcriptional profile of the patient's tumor: NE-I, NE-N, NE-A, or nNE-I.

In another aspect, the invention features a method of treating an SCLC in a human patient, the method comprising: classifying the SCLC in the patient according to any one of the methods disclosed herein; and administering an anti-cancer therapy to the patient based on the SCLC subtype. In one aspect, the anti-cancer therapy comprises atezolizumab.

In another aspect, the invention features an anti-cancer therapy for use in treating an SCLC in a human patient, wherein the SCLC in the patient has been classified according to any one of the methods disclosed herein. In one aspect, the anti-cancer therapy comprises atezolizumab.

In another aspect, the invention features the use of an anti-cancer therapy in the preparation of a medicament for treating an SCLC in a human patient, wherein the SCLC in the patient has been classified according to any one of the methods disclosed herein. In one aspect, the anti-cancer therapy comprises atezolizumab.

In another aspect, the invention features a method of identifying a patient having an SCLC who is likely to benefit from an anti-cancer therapy comprising atezolizumab, the method comprising: determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising atezolizumab.

In another aspect, the invention features a method of selecting a therapy for a patient having an SCLC, the method comprising: (a) determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising atezolizumab; and (b) selecting an anti-cancer therapy comprising atezolizumab for the patient identified as one who is likely to benefit from the anti-cancer therapy.

In another aspect, the invention features a method of treating a patient having an SCLC, the method comprising: (a) determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising atezolizumab; and (b) administering an anti-cancer therapy comprising atezolizumab to the patient identified as one who is likely to benefit from the anti-cancer therapy.

In another aspect, the invention features a method of treating a patient having an SCLC, the method comprising administering an anti-cancer therapy comprising atezolizumab to the patient, wherein the patient has been determined to have an increased expression level, relative to a reference expression level, of a T-eff signature comprising CD8A, GZBA, GZMB, PRF1, IFNG, CXCL9, CXCL10, and TBX21 and a decreased expression level, relative to a reference expression level, of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient.

In some aspects, the anti-cancer therapy includes a PD-1 axis binding antagonist (e.g., an anti-PD-L1 antibody, e.g., atezolizumab). In some aspects, the anti-cancer therapy includes atezolizumab. In some aspects, the anti-cancer therapy includes a CTLA4 antagonist (e.g., an anti-CTLA4 antibody). In some aspects, the anti-cancer therapy comprising a PD-1 axis binding antagonist (e.g., atezolizumab) further comprises carboplatin and etoposide. In some aspects, the anti-cancer therapy includes a PD-1 axis binding antagonist (e.g., atezolizumab) and a DNA damage response (DDR)-targeting agent (e.g., an anti-delta-like ligand 3 (DLL3) antibody-drug conjugate (ADC) or an anti-DLL3 bispecific T cell engager (BiTE)). In some aspects, the anti-cancer therapy includes a PD-1 axis binding antagonist (e.g., atezolizumab) and a myeloid repolarization agent (e.g., a Toll-like receptor 7 (TLR7) agonist). In some aspects, the anti-cancer therapy includes a PD-1 axis binding antagonist (e.g., atezolizumab) and one or more additional agents (e.g., a REST-targeted therapy). In some aspects, the anti-cancer therapy includes a PD-1 axis binding antagonist (e.g., atezolizumab) and one or more additional agents (e.g., a chemotherapeutic agent, or a combination thereof).

In another aspect, the invention features a kit for performing any one of the methods disclosed herein. In some aspects, the kit comprises (a) reagents for assaying mRNA in a tumor sample from the patient to provide a transcriptional profile of the patient's tumor; and (b) instructions for assigning the patient's tumor sample into following four subtypes based on the transcriptional profile of the patient's tumor: NE-I, NE-N, NE-A, or nNE-1, thereby classifying the SCLC.

### BRIEF DESCRIPTION OF THE DRAWINGS

FIG. 1 depicts a system diagram illustrating an example of a digital pathology system, in accordance with some example embodiments.

FIG. 2 depicts a flowchart illustrating an example of a process for image-based SCLC molecular subtype classification, in accordance with some example embodiments.

FIG. 3 depicts a block diagram illustrating an example of a computing system, in accordance with some example embodiments.

FIG. 4A is a series of graphs showing a cophenetic correlation for an increasing number of non-negative matrix factorization (NMF)-defined clusters (top) and a consensus matrix for the optimal number of clusters (k=4) (bottom). In FIG. 4A, NMF clusters NMF1, NMF2, NMF4, and NMF3 are shown from left to right.

FIG. 4B is a pie chart showing the relative proportion of patient tumors by NMF-identified subtype in IMpower133.

FIG. 4C is a heatmap showing hierarchical clustering within each NMF subtype of genes that have been previously described to define small cell lung cancer (SCLC) subtypes. Z scores are indicated for each gene. Each column represents one patient tumor. NE-N, neuroendocrine NEUROD1-driven; NE-A, neuroendocrine ASCL1-driven; NE-I, neuroendocrine inflamed; nNE-I, nonneuroendocrine inflamed.

FIG. 4D is a bar plot showing the fraction of patients within each NMF subtype showing previously identified transcription factor subtypes (TF Subtypes; Rudin et al. Nat Rev Cancer. 19:289-297 (2019)) and unbiased clustering from limited-stage (LS)-SCLC RNA-seq (MDACC Subtypes; Gay et al. Cancer Cell. 39:346-360.e7 (2021)).

FIG. 4E is an alluvial plot showing the number of patients with overlap in molecular subtype assignment using the various methods. TF, TF Subtypes; GNE, present study from Genentech, Inc.; MDACC, MD Anderson Cancer Center Subtypes.

FIG. 5A is a series of box plots showing the expression of key transcription factors in each SCLC molecular subtype. log 2(TPM+1), transcript-per-million (TPM) plus 1 normalization and subsequent log 2-transformation.

FIG. 5B is a heatmap showing mean-scaled expression of hallmark gene signatures that are significantly different in one of the molecular subtypes.

FIG. 5C is an oncoprint displaying somatic alterations in each SCLC molecular subtype. Each column represents a patient with paired whole-exome sequencing (WES) and RNA-seq. The heatmap at the bottom shows the log 2 ratio of tumor to germline copy number (CN log R) variation for TP53 and RB1. The horizontal bar plots to the right represent the number of patients with alterations for each gene. The percentages on the y-axis indicate the proportion of patients with the somatic alteration.

FIG. 6A is a heatmap showing gene expression in each SCLC molecular subtype related to T-effector signaling (tGE8), immune stimulatory molecules (Stim.), immune inhibitory molecules (Inhibitory), myeloid cells (Myeloid), and angiogenesis (Angio.).

FIG. 6B is a heatmap showing gene expression in each SCLC molecular subtype for tumor microenvironment (TME)-related gene signature expression.

FIG. 6C is a bar plot showing the fraction of patients in each SCLC molecular subtype and the percentage of immune cells expressing PD-L1 by immunohistochemistry (IHC) using the SP263 assay.

FIG. 7A is a bar plot showing a summary of the defining features of each SCLC molecular subtype. Each color (i.e., dark gray, light gray, gray) indicates a set of molecular features within each subtype.

FIG. 7B is a series of bar plots showing the fraction of patients with objective responses (light gray) or stable or progressive disease (dark gray) in each arm of IMpower133 in the RNA-seq biomarker evaluable population (BEP) and within each subtype. Atezo, atezolizumab in combination with carboplatin and etoposide (CE); placebo, placebo plus CE.

FIG. 7C is a forest plot showing the progression-free survival (PFS) hazard ratio (HR) comparing atezolizumab plus CE (Atezo) versus placebo plus CE (placebo) in the intent-to-treat population (ITT), BEP, and each subtype. Shown on the right is the median PFS in months (mo.) for each subgroup.

FIG. 7D is a forest plot showing the overall survival (OS) HR comparing atezolizumab plus CE (Atezo) versus placebo plus CE (placebo) in the ITT population, BEP, and each subtype. Shown on the right is the median OS in months (mo.) for each subgroup.

FIGS. 7E and 7F is a series of Kaplan-Meier plots showing OS in patients treated with atezolizumab plus CE (Atezo) versus placebo plus CE (Placebo) in the BEP (FIG. 7E) and within the NE-I molecular subtype (FIG. 7F). Dark gray, atezo; light gray, placebo.

FIG. 8A is a volcano plot showing differentially expressed genes comparing the nNE-I (left) and NE-I (right) molecular subtypes. The labeled data points indicate genes related to cell types in the lung, as well as the neuroendocrine (NE) and non-neuroendocrine (nonNE) subtypes, and are classified in the legend on the right.

FIG. 8B is a series of box plots showing the gene expression of lung cell type specific signatures derived from normal lung single cell RNA-seq (scRNA-seq) data in tumors of the nNE-I and NE-I subtypes.

FIG. 8C is a bar plot showing gene set enrichment analysis of immune-related gene signatures comparing the nNE-I (left) and NE-I (right) subtypes.

FIG. 8D is a bar plot showing the fraction of patients in each subtype and the overlap between T-effector (tGE8) and tumor-associated macrophage (TAM) signature high (hi) and low (lo) groups. The groups were defined by cohort-wide median split for each signature.

FIG. 8E is a forest plot showing the OS HR comparing atezolizumab plus CE (atezo) versus placebo plus CE (placebo) in the BEP, tGE^{hi}, tGE^{hi}, TAM^{hi}, TAM^{lo}, tGE8^{hi}/TAM^{hi}, and tGE8^{hi}/TAM^{lo} groups.

FIG. 8F is a Kaplan-Meier plot showing OS in patients treated with atezolizumab plus CE (atezo) versus placebo plus CE (placebo) with high T-effector signature score (>median) and low TAM signature score (≤median) (tGE8^{hi}/TAM^{lo}). Dark gray, atezo; light gray, placebo.

FIG. 9A is a correlation matrix of SCLC-related genes and TAM signature in the subset of tumors with high T-eff signature score (>median).

FIG. 9B is a volcano plot depicting differentially expressed genes between samples with high TAM-signature scores versus low TAM-signature scores within the T-eff high tumors in an independently procured SCLC bulk RNA-seq dataset. The labeled data points indicate genes related to cell types, as well as the NE and nonNE subtypes, and are classified in the legend on the right.

FIG. 9C is a series of box plots showing REST (left) and MYC (right) gene expression in TAM-high versus TAM-low tumors within T-eff high tumors in the independent SCLC dataset.

FIG. 9D is a box plot showing REST gene expression in TAM-high versus TAM-low tumors within T-eff high tumors from George et al. Nature. 524:47-53 (2015).

FIG. 9E is a heatmap showing expression of immunomodulatory genes after Rest overexpression in mouse SCLC cell lines (data from Shue et al. Nat Commun. 13:2690 (2022)).

FIG. 10A is a box plot showing blood tumor mutational burden score distribution among SCLC molecular subtypes.

FIG. 10B is a graph showing negative log 10 adjusted P-value for a chi-square test performed for each recurrently mutated gene to assess significant differences between the subtypes. The gray-dashed line is equivalent to an adjusted P-value of 0.05.

FIG. 10C is a box plot showing the NOTCH hallmark gene signature score in samples without (WT) or with (MUT) impactful NOTCH family mutations (NOTCH3 status).

FIG. 11 is a heatmap showing relative gene expression of antigen presentation machinery genes in each SCLC molecular subtype.

FIGS. 12A and 12B is a series of graphs showing PFS (FIG. 12A) and OS (FIG. 12B) HR comparing atezolizumab plus CE versus placebo plus CE in the ITT, BEP, and each subtype with the patients previously classified as SCLC-P (Gay et al. Cancer Cell. 39:346-360.e7 (2021)) removed.

FIG. 13A is a bar plot showing the fraction of patients in each subtype established utilizing unbiased clustering from LS-SCLC RNA-seq (MDACC subtypes) (Gay et al. Cancer Cell. 39:346-360.e7 (2021)).

FIG. 13B is a schematic diagram showing heterogeneity of immune infiltrated SCLC tumors within previously reported subtypes (SCLC-A, SCLC-N, SCLC-P, and SCLC-I). Immune infiltrated tumors in each previously reported subtype are classified as SCLC-I-NE or SCLC-I-nNE.

### DETAILED DESCRIPTION OF THE INVENTION

The present invention provides diagnostic and therapeutic methods and compositions for cancer, for example, lung cancer (e.g., SCLC, e.g., ES-SCLC or LS-SCLC, including in the first-line (1L) treatment setting). The invention is based, at least in part, on the discovery that the methods of classification described herein identify patient subgroups that have unexpectedly favorable response to anti-cancer therapies, including anti-cancer therapies that include a PD-1 axis binding antagonist (e.g., an anti-PD-L1 antibody, e.g., atezolizumab), as shown in Example 1. Moreover, Example 1 demonstrates that the methods of classification herein are expected to be effective for identifying patient subgroups for a PD-1 axis binding antagonist (e.g., an anti-PD-L1 antibody, e.g., atezolizumab) in combination with other anti-cancer therapies, such carboplatin and etoposide. Based on these data, it is expected that the methods of classification described herein can also identify patient subgroups with favorable response to a PD-1 axis binding antagonist (e.g., an anti-PD-L1 antibody, e.g., atezolizumab), alone or in combination with other anti-cancer therapies.

### I. Definitions

The term “anti-cancer therapy” refers to a therapy useful in treating cancer. An anti-cancer therapy may include a treatment regimen with one or more anti-cancer therapeutic agents. Examples of anti-cancer therapeutic agents include, but are limited to, an immunotherapy agent (e.g., a PD-1 axis binding antagonist), a cytotoxic agent, a chemotherapeutic agent (e.g., a platinum-based chemotherapeutic agent (e.g., carboplatin) and/or a topoisomerase inhibitor (e.g., etoposide)), a growth inhibitory agent, a stromal inhibitor, a metabolism inhibitor, a complement antagonist, a radiation therapy agent, an anti-angiogenic agent, an antibody-drug conjugate (ADC), and other agents to treat cancer. Combinations thereof are also included in the invention.

An “immunoconjugate” or “antibody drug conjugate” or “ADC” is an antibody conjugated to one or more heterologous molecule(s), including but not limited to a cytotoxic agent. Exemplary, non-limiting antibody drug conjugates include anti-HER2 antibody drug conjugates (anti-HER2 ADC) (e.g., trastuzumab emtansine (T-DM1, ado-trastuzumab emtansine, KADCYLA®, Genentech), trastuzumab deruxtecan (DS-8201a, T-DXd, ENHERTU®, Gilead), trastuzumab duocarmazine (SYD985, Byondis), A166, XMT-1522, MEDI-4276, ARX788, RC48-ADC, BAT8001, PF-06804103) and anti-TROP2 antibody drug conjugates (anti-TROP2 ADC) (e.g., sacituzumab govitecan (TRODELVY®, Gilead), datopotamab deruxtecan (Dato-DXd, DS-1062a, Daiichi Sankyo, AstraZeneca), BAT8003 (Biothera)). Exemplary, non-limiting antibody drug conjugates are described in Criscitiello et al. J Hematol Oncol. 14:20 (2021).

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

A “stromal inhibitor” refers to any molecule that partially or fully blocks, inhibits, or neutralizes a biological activity and/or function of a gene or gene product associated with stroma (e.g., tumor-associated stroma). In some embodiments, the stromal inhibitor partially or fully blocks, inhibits, or neutralizes a biological activity and/or function of a gene or gene product associated with fibrotic tumors. In some embodiments, treatment with a stromal inhibitor results in the reduction of stroma, thereby resulting in an increased activity of an immunotherapy; for example, by increasing the ability of activating immune cells (e.g., proinflammatory cells) to infiltrate a fibrotic tissue (e.g., a fibrotic tumor). Targets for stromal gene antagonists are known in the art; for example, see Turley et al., Nature Reviews Immunology 15:669-682, 2015 and Rosenbloom et al., Biochimica et Biophysica Acta 1832:1088-1103, 2013. In some embodiments, the stromal inhibitor is a transforming growth factor beta (TGF-β), podoplanin (PDPN), leukocyte-associated immunoglobulin-like receptor 1 (LAIR1), SMAD, anaplastic lymphoma kinase (ALK), connective tissue growth factor (CTGF/CCN2), endothelial-1 (ET-1), AP-1, interleukin (IL)-13, lysyl oxidase homolog 2 (LOXL2), endoglin (CD105), fibroblast activation protein (FAP), vascular cell adhesion protein 1 (CD106), thymocyte antigen 1 (THY1), beta 1 integrin (CD29), platelet-derived growth factor (PDGF), PDGF receptor A (PDGFRα), PDGF receptor B (PDGFRβ), vimentin, smooth muscle actin alpha (ACTA2), desmin, endosialin (CD248), or S100 calcium-binding protein A4 (S100A4) antagonist.

A “TGF-β antagonist” or a “TGF-β inhibitor,” as used interchangeably herein, refers to any molecule that decreases, blocks, inhibits, abrogates or interferes with signal transduction resulting from the interaction of TGF-β with one or more of its interaction partners, such as a TGF-β cellular receptor. In some embodiments, a “TGF-β binding antagonist” is a molecule that inhibits the binding of TGF-β to its binding partners. In some embodiments, the TGF-β antagonist inhibits the activation of TGF-β. In some embodiments, the TGF-β antagonist includes an anti-TGF-β antibody, antigen binding fragments thereof, an immunoadhesin, a fusion protein, an oligopeptide, and other molecules that decrease, block, inhibit, abrogate or interfere with signal transduction resulting from the interaction of TGF-β with one or more of its interaction partners. In some embodiments, the TGF-β antagonist is a polypeptide, a small molecule, or a nucleic acid. In some embodiments, the TGF-β antagonist (e.g., the TGF-β binding antagonist) inhibits TGF-β1, TGF-β2, and/or TGF-β3. In some embodiments, the TGF-β antagonist (e.g., the TGF-β binding antagonist) inhibits TGF-β receptor-1 (TGFBR1), TGF-β receptor-2 (TGFBR2), and/or TGF-β receptor-3 (TGFBR3).

The terms “anti-TGF-β antibody” and “an antibody that binds to TGF-β” refer to an antibody that is capable of binding TGF-β with sufficient affinity such that the antibody is useful as a diagnostic and/or therapeutic agent in targeting TGF-β. In one embodiment, the extent of binding of an anti-TGF-β antibody to an unrelated, non-TGF-β protein is less than about 10% of the binding of the antibody to TGF-β as measured, for example, by a radioimmunoassay (RIA). In certain embodiments, an anti-TGF-β antibody binds to an epitope of TGF-β that is conserved among TGF-β from different species. In some embodiments, the anti-TGF-β antibody inhibits TGF-β1, TGF-β2, and/or TGF-β3. In some embodiments, the anti-TGF-β antibody inhibits TGF-β1, TGF-β2, and TGF-β3. In some embodiments, the anti-TGF-β antibody is a pan-specific anti-TGF-β antibody. In some embodiments, the anti-TGF-β antibody may be any anti-TGF-β antibody disclosed in, for example, U.S. Pat. No. 5,571,714 or in International Patent Application Nos. WO 92/00330, WO 92/08480, WO 95/26203, WO 97/13844, WO 00/066631, WO 05/097832, WO 06/086469, WO 05/010049, WO 06/116002, WO 07/076391, WO 12/167143, WO 13/134365, WO 14/164709, or WO 16/201282, each of which is incorporated herein by reference in its entirety. In particular embodiments, the anti-TGF-β antibody is fresolimumab, metelimumab, lerdelimumab, 1D11, 2G7, or a derivative thereof.

A “metabolism inhibitor” refers to any molecule that disrupts metabolism (e.g., basal metabolism), metabolic pathways and/or levels of metabolites of a cell (e.g., a cancer cell), either directly or indirectly. In some embodiments, a metabolism inhibitor may stimulate any change in metabolism (e.g., basal metabolism), metabolic pathways, and/or levels of metabolites of a cell. Metabolic pathways can include, but are not limited to, amino acid catabolismcellular respiration, oxidative phosphorylation (OXPHOS), glycolysis, fatty acid oxidation, fatty acid metabolism, electron transport chain (ETC) complex I activity, ETC complex II activity, ETC complex III activity, ETC complex IV activity, the tricarboxylic acid (TCA) cycle, amino acid uptake, any catabolic pathway, any anabolic pathway, any amphibolic pathway, catabolismanabolism, gluconeogenesis, glycogenolysis, glycogenesis, the urea cycle, aminotransferase pathways, acetyl-CoA synthesis pathways, pentose phosphate pathway, fructolysis, galactolysis, glycosylation, beta oxidation, fatty acid degradation, fatty acid synthesis, steroid metabolism, sphingolipid metabolism, eicosanoid metabolism, ketosis, reverse cholesterol transport, glutamine/glutamate catabolismasparagine/aspartate catabolismalanine catabolismarginine, ornithine and proline catabolismserine catabolismthreonine catabolism glycine catabolismcysteine catabolismo methionine catabolismleucine, isoleucine and valine catabolismphenylalanine and tyrosine catabolismo lysine catabolismhistidine catabolismtryptophan catabolismor any combination thereof. In some embodiments, the metabolism inhibitor is a proprotein convertase subtilisin/kexin type 9 serine protease (PCSK9) inhibitor (e.g., an anti-PCSK9 antibody, e.g., alirocumab or evolocumab), fatty acid synthase (FAS) inhibitor (e.g., cerulenin, C75, isoniazid, or orlistat (tetrahydrolipstatin)), carnitine palmitoyltransferase-1 (CPT-1) inhibitor (e.g., etomoxir), GLUT4 inhibitor (e.g., ritonavir, indinavir, or analogs or derivatives thereof), or OXPHOS inhibitor (e.g., compounds within the biguanide class of drugs, e.g., metformin, phenformin, buformin, and pharmaceutically acceptable salts thereof).

An “angiogenesis inhibitor” or “anti-angiogenic agent” or “anti-angiogenesis agent,” as used interchangeably herein, refers to a small molecular weight substance (including tyrosine kinase inhibitors), a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (imatinib mesylate). Anti-angiogenesis agents also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5 (12): 1359-1364 (1999); Tonini et al., Oncogene, 22:6549-6556 (2003) and Sato Int. J. Clin. Oncol., 8:200-206 (2003). In some examples, the angiogenesis inhibitor is an anti-VEGF antibody or an antigen-binding fragment thereof, e.g., bevacizumab.

A “DNA damage response (DDR)-targeting agent” or “DDR-targeting agent” refers to any therapeutic agent that induces the DNA damage response of a cell (e.g., a cancer cell), either directly or indirectly. Exemplary, non-limiting DDR-targeting agents include an anti-delta-like ligand 3 (DLL3) antibody-drug conjugate (ADC) (e.g., Rova-T) or an anti-DLL3 bispecific T cell engager (BiTE) (e.g., AMG 757).

The term “immunotherapy agent” refers the use of a therapeutic agent that modulates an immune response. Exemplary, non-limiting immunotherapy agents include a PD-1 axis binding antagonist, a CTLA-4 antagonist (e.g., an anti-CTLA-4 antibody (e.g., ipilimumab)), a TIGIT antagonist (e.g., an anti-TIGIT antibody (e.g., tiragolumab)), PD1-IL2v (a fusion of an anti-PD-1 antibody and modified IL-2), PD1-LAG3, IL-15, anti-CCR8 (e.g., an anti-CCR8 antibody, e.g., FPA157), FAP-4-1BBL (fibroblast activation protein-targeted 4-1BBL agonist), or a combination thereof. In some examples, the immunotherapy agent is an immune checkpoint inhibitor. In some examples, the immunotherapy agent is a CD28, OX40, GITR, CD137, CD27, ICOS, HVEM, NKG2D, MICA, or 2B4 agonist or a CTLA-4, PD-1 axis, TIM-3, BTLA, VISTA, LAG-3, B7H4, CD96, TIGIT, or CD226 antagonist. Other particular immunotherapy agents include anti-TIGIT antibodies (e.g., tiragolumab) and antigen-binding fragments thereof, anti-CTLA-4 antibodies or antigen-binding fragments thereof, anti-CD27 antibodies or antigen-binding fragments thereof, anti-CD30 antibodies or antigen-binding fragments thereof, anti-CD40 antibodies or antigen-binding fragments thereof, anti-4-1BB antibodies or antigen-binding fragments thereof, anti-GITR antibodies or antigen-binding fragments thereof, anti-OX40 antibodies or antigen-binding fragments thereof, anti-TRAILR1 antibodies or antigen-binding fragments thereof, anti-TRAILR2 antibodies or antigen-binding fragments thereof, anti-TWEAK antibodies or antigen-binding fragments thereof, anti-TWEAKR antibodies or antigen-binding fragments thereof, anti-BRAF antibodies or antigen-binding fragments thereof, anti-MEK antibodies or antigen-binding fragments thereof, anti-CD33 antibodies or antigen-binding fragments thereof, anti-CD20 antibodies or antigen-binding fragments thereof, anti-CD52 antibodies or antigen-binding fragments thereof, anti-A33 antibodies or antigen-binding fragments thereof, anti-GD3 antibodies or antigen-binding fragments thereof, anti-PSMA antibodies or antigen-binding fragments thereof, anti-Ceacan 1 antibodies or antigen-binding fragments thereof, anti-Galedin 9 antibodies or antigen-binding fragments thereof, anti-HVEM antibodies or antigen-binding fragments thereof, anti-VISTA antibodies or antigen-binding fragments thereof, anti-B7 H4 antibodies or antigen-binding fragments thereof, anti-HHLA2 antibodies or antigen-binding fragments thereof, anti-CD155 antibodies or antigen-binding fragments thereof, anti-CD80 antibodies or antigen-binding fragments thereof, anti-BTLA antibodies or antigen-binding fragments thereof, anti-CD160 antibodies or antigen-binding fragments thereof, anti-CD28 antibodies or antigen-binding fragments thereof, anti-CD226 antibodies or antigen-binding fragments thereof, anti-CEACAM1 antibodies or antigen-binding fragments thereof, anti-TIM3 antibodies or antigen-binding fragments thereof, anti-CD96 antibodies or antigen-binding fragments thereof, anti-CD70 antibodies or antigen-binding fragments thereof, anti-CD27 antibodies or antigen-binding fragments thereof, anti-LIGHT antibodies or antigen-binding fragments thereof, anti-CD137 antibodies or antigen-binding fragments thereof, anti-DR4 antibodies or antigen-binding fragments thereof, anti-CR5 antibodies or antigen-binding fragments thereof, anti-FAS antibodies or antigen-binding fragments thereof, anti-CD95 antibodies or antigen-binding fragments thereof, anti-TRAIL antibodies or antigen-binding fragments thereof, anti-DR6 antibodies or antigen-binding fragments thereof, anti-EDAR antibodies or antigen-binding fragments thereof, anti-NGFR antibodies or antigen-binding fragments thereof, anti-OPG antibodies or antigen-binding fragments thereof, anti-RANKL antibodies or antigen-binding fragments thereof, anti-LTBR antibodies or antigen-binding fragments thereof, anti-BCMA antibodies or antigen-binding fragments thereof, anti-TACI antibodies or antigen-binding fragments thereof, anti-BAFFR antibodies or antigen-binding fragments thereof, anti-EDAR2 antibodies or antigen-binding fragments thereof, anti-TROY antibodies or antigen-binding fragments thereof, and anti-RELT antibodies or antigen-binding fragments thereof.

The terms “programmed death ligand 1” and “PD-L1” refer herein to native sequence human PD-L1 polypeptide. Native sequence PD-L1 polypeptides are provided under UniProt Accession No. Q9NZQ7. For example, the native sequence PD-L1 may have the amino acid sequence as set forth in UniProt Accession No. Q9NZQ7-1 (isoform 1). In another example, the native sequence PD-L1 may have the amino acid sequence as set forth in UniProt Accession No. Q9NZQ7-2 (isoform 2). In yet another example, the native sequence PD-L1 may have the amino acid sequence as set forth in UniProt Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In some embodiments, the cancer is a lung cancer. In some embodiments, the lung cancer is an SCLC (e.g., ES-SCLC or LS-SCLC). The cancer may be locally advanced or metastatic. In some instances, the cancer is locally advanced. In other instances, the cancer is metastatic. In some instances, the cancer may be unresectable (e.g., unresectable locally advanced or metastatic cancer).

As used herein, “cluster” or “subtype,” as used interchangeably herein, refers to a subtype of a cancer (e.g., lung cancer (e.g., SCLC, e.g., ES-SCLC or LS-SCLC)) that is defined, e.g., transcriptionally (e.g., as assessed by RNA-seq or other techniques described herein) and/or by evaluation of somatic alterations. Cluster analysis can be used to identify subtypes of cancer by clustering samples (e.g., tumor samples) from patients having similar gene expression patterns and to find groups of genes that have similar expression profiles across different samples. A patient's sample (e.g., tumor sample) can be assigned into a cluster as described herein. In some examples, clusters are identified by non-negative matrix factorization (NMF); however, other clustering approaches are described herein and known in the art. In some examples, a patient's tumor sample is assigned into one of the following four subtypes based on the transcriptional profile of the patient's tumor: neuroendocrine inflamed (NE-I), neuroendocrine NEUROD-driven (NE-N), neuroendocrine achaete-scute homolog 1 (ASCL1)-driven (NE-A), or non-neuroendocrine inflamed (nNE-I). A patient's tumor sample may be assigned into a cluster as described herein using methods described herein, e.g., using a classifier as described herein (e.g., the set of genes set forth in Table 1 or a subset thereof).

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and one or more additional therapeutic agents). Treating herein includes, inter alia, adjuvant therapy, neoadjuvant therapy, non-metastatic cancer therapy (e.g., locally advanced cancer therapy), and metastatic cancer therapy. The treatment may be first-line (also referred to as “1L”) treatment (e.g., the patient may be previously untreated or not have received prior systemic therapy), second-line (also referred to as “2L”), or later (2L+) treatment (e.g., third-line or fourth-line treatment). In some examples, the treatment may be first-line treatment (e.g., the patient may be previously untreated or not have received prior systemic therapy). In some examples, the patient is chemotherapy naïve. In some examples, the treatment may be 2L or later (2L+) treatment. In some examples, the treatment is adjuvant therapy. In other examples, the treatment is neoadjuvant therapy.

Herein, an “effective amount” refers to the amount of a therapeutic agent (e.g., a PD-1 axis binding antagonist (e.g., atezolizumab) or a combination of therapeutic agents (e.g., a PD-1 axis antagonist and one or more additional therapeutic agents), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), progression-free survival (PFS) and/or overall survival (OS)), and/or improved duration of response (DOR). Improvement (e.g., in terms of response rate (e.g., ORR, CR, and/or PR), survival (e.g., PFS and/or OS), or DOR) may be relative to a suitable reference, for example, observation or a reference treatment (e.g., treatment that does not include the PD-1 axis binding antagonist (e.g., treatment with placebo)). In some instances, improvement (e.g., in terms of response rate (e.g., ORR, CR, and/or PR), survival (e.g., DFS, DSS, distant metastasis-free survival, PFS, and/or OS), DOR, and/or improved time to deterioration of function and QoL) may be relative to observation. In some instances, treatment with an anti-cancer therapy that includes atezolizumab may be compared with a reference treatment which is treatment with chemotherapy (e.g., carboplatin and/or etoposide).

As used herein, “complete response” and “CR” refers to disappearance of the cancer. In some examples, tumor response is assessed according to RECIST v1.1. For example, CR may be the disappearance of all target lesions and non-target lesions and (if applicable) normalization of tumor marker level or reduction in short axis of any pathological lymph nodes to <10 mm.

As used herein, “partial response” and “PR” refers to at least a 30% decrease in the sum of the longest diameters (SLD) of target lesions, taking as reference the baseline SLD prior to treatment. In some examples, tumor response is assessed according to RECIST v1.1. For example, PR may be a ≥30% decrease in the sum of diameters (SoD) of target lesions (taking as reference the baseline SoD) or persistence of ≥1 non-target lesions(s) and/or (if applicable) maintenance of tumor marker level above the normal limits. In some examples, the SoD may be of the longest diameters for non-nodal lesions, and the short axis for nodal lesions.

As used herein, “disease progression,” “progressive disease,” and “PD” refers to an increase in the size or number of target lesions. For example, PD may be a ≥20% relative increase in the sum of diameters (SoD) of all target lesions, taking as reference the smallest SoD on study, including baseline, and an absolute increase of ≥5 mm; ≥1 new lesion(s); and/or unequivocal progression of existing non-target lesions. In some examples, the SoD may be of the longest diameters for non-nodal lesions, and the short axis for nodal lesions.

As used herein, “overall response rate,” “objective response rate,” and “ORR” refer interchangeably to the sum of CR rate and PR rate. For example, ORR may refer to the percentage of participants with a documented CR or PR.

As used herein, “progression-free survival” and “PFS” refer to the length of time during and after treatment during which the cancer does not get worse. PFS may include the amount of time patients have experienced a CR or a PR, as well as the amount of time patients have experienced stable disease. For example, PFS may be the time from randomization to PD, as determined by the investigator per RECIST v1.1, or death from any cause, whichever occurred first. In one example, progression is defined using RECIST v1.0, as at least 20% increase in the sum of the longest diameter of target lesions compared to baseline, or unequivocal progression in non-target lesion(s), or the appearance of new lesion(s).

As used herein, “overall survival” and “OS” refer to the length of time from either the date of diagnosis or the start of treatment for a disease (e.g., cancer) that the patient is still alive. For example, OS may be the time from randomization to death due to any cause.

As used herein, the term “duration of response” and “DOR” refer to a length of time from documentation of a tumor response until disease progression or death from any cause, whichever occurs first. For example, DOR may be the time from the first occurrence of CR/PR to PD as determined by the investigator per RECIST v1.1, or death from any cause, whichever occurred first.

As used herein, the term “chemotherapeutic agent” refers to a compound useful in the treatment of cancer, such as lung cancer (e.g., SCLC, e.g., ES-SCLC or LS-SCLC). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl) methoxy]phenyl]-6 [5 [[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin w1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), JAVLOR® (vinflunine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “small molecule” refers to any molecule with a molecular weight of about 2000 daltons or less, preferably of about 500 daltons or less. In some instances, a small molecule is any molecule with a molecular weight of 2000 daltons or less, preferably of 500 daltons or less.

The term “patient” refers to a human patient. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)). Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and one or more chemotherapeutic agents (e.g., a platinum-based chemotherapeutic agent (e.g., carboplatin) and/or a topoisomerase inhibitor (e.g., etoposide)). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle.

The term “detection” includes any means of detecting, including direct and indirect detection.

The term “biomarker” as used herein refers to an indicator, e.g., predictive, diagnostic, and/or prognostic, which can be detected in a sample, for example, a cluster, gene (e.g., PD-L1), an alteration (e.g., a somatic alteration), or ctDNA disclosed herein. The biomarker may serve as an indicator of a particular subtype of a disease or disorder (e.g., cancer) characterized by certain, molecular, pathological, histological, and/or clinical features. Biomarkers include, but are not limited to, clusters, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. In some examples, a biomarker is a cluster, e.g., a cluster identified by NMF, e.g., one of the following subtypes: NE-I, NE-N, NE-A, or nNE-I. In other examples, a biomarker is a gene. In yet other examples, a biomarker is an alteration (e.g., a somatic alteration).

The presence and/or expression level/amount of various biomarkers described herein in a sample can be analyzed by any suitable methodologies, including, but not limited to, immunohistochemistry (“IHC”), Western blot analysis, immunoprecipitation, molecular binding assays, ELISA, ELIFA, flow cytometry, fluorescence activated cell sorting (“FACS”), MASSARRAY®, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, massively parallel DNA sequencing (e.g., next-generation sequencing), NANOSTRING®, polymerase chain reaction (PCR), including quantitative real time PCR (qRT-PCR) and reverse transcription-quantitative polymerase chain reaction (RT-qPCR), and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used. The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “diagnosis” is used herein to refer to the identification or classification of a molecular or pathological state, disease or condition (e.g., cancer (e.g., lung cancer (e.g., SCLC, e.g., ES-SCLC or LS-SCLC))). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)). In some examples, a patient may be diagnosed by classifying the patient's cancer according to the methods disclosed herein, e.g., by assigning the patient's tumor sample into one of the following four subtypes based on the transcriptional profile of the patient's tumor: NE-I, NE-N, NE-A, or nNE-I.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a (lung cancer (e.g., SCLC, e.g., ES-SCLC or LS-SCLC) tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” “control tissue,” or “reference level,” as used herein, refers to a sample, cell, tissue, standard, or level that is used for comparison purposes. In one embodiment, a reference sample, reference cell, reference tissue, control sample, control cell, control tissue, or reference level is obtained from a healthy and/or non-diseased part of the body (e.g., tissue or cells) of the same patient. For example, the reference sample, reference cell, reference tissue, control sample, control cell, control tissue, or reference level may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same patient. In yet another embodiment, a reference sample, reference cell, reference tissue, control sample, control cell, control tissue, or reference level is obtained from a healthy and/or non-diseased part of the body (e.g., tissues or cells) of an individual who is not the patient. In even another embodiment, a reference sample, reference cell, reference tissue, control sample, control cell, control tissue, or reference level is obtained from an untreated tissue and/or cell of the body of an individual who is not the patient. In a further embodiment, a reference level may be obtained from a population of individuals (e.g., a population of patients having a disorder such as cancer (e.g., lung cancer (e.g., SCLC, e.g., ES-SCLC or LS-SCLC)), including a population of patients that does not include the patient being assessed or treated according to a method disclosed herein.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

The phrase “based on” when used herein means that the information about one or more biomarkers is used to inform a treatment decision, information provided on a package insert, or marketing/promotional guidance, and the like. For example, a patient may be selected for an anti-cancer therapy and/or treated with an anti-cancer therapy based on classification of the patient as disclosed herein, e.g., by assignment of the patient's tumor sample into one of the following four subtypes based on the transcriptional profile of the patient's tumor: NE-I, NE-N, NE-A, or nNE-I.

As used herein, the terms “mutational load,” “mutation load,” “mutational burden,” “tumor mutational burden score,” “TMB score,” “tissue tumor mutational burden score,” and “tTMB score” each of which may be used interchangeably, refer to the level (e.g., number) of an alteration (e.g., one or more alterations, e.g., one or more somatic alterations) per a pre-selected unit (e.g., per megabase) in a pre-determined set of genes (e.g., in the coding regions of the pre-determined set of genes) detected in a tumor tissue sample (e.g., a formalin-fixed and paraffin-embedded (FFPE) tumor sample, an archival tumor sample, a fresh tumor sample, or a frozen tumor sample). The tTMB score can be measured, for example, on a whole genome or exome basis, or on the basis of a subset of the genome or exome. In certain embodiments, the tTMB score measured on the basis of a subset of the genome or exome can be extrapolated to determine a whole genome or exome mutation load. In some embodiments, a tTMB score refers to the level of accumulated somatic mutations within a patient. The tTMB score may refer to accumulated somatic mutations in a patient with cancer (e.g., lung cancer (e.g., SCLC, e.g., ES-SCLC or LS-SCLC)). In some embodiments, a tTMB score refers to the accumulated mutations in the whole genome of a patient. In some embodiments, a tTMB score refers to the accumulated mutations within a particular tissue sample (e.g., tumor tissue sample biopsy, e.g., a lung tumor sample) collected from a patient. For example, in some embodiments, mutation load may be assessed as described in any one the following publications: U.S. Pat. No. 11,279,767; and U.S. Patent Application Publication Nos. US 2018/0363066, US 2019/0025308, and US 2019/0219586.

As used herein, the terms “blood tumor mutational burden score,” “blood tumor mutation burden score,” and “bTMB score,” each of which may be used interchangeably, refer to a numerical value that reflects the number of somatic mutations detected in a blood sample (e.g., a whole blood sample, a plasma sample, a serum sample, or a combination thereof) obtained from an individual (e.g., an individual at risk of or having a cancer). The bTMB score can be measured, for example, on a whole genome or exome basis (e.g., by whole exome sequencing (WES)), or on the basis of a subset of the genome or exome (e.g., a predetermined set of genes).

The terms “somatic variant,” “somatic mutation,” or “somatic alteration” refer to a genetic alteration occurring in the somatic tissues (e.g., cells outside the germline). Examples of genetic alterations include, but are not limited to, point mutations (e.g., the exchange of a single nucleotide for another (e.g., silent mutations, missense mutations, and nonsense mutations)), insertions and deletions (e.g., the addition and/or removal of one or more nucleotides (e.g., indels)), amplifications, gene duplications, copy number alterations (CNAs), rearrangements, and splice variants. The presence of particular mutations can be associated with disease states (e.g., cancer, e.g., lung cancer (e.g., SCLC, e.g., ES-SCLC or LS-SCLC)).

The term “multiplex-PCR” refers to a single PCR reaction carried out on nucleic acid obtained from a single source (e.g., an individual) using more than one primer set for the purpose of amplifying two or more DNA sequences in a single reaction.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” or “quantitative PCR” or “qPCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

“RNA sequencing” or “RNA-seq,” also called “Whole Transcriptome Shotgun Sequencing (WTSS),” refers to the use of high-throughput sequencing technologies to sequence and/or quantify cDNA to obtain information about a sample's RNA content. Publications describing RNA-seq include: Wang et al. Nature Reviews Genetics 10 (1):57-63, 2009; Ryan et al. BioTechniques 45(1): 81-94, 2008; and Maher et al. Nature 458(7234):97-101, 2009.

As used herein, the term “induction phase” refers to a series of one or more dosing cycles (e.g., about 4 cycles) of one or more therapeutic agents (e.g., a PD-1 axis binding antagonist and/or one or more chemotherapeutic agents (e.g., carboplatin and/or etoposide)) administered to a subject, wherein the one or more dosing cycles are optionally followed by a maintenance phase.

The term “maintenance phase” as used herein refers to a series of one or more dosing cycles of one or more therapeutic agents (e.g., a PD-1 axis binding antagonist and/or one or more chemotherapeutic agents (e.g., carboplatin and/or etoposide)) that are administered to a subject subsequent to an induction phase. In some instances, the maintenance phase is initiated only if the subject did not experience disease progression or unacceptable toxicity during the induction phase. The induction phase and maintenance phase may or may not comprise use of the same therapeutic agents. For example, in some instances, the induction phase includes use of a PD-1 axis binding antagonist and one or more chemotherapeutic agents (e.g., carboplatin and/or etoposide), and the maintenance phase includes use of a PD-1 axis binding antagonist.

### II. Methods of Classifying Lung Cancer

Provided herein are methods for classifying lung cancer (e.g., SCLC, e.g., ES-SCLC or LS-SCLC), including in the 1L treatment setting), which may involve assigning a sample (e.g., a tumor sample) from the patient into a subtype as disclosed herein.

In one example, provided herein is a method of classifying a lung cancer (e.g., SCLC, e.g., ES-SCLC or LS-SCLC), including in the 1L treatment setting) in a human patient, the method comprising assigning a sample from the patient into one of the following four subtypes based on a transcriptional profile of the patient's tumor: neuroendocrine inflamed (NE-I), neuroendocrine NEUROD-driven (NE-N), neuroendocrine achaete-scute homolog 1 (ASCL1)-driven (NE-A), or non-neuroendocrine inflamed (nNE-I), thereby classifying the lung cancer in the patient.

In some examples, the transcriptional profile has been provided by assaying mRNA in the sample from the patient. In some examples, the transcriptional profile has been inferred by assaying orthogonal molecules, e.g., DNA, e.g., cfDNA, protein, glycoprotein, and the like. In some examples, the sample is a tumor tissue sample. In some examples, the sample is a liquid biopsy, a blood sample, e.g., serum or plasma, a sputum sample, a urine sample, or the like. In some examples, the transcriptional profile has been provided by assaying mRNA in a tumor tissue sample from the patient.

Any suitable approach for assaying mRNA may be used. In some examples, assaying mRNA in the tumor sample from the patient comprises RNA sequencing (RNA-seq), reverse transcription-quantitative polymerase chain reaction (RT-qPCR), qPCR, multiplex qPCR or RT-qPCR, microarray analysis, serial analysis of gene expression (SAGE), MASSARRAY® technique, in situ hybridization (ISH), or a combination thereof.

In some particular examples, assaying mRNA in the sample from the patient comprises RNA-seq.

Any suitable approach for assaying the one or more orthogonal molecules, e.g., DNA, e.g., cfDNA, proteins, glycoprotein, can be used. In some examples, the assays examine cfDNA chromatin accessibility, serum proteomics, and/or cfDNA methylation. In some examples, assaying the one or more orthogonal molecules comprises immunohistochemistry (“IHC”), Western blot analysis, immunoprecipitation, molecular binding assays, ELISA, ELIFA, flow cytometry, fluorescence activated cell sorting (“FACS”), MASSARRAY®, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, massively parallel DNA sequencing (e.g., next-generation sequencing), NANOSTRING®, polymerase chain reaction (PCR), including quantitative real time PCR (qRT-PCR) and/or reverse transcription-quantitative polymerase chain reaction (RT-qPCR), and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays known to one of skill in the art that can be performed by protein, gene, and/or tissue array analysis.

In another example, provided herein is a method of classifying a lung cancer (e.g., SCLC, e.g., ES-SCLC or LS-SCLC), including in the 1L treatment setting) in a human patient, the method comprising (a) assaying mRNA in a tumor sample from the patient to provide a transcriptional profile of the patient's tumor; and (b) assigning the patient's tumor sample into one of the following four subtypes based on the transcriptional profile of the patient's tumor: NE-I, NE-N, NE-A, or nNE-I, thereby classifying the SCLC in the patient.

In some examples, step (b) comprises assigning the patient's tumor sample into one of the following four subtypes using a machine learning classifier based on the transcriptional profile of the patient's tumor: NE-I, NE-N, NE-A, or nNE-I.

In some examples, the patient is previously untreated for the lung cancer (e.g., SCLC, e.g., ES-SCLC or LS-SCLC). In some examples, the patient has received a previous treatment for the lung cancer (e.g., SCLC, e.g., ES-SCLC or LS-SCLC). In some examples, the patient is chemotherapy-naïve.

Any suitable approach can be used to identify clusters into which a patient's sample (e.g., tumor sample) may be assigned. For example, in some examples, subtypes are identified by non-negative matrix factorization (NMF; see, e.g., Lee et al. Nature 401 (6755): 788-791, 1999 and Brunet et al. Proc. Nat'l Acad. Sci. USA 101:4164-4169, 2004), hierarchical clustering (see, e.g., Eisen et al. Proc. Nat'l Acad. Sci. USA 95 (25): 14863-8, 1998), partition clustering (e.g., K-means clustering, K-medoids clustering, or partitioning around medoids (PAM, see, e.g., Kaufman et al. Finding Groups in Data: John Wiley and Sons, Inc. 2008, pages 68-125)), model-based clustering (e.g., gaussian mixture models), principal component analysis, clustering with deep learning (see, e.g., Li et al. Nat. Commun. 11:2338, 2020), self-organizing map (see, e.g., Kohonen et al. Biol. Cybernet. 43 (1): 59-69, 1982), density-based spatial clustering of applications with noise (DBSCAN, see, e.g., Ester et al. Proceedings of the Second International Conference on Knowledge Discovery and Data Mining; Portland, Oregon: 3001507: AAAI Press; 1996. p. 226-31), and the like. In some examples, hierarchical clustering may include single-linkage, average-linkage, or complete-linkage hierarchical clustering algorithms. Reviews of exemplary clustering approaches are provided, e.g., in Oyalade et al. Bioinform. And Biol. Insights 10:237-253, 2016; Vidman et al. PLOS One 14 (12) e0219102, 2019; and Jamail and Moussa, IntechOpen (DOI: 10.5772/intechopen.94069). In particular examples, subtypes are identified by non-negative NMF, e.g., as described herein in Example 1.

In some examples, RNA-seq count data may be transformed prior to cluster analysis. Any suitable transformation approach can be used, e.g., logarithmic transformation (e.g., log 2-transformation), variance stabilizing transformation, eight data transformation, and the like.

In some examples, the four subtypes are identified by NMF. In some examples, the four subtypes identified by NMF are based on a set of genes representing the top 10% most variable genes in a population of patients having SCLC (e.g., ES-SCLC or LS-SCLC, including in the 1L treatment setting).

Any of the methods described herein may include classification of a patient's sample into a subtype, e.g., any subtype identified herein. For example, machine learning algorithms can be used to develop a classifier from gene expression data. Any suitable machine learning algorithm can be used, including supervised learning (e.g., decision tree, random forest, gradient boost machine (GBM), CATBOOST, XGBOOST, support vector machine (SVM), PCA, K-nearest neighbor, and naïve Bayes) and unsupervised learning approaches. In particular instances, the machine learning algorithm is a random forest algorithm, as described, e.g., in Example 1. For example, a classifier can be developed using the random forest machine learning algorithm (e.g., using the R package randomForest). The random forest classifier can be learned on a training gene set and then used to predict the cluster (e.g., NMF classes) in a second gene set. In other instances, K-means clustering, K-medoids clustering, or PAM can be used for classification.

In some examples, a classifier may be used to assign a patient's tumor to a subtype as disclosed herein. In some examples, a classifier comprising the set of genes set forth in Table 1, or any subset thereof, is used to assign a patient's tumor to a subtype as disclosed herein. The Gene ID numbers in Table 1 represent Ensembl Gene IDs.

TABLE 1

| Gene ID | Gene ID | Gene ID |
|---|---|---|
| ENSG00000104435 | ENSG00000107317 | ENSG00000159712 |
| ENSG00000198183 | ENSG00000241859 | ENSG00000198121 |
| ENSG00000185559 | ENSG00000230863 | ENSG00000118946 |
| ENSG00000125999 | ENSG00000256151 | ENSG00000149927 |
| ENSG00000155511 | ENSG00000168589 | ENSG00000237940 |
| ENSG00000263639 | ENSG00000223513 | ENSG00000276670 |
| ENSG00000233864 | ENSG00000280079 | ENSG00000273925 |
| ENSG00000231754 | ENSG00000180229 | ENSG00000206503 |
| ENSG00000161055 | ENSG00000226816 | ENSG00000232487 |
| ENSG00000110680 | ENSG00000154274 | ENSG00000257151 |
| ENSG00000124780 | ENSG00000011201 | ENSG00000256293 |
| ENSG00000131002 | ENSG00000284624 | ENSG00000181634 |
| ENSG00000186960 | ENSG00000242686 | ENSG00000261628 |
| ENSG00000198692 | ENSG00000199990 | ENSG00000187017 |
| ENSG00000249395 | ENSG00000232679 | ENSG00000248359 |
| ENSG00000165379 | ENSG00000270945 | ENSG00000150681 |
| ENSG00000102924 | ENSG00000011347 | ENSG00000100302 |
| ENSG00000272304 | ENSG00000255191 | ENSG00000230922 |
| ENSG00000141668 | ENSG00000127152 | ENSG00000162639 |
| ENSG00000146166 | ENSG00000122188 | ENSG00000074410 |
| ENSG00000057149 | ENSG00000064692 | ENSG00000102349 |
| ENSG00000136531 | ENSG00000112619 | ENSG00000104814 |
| ENSG00000255794 | ENSG00000105877 | ENSG00000120327 |
| ENSG00000166670 | ENSG00000091664 | ENSG00000238835 |
| ENSG00000162992 | ENSG00000254630 | ENSG00000253978 |
| ENSG00000226555 | ENSG00000161509 | ENSG00000254610 |
| ENSG00000280989 | ENSG00000185272 | ENSG00000183762 |
| ENSG00000170091 | ENSG00000280207 | ENSG00000146005 |
| ENSG00000214548 | ENSG00000157214 | ENSG00000146674 |
| ENSG00000111262 | ENSG00000169855 | ENSG00000065717 |
| ENSG00000249413 | ENSG00000232015 | ENSG00000247134 |
| ENSG00000165246 | ENSG00000260314 | ENSG00000170006 |
| ENSG00000016082 | ENSG00000145920 | ENSG00000010030 |
| ENSG00000129824 | ENSG00000245857 | ENSG00000275327 |
| ENSG00000251536 | ENSG00000164761 | ENSG00000253173 |
| ENSG00000105251 | ENSG00000136542 | ENSG00000134198 |
| ENSG00000166959 | ENSG00000255207 | ENSG00000153898 |
| ENSG00000258536 | ENSG00000171533 | ENSG00000206680 |
| ENSG00000125820 | ENSG00000175318 | ENSG00000159189 |
| ENSG00000211935 | ENSG00000186710 | ENSG00000124613 |
| ENSG00000225746 | ENSG00000183742 | ENSG00000062038 |
| ENSG00000114374 | ENSG00000275591 | ENSG00000104059 |
| ENSG00000066032 | ENSG00000279586 | ENSG00000243297 |
| ENSG00000107105 | ENSG00000076344 | ENSG00000262119 |
| ENSG00000148346 | ENSG00000189127 | ENSG00000278816 |
| ENSG00000018236 | ENSG00000253293 | ENSG00000264937 |
| ENSG00000211638 | ENSG00000284700 | ENSG00000218730 |
| ENSG00000256463 | ENSG00000131781 | ENSG00000253395 |
| ENSG00000260597 | ENSG00000280099 | ENSG00000278017 |
| ENSG00000105388 | ENSG00000173068 | ENSG00000165458 |
| ENSG00000149021 | ENSG00000237541 | ENSG00000200047 |
| ENSG00000280196 | ENSG00000179314 | ENSG00000234685 |
| ENSG00000170689 | ENSG00000258915 | ENSG00000237478 |
| ENSG00000226702 | ENSG00000162599 | ENSG00000108379 |
| ENSG00000181201 | ENSG00000172318 | ENSG00000179915 |
| ENSG00000260197 | ENSG00000250387 | ENSG00000171522 |
| ENSG00000272763 | ENSG00000259447 | ENSG00000187391 |
| ENSG00000171246 | ENSG00000135480 | ENSG00000121807 |
| ENSG00000184613 | ENSG00000137571 | ENSG00000162576 |
| ENSG00000257680 | ENSG00000260224 | ENSG00000213060 |
| ENSG00000151952 | ENSG00000177103 | ENSG00000231217 |
| ENSG00000186474 | ENSG00000167034 | ENSG00000223583 |
| ENSG00000080644 | ENSG00000214676 | ENSG00000123104 |
| ENSG00000268119 | ENSG00000121039 | ENSG00000276633 |
| ENSG00000204398 | ENSG00000147255 | ENSG00000232081 |
| ENSG00000128422 | ENSG00000185681 | ENSG00000186297 |
| ENSG00000279930 | ENSG00000239975 | ENSG00000213950 |
| ENSG00000117983 | ENSG00000251050 | ENSG00000180318 |
| ENSG00000284395 | ENSG00000163520 | ENSG00000279074 |
| ENSG00000211663 | ENSG00000166863 | ENSG00000171051 |
| ENSG00000245750 | ENSG00000100078 | ENSG00000213305 |
| ENSG00000120068 | ENSG00000231376 | ENSG00000236542 |
| ENSG00000263146 | ENSG00000172575 | ENSG00000241644 |
| ENSG00000265179 | ENSG00000166960 | ENSG00000141431 |
| ENSG00000180806 | ENSG00000248282 | ENSG00000276653 |
| ENSG00000079689 | ENSG00000213549 | ENSG00000143382 |
| ENSG00000162896 | ENSG00000133321 | ENSG00000256937 |
| ENSG00000118702 | ENSG00000108370 | ENSG00000175879 |
| ENSG00000251381 | ENSG00000254535 | ENSG00000162399 |
| ENSG00000282142 | ENSG00000182195 | ENSG00000241770 |
| ENSG00000254302 | ENSG00000260859 | ENSG00000279410 |
| ENSG00000257989 | ENSG00000207406 | ENSG00000274020 |
| ENSG00000196090 | ENSG00000088002 | ENSG00000265254 |
| ENSG00000211934 | ENSG00000124212 | ENSG00000075213 |
| ENSG00000170373 | ENSG00000126709 | ENSG00000220884 |
| ENSG00000236028 | ENSG00000245025 | ENSG00000138271 |
| ENSG00000154620 | ENSG00000139874 | ENSG00000164695 |
| ENSG00000210112 | ENSG00000179761 | ENSG00000229057 |
| ENSG00000211668 | ENSG00000126950 | ENSG00000138435 |
| ENSG00000227189 | ENSG00000271394 | ENSG00000157111 |
| ENSG00000172137 | ENSG00000026751 | ENSG00000120549 |
| ENSG00000211598 | ENSG00000154316 | ENSG00000187601 |
| ENSG00000211941 | ENSG00000153823 | ENSG00000224388 |
| ENSG00000253853 | ENSG00000092969 | ENSG00000265107 |
| ENSG00000227722 | ENSG00000237751 | ENSG00000117507 |
| ENSG00000106236 | ENSG00000076716 | ENSG00000196839 |
| ENSG00000211965 | ENSG00000267909 | ENSG00000189420 |
| ENSG00000171951 | ENSG00000244292 | ENSG00000124813 |
| ENSG00000250366 | ENSG00000223117 | ENSG00000164841 |
| ENSG00000259485 | ENSG00000224848 | ENSG00000258591 |
| ENSG00000226572 | ENSG00000211650 | ENSG00000242551 |
| ENSG00000142319 | ENSG00000108947 | ENSG00000123892 |
| ENSG00000211673 | ENSG00000248803 | ENSG00000204683 |
| ENSG00000253137 | ENSG00000196411 | ENSG00000263727 |
| ENSG00000211959 | ENSG00000099953 | ENSG00000234816 |
| ENSG00000255209 | ENSG00000211751 | ENSG00000072694 |
| ENSG00000186081 | ENSG00000225292 | ENSG00000147642 |
| ENSG00000108511 | ENSG00000199856 | ENSG00000124839 |
| ENSG00000189056 | ENSG00000087494 | ENSG00000167779 |
| ENSG00000067048 | ENSG00000109758 | ENSG00000175287 |
| ENSG00000211966 | ENSG00000267746 | ENSG00000140368 |
| ENSG00000258636 | ENSG00000276508 | ENSG00000183715 |
| ENSG00000267793 | ENSG00000253250 | ENSG00000149534 |
| ENSG00000184564 | ENSG00000267766 | ENSG00000151967 |
| ENSG00000207375 | ENSG00000204962 | ENSG00000120915 |
| ENSG00000226903 | ENSG00000115414 | ENSG00000176845 |
| ENSG00000099725 | ENSG00000254349 | ENSG00000242853 |
| ENSG00000233204 | ENSG00000225489 | ENSG00000067840 |
| ENSG00000187323 | ENSG00000265579 | ENSG00000170323 |
| ENSG00000186487 | ENSG00000252960 | ENSG00000139832 |
| ENSG00000130558 | ENSG00000048462 | ENSG00000243544 |
| ENSG00000215182 | ENSG00000171017 | ENSG00000170558 |
| ENSG00000179455 | ENSG00000280255 | ENSG00000181690 |
| ENSG00000211662 | ENSG00000121898 | ENSG00000117091 |
| ENSG00000182968 | ENSG00000163235 | ENSG00000262001 |
| ENSG00000211964 | ENSG00000165309 | ENSG00000166669 |
| ENSG00000036565 | ENSG00000258794 | ENSG00000125430 |
| ENSG00000211666 | ENSG00000270988 | ENSG00000104611 |
| ENSG00000205420 | ENSG00000102043 | ENSG00000086619 |
| ENSG00000241451 | ENSG00000188386 | ENSG00000179284 |
| ENSG00000251652 | ENSG00000282826 | ENSG00000109193 |
| ENSG00000148357 | ENSG00000253369 | ENSG00000149633 |
| ENSG00000048540 | ENSG00000251656 | ENSG00000132256 |
| ENSG00000178363 | ENSG00000170801 | ENSG00000119699 |
| ENSG00000211951 | ENSG00000237517 | ENSG00000106003 |
| ENSG00000188039 | ENSG00000237133 | ENSG00000137819 |
| ENSG00000256085 | ENSG00000166265 | ENSG00000187498 |
| ENSG00000211896 | ENSG00000167772 | ENSG00000002586 |
| ENSG00000184672 | ENSG00000242358 | ENSG00000157557 |
| ENSG00000211950 | ENSG00000130203 | ENSG00000279691 |
| ENSG00000124107 | ENSG00000227082 | ENSG00000255177 |
| ENSG00000212158 | ENSG00000278926 | ENSG00000242534 |
| ENSG00000244575 | ENSG00000235761 | ENSG00000183918 |
| ENSG00000181143 | ENSG00000171759 | ENSG00000244044 |
| ENSG00000275996 | ENSG00000255248 | ENSG00000164120 |
| ENSG00000284513 | ENSG00000130303 | ENSG00000135063 |
| ENSG00000276775 | ENSG00000201967 | ENSG00000201428 |
| ENSG00000115266 | ENSG00000226431 | ENSG00000231485 |
| ENSG00000211949 | ENSG00000105374 | ENSG00000116741 |
| ENSG00000165731 | ENSG00000258033 | ENSG00000078900 |
| ENSG00000197757 | ENSG00000284727 | ENSG00000261051 |
| ENSG00000087510 | ENSG00000184012 | ENSG00000135472 |
| ENSG00000211677 | ENSG00000228010 | ENSG00000162804 |
| ENSG00000105048 | ENSG00000175832 | ENSG00000273300 |
| ENSG00000211945 | ENSG00000278732 | ENSG00000224034 |
| ENSG00000211962 | ENSG00000176532 | ENSG00000278794 |
| ENSG00000211937 | ENSG00000230838 | ENSG00000129538 |
| ENSG00000124237 | ENSG00000204961 | ENSG00000178695 |
| ENSG00000012817 | ENSG00000272053 | ENSG00000239726 |
| ENSG00000183878 | ENSG00000148926 | ENSG00000109255 |
| ENSG00000278196 | ENSG00000259519 | ENSG00000122877 |
| ENSG00000095981 | ENSG00000134028 | ENSG00000271138 |
| ENSG00000240393 | ENSG00000106819 | ENSG00000262158 |
| ENSG00000198046 | ENSG00000260910 | ENSG00000223969 |
| ENSG00000164458 | ENSG00000222724 | ENSG00000228421 |
| ENSG00000124429 | ENSG00000269902 | ENSG00000267361 |
| ENSG00000223941 | ENSG00000211694 | ENSG00000034053 |
| ENSG00000273203 | ENSG00000104447 | ENSG00000218766 |
| ENSG00000167757 | ENSG00000118495 | ENSG00000102854 |
| ENSG00000229686 | ENSG00000166924 | ENSG00000156535 |
| ENSG00000259306 | ENSG00000274578 | ENSG00000230006 |
| ENSG00000270550 | ENSG00000115041 | ENSG00000122176 |
| ENSG00000211679 | ENSG00000119147 | ENSG00000164438 |
| ENSG00000211653 | ENSG00000244349 | ENSG00000277954 |
| ENSG00000199879 | ENSG00000235244 | ENSG00000183929 |
| ENSG00000241351 | ENSG00000238142 | ENSG00000169469 |
| ENSG00000237438 | ENSG00000127863 | ENSG00000163599 |
| ENSG00000123307 | ENSG00000269680 | ENSG00000201448 |
| ENSG00000189223 | ENSG00000161647 | ENSG00000279269 |
| ENSG00000164651 | ENSG00000167880 | ENSG00000134827 |
| ENSG00000273179 | ENSG00000162444 | ENSG00000161860 |
| ENSG00000214288 | ENSG00000274173 | ENSG00000056487 |
| ENSG00000166426 | ENSG00000162493 | ENSG00000214796 |
| ENSG00000179813 | ENSG00000234112 | ENSG00000142235 |
| ENSG00000141433 | ENSG00000026508 | ENSG00000118508 |
| ENSG00000243466 | ENSG00000164626 | ENSG00000278469 |
| ENSG00000182747 | ENSG00000213937 | ENSG00000223749 |
| ENSG00000211669 | ENSG00000199218 | ENSG00000179219 |
| ENSG00000211947 | ENSG00000022267 | ENSG00000265763 |
| ENSG00000272034 | ENSG00000171811 | ENSG00000254897 |
| ENSG00000211660 | ENSG00000100918 | ENSG00000284709 |
| ENSG00000280222 | ENSG00000269916 | ENSG00000140287 |
| ENSG00000107518 | ENSG00000197594 | ENSG00000276384 |
| ENSG00000147231 | ENSG00000105219 | ENSG00000162595 |
| ENSG00000272619 | ENSG00000244649 | ENSG00000122691 |
| ENSG00000211659 | ENSG00000198576 | ENSG00000179583 |
| ENSG00000211938 | ENSG00000180440 | ENSG00000228784 |
| ENSG00000278041 | ENSG00000152582 | ENSG00000247516 |
| ENSG00000183654 | ENSG00000263426 | ENSG00000272541 |
| ENSG00000067842 | ENSG00000137960 | ENSG00000163576 |
| ENSG00000244734 | ENSG00000203808 | ENSG00000050344 |
| ENSG00000188452 | ENSG00000196344 | ENSG00000267102 |
| ENSG00000252316 | ENSG00000160233 | ENSG00000133216 |
| ENSG00000101144 | ENSG00000248283 | ENSG00000146001 |
| ENSG00000211640 | ENSG00000228551 | ENSG00000239247 |
| ENSG00000085741 | ENSG00000214578 | ENSG00000278462 |
| ENSG00000156150 | ENSG00000273618 | ENSG00000269524 |
| ENSG00000251521 | ENSG00000276417 | ENSG00000111664 |
| ENSG00000256923 | ENSG00000143416 | ENSG00000269821 |
| ENSG00000203414 | ENSG00000150672 | ENSG00000231806 |
| ENSG00000120251 | ENSG00000118733 | ENSG00000267550 |
| ENSG00000082556 | ENSG00000207201 | ENSG00000138400 |
| ENSG00000229880 | ENSG00000145934 | ENSG00000241255 |
| ENSG00000267287 | ENSG00000237720 | ENSG00000266875 |
| ENSG00000164220 | ENSG00000062524 | ENSG00000235663 |
| ENSG00000260947 | ENSG00000233820 | ENSG00000163191 |
| ENSG00000256995 | ENSG00000231738 | ENSG00000047365 |
| ENSG00000117154 | ENSG00000105737 | ENSG00000153993 |
| ENSG00000161082 | ENSG00000233730 | ENSG00000125895 |
| ENSG00000264015 | ENSG00000130518 | ENSG00000238825 |
| ENSG00000237321 | ENSG00000261286 | ENSG00000177335 |
| ENSG00000211897 | ENSG00000240677 | ENSG00000171791 |
| ENSG00000105880 | ENSG00000276547 | ENSG00000153956 |
| ENSG00000129167 | ENSG00000174469 | ENSG00000151388 |
| ENSG00000188536 | ENSG00000165300 | ENSG00000082074 |
| ENSG00000077279 | ENSG00000240065 | ENSG00000175600 |
| ENSG00000066735 | ENSG00000155792 | ENSG00000234614 |
| ENSG00000211893 | ENSG00000253731 | ENSG00000182492 |
| ENSG00000273703 | ENSG00000255163 | ENSG00000112303 |
| ENSG00000033122 | ENSG00000168126 | ENSG00000274447 |
| ENSG00000197153 | ENSG00000196557 | ENSG00000266651 |
| ENSG00000146352 | ENSG00000174460 | ENSG00000277998 |
| ENSG00000211592 | ENSG00000204335 | ENSG00000142632 |
| ENSG00000259663 | ENSG00000253485 | ENSG00000214280 |
| ENSG00000272787 | ENSG00000219928 | ENSG00000225217 |
| ENSG00000153253 | ENSG00000179873 | ENSG00000246095 |
| ENSG00000280650 | ENSG00000250020 | ENSG00000165188 |
| ENSG00000239268 | ENSG00000088280 | ENSG00000106078 |
| ENSG00000214336 | ENSG00000198203 | ENSG00000256064 |
| ENSG00000162706 | ENSG00000198125 | ENSG00000184357 |
| ENSG00000231165 | ENSG00000233273 | ENSG00000146530 |
| ENSG00000101210 | ENSG00000105479 | ENSG00000157570 |
| ENSG00000159784 | ENSG00000271509 | ENSG00000267756 |
| ENSG00000148935 | ENSG00000279662 | ENSG00000275160 |
| ENSG00000239951 | ENSG00000275302 | ENSG00000199363 |
| ENSG00000124939 | ENSG00000186868 | ENSG00000005102 |
| ENSG00000237515 | ENSG00000163694 | ENSG00000271081 |
| ENSG00000211892 | ENSG00000206948 | ENSG00000223665 |
| ENSG00000276788 | ENSG00000141314 | ENSG00000138131 |
| ENSG00000132429 | ENSG00000180340 | ENSG00000253414 |
| ENSG00000275063 | ENSG00000227835 | ENSG00000124191 |
| ENSG00000211933 | ENSG00000136883 | ENSG00000278654 |
| ENSG00000078399 | ENSG00000182326 | ENSG00000138792 |
| ENSG00000236212 | ENSG00000117228 | ENSG00000273274 |
| ENSG00000139219 | ENSG00000153157 | ENSG00000143473 |
| ENSG00000184156 | ENSG00000206828 | ENSG00000116132 |
| ENSG00000198216 | ENSG00000197233 | ENSG00000231490 |
| ENSG00000065609 | ENSG00000204219 | ENSG00000165495 |
| ENSG00000199377 | ENSG00000163017 | ENSG00000234405 |
| ENSG00000169562 | ENSG00000135519 | ENSG00000172771 |
| ENSG00000198838 | ENSG00000250977 | ENSG00000174807 |
| ENSG00000196611 | ENSG00000162490 | ENSG00000225345 |
| ENSG00000157542 | ENSG00000135750 | ENSG00000175352 |
| ENSG00000166828 | ENSG00000158270 | ENSG00000092850 |
| ENSG00000106633 | ENSG00000162981 | ENSG00000279554 |
| ENSG00000106541 | ENSG00000145198 | ENSG00000250742 |
| ENSG00000279358 | ENSG00000234745 | ENSG00000266601 |
| ENSG00000121904 | ENSG00000136014 | ENSG00000257094 |
| ENSG00000112852 | ENSG00000260549 | ENSG00000131016 |
| ENSG00000244437 | ENSG00000215881 | ENSG00000187672 |
| ENSG00000143184 | ENSG00000165966 | ENSG00000126460 |
| ENSG00000184408 | ENSG00000174343 | ENSG00000143036 |
| ENSG00000136750 | ENSG00000106366 | ENSG00000231878 |
| ENSG00000223403 | ENSG00000237943 | ENSG00000184933 |
| ENSG00000159212 | ENSG00000225783 | ENSG00000268659 |
| ENSG00000279725 | ENSG00000211970 | ENSG00000111816 |
| ENSG00000175894 | ENSG00000141384 | ENSG00000008517 |
| ENSG00000276644 | ENSG00000134339 | ENSG00000264350 |
| ENSG00000067646 | ENSG00000108551 | ENSG00000272810 |
| ENSG00000235726 | ENSG00000172061 | ENSG00000243538 |
| ENSG00000091656 | ENSG00000241288 | ENSG00000232891 |
| ENSG00000254709 | ENSG00000147138 | ENSG00000077713 |
| ENSG00000170561 | ENSG00000251237 | ENSG00000271002 |
| ENSG00000138755 | ENSG00000117600 | ENSG00000215187 |
| ENSG00000078328 | ENSG00000232415 | ENSG00000091972 |
| ENSG00000211955 | ENSG00000181409 | ENSG00000121900 |
| ENSG00000273442 | ENSG00000112562 | ENSG00000185862 |
| ENSG00000211943 | ENSG00000163359 | ENSG00000008311 |
| ENSG00000150594 | ENSG00000246695 | ENSG00000172817 |
| ENSG00000259957 | ENSG00000273367 | ENSG00000182256 |
| ENSG00000119042 | ENSG00000232229 | ENSG00000173890 |
| ENSG00000144481 | ENSG00000165617 | ENSG00000206341 |
| ENSG00000170579 | ENSG00000180785 | ENSG00000271798 |
| ENSG00000262943 | ENSG00000241942 | ENSG00000279995 |
| ENSG00000179869 | ENSG00000243516 | ENSG00000272942 |
| ENSG00000197261 | ENSG00000201403 | ENSG00000233613 |
| ENSG00000074211 | ENSG00000132386 | ENSG00000137473 |
| ENSG00000141449 | ENSG00000134258 | ENSG00000250081 |
| ENSG00000183091 | ENSG00000221887 | ENSG00000012232 |
| ENSG00000200972 | ENSG00000242779 | ENSG00000171236 |
| ENSG00000253857 | ENSG00000214189 | ENSG00000280212 |
| ENSG00000235833 | ENSG00000169116 | ENSG00000152661 |
| ENSG00000268081 | ENSG00000172201 | ENSG00000125355 |
| ENSG00000253642 | ENSG00000236153 | ENSG00000246214 |
| ENSG00000260614 | ENSG00000268545 | ENSG00000142149 |
| ENSG00000180616 | ENSG00000198643 | ENSG00000126016 |
| ENSG00000183117 | ENSG00000124507 | ENSG00000173281 |
| ENSG00000140015 | ENSG00000261804 | ENSG00000227486 |
| ENSG00000006747 | ENSG00000149926 | ENSG00000002726 |
| ENSG00000164418 | ENSG00000282964 | ENSG00000279277 |
| ENSG00000188763 | ENSG00000111218 | ENSG00000259235 |
| ENSG00000236562 | ENSG00000204291 | ENSG00000134871 |
| ENSG00000268104 | ENSG00000167741 | ENSG00000227973 |
| ENSG00000125285 | ENSG00000149050 | ENSG00000280375 |
| ENSG00000160862 | ENSG00000248231 | ENSG00000123094 |
| ENSG00000218274 | ENSG00000145506 | ENSG00000131979 |
| ENSG00000204967 | ENSG00000242220 | ENSG00000167183 |
| ENSG00000165553 | ENSG00000137573 | ENSG00000176771 |
| ENSG00000270460 | ENSG00000267554 | ENSG00000244124 |
| ENSG00000187045 | ENSG00000280027 | ENSG00000197943 |
| ENSG00000211976 | ENSG00000258783 | ENSG00000141854 |
| ENSG00000116147 | ENSG00000091986 | ENSG00000241709 |
| ENSG00000225472 | ENSG00000109339 | ENSG00000155265 |
| ENSG00000224373 | ENSG00000250001 | ENSG00000262902 |
| ENSG00000027644 | ENSG00000116824 | ENSG00000166268 |
| ENSG00000243355 | ENSG00000079482 | ENSG00000226352 |
| ENSG00000241213 | ENSG00000261451 | ENSG00000182508 |
| ENSG00000184292 | ENSG00000187266 | ENSG00000111981 |
| ENSG00000258739 | ENSG00000278231 | ENSG00000118707 |
| ENSG00000240864 | ENSG00000175548 | ENSG00000278272 |
| ENSG00000134443 | ENSG00000254615 | ENSG00000165168 |
| ENSG00000244116 | ENSG00000239911 | ENSG00000118308 |
| ENSG00000137968 | ENSG00000152766 | ENSG00000177106 |
| ENSG00000155052 | ENSG00000185818 | ENSG00000131951 |
| ENSG00000211898 | ENSG00000168993 | ENSG00000183049 |
| ENSG00000214039 | ENSG00000201558 | ENSG00000239701 |
| ENSG00000181790 | ENSG00000167286 | ENSG00000202347 |
| ENSG00000140557 | ENSG00000039139 | ENSG00000249363 |
| ENSG00000149571 | ENSG00000235631 | ENSG00000239570 |
| ENSG00000254938 | ENSG00000254024 | ENSG00000133019 |
| ENSG00000276180 | ENSG00000177575 | ENSG00000142156 |
| ENSG00000280411 | ENSG00000183186 | ENSG00000273891 |
| ENSG00000211956 | ENSG00000114115 | ENSG00000170681 |
| ENSG00000207241 | ENSG00000101230 | ENSG00000105639 |
| ENSG00000130032 | ENSG00000181374 | ENSG00000112309 |
| ENSG00000122756 | ENSG00000132561 | ENSG00000236330 |
| ENSG00000228835 | ENSG00000169715 | ENSG00000279289 |
| ENSG00000256321 | ENSG00000274198 | ENSG00000272512 |
| ENSG00000135373 | ENSG00000224090 | ENSG00000265962 |
| ENSG00000251095 | ENSG00000234409 | ENSG00000147573 |
| ENSG00000224955 | ENSG00000224348 | ENSG00000223865 |
| ENSG00000256654 | ENSG00000173391 | ENSG00000234772 |
| ENSG00000143546 | ENSG00000087495 | ENSG00000261373 |
| ENSG00000213468 | ENSG00000091844 | ENSG00000005243 |
| ENSG00000104327 | ENSG00000247746 | ENSG00000273890 |
| ENSG00000105852 | ENSG00000198719 | ENSG00000131773 |
| ENSG00000241294 | ENSG00000101017 | ENSG00000233593 |
| ENSG00000261116 | ENSG00000171617 | ENSG00000254670 |
| ENSG00000163736 | ENSG00000254537 | ENSG00000171444 |
| ENSG00000148735 | ENSG00000207274 | ENSG00000159409 |
| ENSG00000258861 | ENSG00000258850 | ENSG00000274995 |
| ENSG00000211670 | ENSG00000177822 | ENSG00000283265 |
| ENSG00000082482 | ENSG00000129295 | ENSG00000152760 |
| ENSG00000176165 | ENSG00000225972 | ENSG00000141441 |
| ENSG00000138653 | ENSG00000203797 | ENSG00000095585 |
| ENSG00000231475 | ENSG00000164308 | ENSG00000278058 |
| ENSG00000278975 | ENSG00000181085 | ENSG00000183317 |
| ENSG00000178031 | ENSG00000159399 | ENSG00000114315 |
| ENSG00000183775 | ENSG00000206799 | ENSG00000248527 |
| ENSG00000088538 | ENSG00000171476 | ENSG00000164855 |
| ENSG00000196104 | ENSG00000237082 | ENSG00000164171 |
| ENSG00000201104 | ENSG00000157766 | ENSG00000138316 |
| ENSG00000180264 | ENSG00000258375 | ENSG00000159713 |
| ENSG00000168143 | ENSG00000255571 | ENSG00000264549 |
| ENSG00000174482 | ENSG00000250120 | ENSG00000242020 |
| ENSG00000221539 | ENSG00000258752 | ENSG00000280167 |
| ENSG00000282651 | ENSG00000153820 | ENSG00000250327 |
| ENSG00000172554 | ENSG00000211829 | ENSG00000141294 |
| ENSG00000043355 | ENSG00000132821 | ENSG00000128284 |
| ENSG00000277034 | ENSG00000264113 | ENSG00000279146 |
| ENSG00000224650 | ENSG00000171124 | ENSG00000237186 |
| ENSG00000213988 | ENSG00000254245 | ENSG00000259205 |
| ENSG00000075461 | ENSG00000273107 | ENSG00000133519 |
| ENSG00000223995 | ENSG00000219438 | ENSG00000225671 |
| ENSG00000228089 | ENSG00000156298 | ENSG00000090975 |
| ENSG00000132465 | ENSG00000259895 | ENSG00000090097 |
| ENSG00000211648 | ENSG00000147852 | ENSG00000276842 |
| ENSG00000137077 | ENSG00000154917 | ENSG00000206190 |
| ENSG00000075429 | ENSG00000224407 | ENSG00000203435 |
| ENSG00000175084 | ENSG00000187185 | ENSG00000264169 |
| ENSG00000172005 | ENSG00000253537 | ENSG00000255310 |
| ENSG00000104313 | ENSG00000275395 | ENSG00000261678 |
| ENSG00000187715 | ENSG00000205002 | ENSG00000253958 |
| ENSG00000253047 | ENSG00000169213 | ENSG00000163975 |
| ENSG00000013297 | ENSG00000118997 | ENSG00000090013 |
| ENSG00000086548 | ENSG00000234183 | ENSG00000128165 |
| ENSG00000100314 | ENSG00000229344 | ENSG00000177551 |
| ENSG00000105894 | ENSG00000008196 | ENSG00000279312 |
| ENSG00000166770 | ENSG00000197753 | ENSG00000131097 |
| ENSG00000176956 | ENSG00000122641 | ENSG00000242097 |
| ENSG00000277639 | ENSG00000180720 | ENSG00000164125 |
| ENSG00000099974 | ENSG00000235077 | ENSG00000224669 |
| ENSG00000176728 | ENSG00000116574 | ENSG00000271811 |
| ENSG00000099864 | ENSG00000135835 | ENSG00000198547 |
| ENSG00000124749 | ENSG00000254533 | ENSG00000196503 |
| ENSG00000231764 | ENSG00000265206 | ENSG00000136352 |
| ENSG00000250159 | ENSG00000279741 | ENSG00000053524 |
| ENSG00000178403 | ENSG00000182329 | ENSG00000112137 |
| ENSG00000155754 | ENSG00000269981 | ENSG00000182580 |
| ENSG00000258399 | ENSG00000185736 | ENSG00000275927 |
| ENSG00000203985 | ENSG00000230251 | ENSG00000279770 |
| ENSG00000169245 | ENSG00000063180 | ENSG00000149591 |
| ENSG00000257126 | ENSG00000277749 | ENSG00000064225 |
| ENSG00000125798 | ENSG00000166292 | ENSG00000128040 |
| ENSG00000117069 | ENSG00000173110 | ENSG00000133069 |
| ENSG00000211649 | ENSG00000171595 | ENSG00000137261 |
| ENSG00000007402 | ENSG00000004799 | ENSG00000223573 |
| ENSG00000151572 | ENSG00000152969 | ENSG00000110169 |
| ENSG00000165566 | ENSG00000248787 | ENSG00000197334 |
| ENSG00000116990 | ENSG00000237061 | ENSG00000277245 |
| ENSG00000142973 | ENSG00000278962 | ENSG00000275005 |
| ENSG00000231106 | ENSG00000232104 | ENSG00000154589 |
| ENSG00000254560 | ENSG00000237418 | ENSG00000125869 |
| ENSG00000276048 | ENSG00000111432 | ENSG00000143248 |
| ENSG00000122859 | ENSG00000275896 | ENSG00000255690 |
| ENSG00000188580 | ENSG00000135333 | ENSG00000204334 |
| ENSG00000211890 | ENSG00000254151 | ENSG00000123612 |
| ENSG00000206897 | ENSG00000183287 | ENSG00000197381 |
| ENSG00000232216 | ENSG00000124406 | ENSG00000198780 |
| ENSG00000258419 | ENSG00000182795 | ENSG00000154654 |
| ENSG00000134755 | ENSG00000223963 | ENSG00000268218 |
| ENSG00000198795 | ENSG00000105810 | ENSG00000232714 |
| ENSG00000225689 | ENSG00000270168 | ENSG00000283141 |
| ENSG00000178222 | ENSG00000235888 | ENSG00000273096 |
| ENSG00000260172 | ENSG00000113088 | ENSG00000157103 |
| ENSG00000268089 | ENSG00000151838 | ENSG00000279726 |
| ENSG00000253974 | ENSG00000213186 | ENSG00000172578 |
| ENSG00000211637 | ENSG00000196092 | ENSG00000230521 |
| ENSG00000165194 | ENSG00000258092 | ENSG00000102359 |
| ENSG00000177984 | ENSG00000152527 | ENSG00000105290 |
| ENSG00000124875 | ENSG00000279310 | ENSG00000157404 |
| ENSG00000178150 | ENSG00000121858 | ENSG00000267776 |
| ENSG00000054179 | ENSG00000107562 | ENSG00000043039 |
| ENSG00000007174 | ENSG00000120093 | ENSG00000275666 |
| ENSG00000160097 | ENSG00000177675 | ENSG00000223849 |
| ENSG00000143001 | ENSG00000151025 | ENSG00000261996 |
| ENSG00000135549 | ENSG00000027869 | ENSG00000268896 |
| ENSG00000123454 | ENSG00000138829 | ENSG00000201512 |
| ENSG00000240086 | ENSG00000277632 | ENSG00000272733 |
| ENSG00000188153 | ENSG00000165816 | ENSG00000241081 |
| ENSG00000277269 | ENSG00000069482 | ENSG00000257613 |
| ENSG00000139200 | ENSG00000237621 | ENSG00000104140 |
| ENSG00000231205 | ENSG00000184254 | ENSG00000260196 |
| ENSG00000187416 | ENSG00000252213 | ENSG00000050628 |
| ENSG00000184985 | ENSG00000213244 | ENSG00000246100 |
| ENSG00000110944 | ENSG00000103888 | ENSG00000237807 |
| ENSG00000103449 | ENSG00000133937 | ENSG00000113532 |
| ENSG00000148702 | ENSG00000259513 | ENSG00000145040 |
| ENSG00000277986 | ENSG00000095777 | ENSG00000188523 |
| ENSG00000272108 | ENSG00000272159 | ENSG00000118640 |
| ENSG00000077063 | ENSG00000127954 | ENSG00000253057 |
| ENSG00000184956 | ENSG00000180155 | ENSG00000197747 |
| ENSG00000008394 | ENSG00000225330 | ENSG00000241064 |
| ENSG00000209482 | ENSG00000259345 | ENSG00000230943 |
| ENSG00000200731 | ENSG00000254708 | ENSG00000153563 |
| ENSG00000131746 | ENSG00000138061 | ENSG00000170523 |
| ENSG00000179242 | ENSG00000207168 | ENSG00000259920 |
| ENSG00000266513 | ENSG00000184731 | ENSG00000251152 |
| ENSG00000180730 | ENSG00000147027 | ENSG00000186765 |
| ENSG00000166415 | ENSG00000166562 | ENSG00000234705 |
| ENSG00000154864 | ENSG00000143590 | ENSG00000124664 |
| ENSG00000092421 | ENSG00000159403 | ENSG00000273069 |
| ENSG00000176399 | ENSG00000166407 | ENSG00000168779 |
| ENSG00000092758 | ENSG00000168453 | ENSG00000188906 |
| ENSG00000132872 | ENSG00000204624 | ENSG00000156869 |
| ENSG00000204347 | ENSG00000115165 | ENSG00000144668 |
| ENSG00000171671 | ENSG00000255085 | ENSG00000230148 |
| ENSG00000228206 | ENSG00000103154 | ENSG00000239559 |
| ENSG00000171956 | ENSG00000152192 | ENSG00000273198 |
| ENSG00000211651 | ENSG00000152669 | ENSG00000264057 |
| ENSG00000107159 | ENSG00000143217 | ENSG00000128510 |
| ENSG00000250972 | ENSG00000178538 | ENSG00000106004 |
| ENSG00000279099 | ENSG00000186051 | ENSG00000256540 |
| ENSG00000182050 | ENSG00000250645 | ENSG00000223012 |
| ENSG00000157765 | ENSG00000271958 | ENSG00000241678 |
| ENSG00000183036 | ENSG00000117115 | ENSG00000127418 |
| ENSG00000221439 | ENSG00000141837 | ENSG00000011332 |
| ENSG00000224243 | ENSG00000186952 | ENSG00000172236 |
| ENSG00000221864 | ENSG00000140945 | ENSG00000198626 |
| ENSG00000185518 | ENSG00000171368 | ENSG00000076864 |
| ENSG00000006611 | ENSG00000284678 | ENSG00000223658 |
| ENSG00000126500 | ENSG00000237303 | ENSG00000106113 |
| ENSG00000134569 | ENSG00000277531 | ENSG00000155093 |
| ENSG00000274618 | ENSG00000222465 | ENSG00000154645 |
| ENSG00000117472 | ENSG00000110900 | ENSG00000091879 |
| ENSG00000241755 | ENSG00000179023 | ENSG00000156509 |
| ENSG00000271522 | ENSG00000185269 | ENSG00000277586 |
| ENSG00000183837 | ENSG00000238165 | ENSG00000279958 |
| ENSG00000270038 | ENSG00000156804 | ENSG00000232901 |
| ENSG00000211895 | ENSG00000279685 | ENSG00000237852 |
| ENSG00000170476 | ENSG00000140481 | ENSG00000246582 |
| ENSG00000196581 | ENSG00000162366 | ENSG00000158714 |
| ENSG00000227777 | ENSG00000281664 | ENSG00000266288 |
| ENSG00000104332 | ENSG00000235920 | ENSG00000229065 |
| ENSG00000196436 | ENSG00000073670 | ENSG00000220392 |
| ENSG00000106484 | ENSG00000227507 | ENSG00000233668 |
| ENSG00000037965 | ENSG00000279903 | ENSG00000151322 |
| ENSG00000124194 | ENSG00000240302 | ENSG00000120675 |
| ENSG00000250305 | ENSG00000168394 | ENSG00000175928 |
| ENSG00000139800 | ENSG00000143320 | ENSG00000225623 |
| ENSG00000123500 | ENSG00000161896 | ENSG00000111424 |
| ENSG00000135298 | ENSG00000153291 | ENSG00000234841 |
| ENSG00000165323 | ENSG00000182901 | ENSG00000277050 |
| ENSG00000253168 | ENSG00000100678 | ENSG00000154856 |
| ENSG00000154760 | ENSG00000196381 | ENSG00000166086 |
| ENSG00000139269 | ENSG00000183145 | ENSG00000184588 |
| ENSG00000273138 | ENSG00000137809 | ENSG00000278171 |
| ENSG00000244756 | ENSG00000122735 | ENSG00000082014 |
| ENSG00000108001 | ENSG00000188959 | ENSG00000272764 |
| ENSG00000176153 | ENSG00000221116 | ENSG00000211632 |
| ENSG00000166482 | ENSG00000170615 | ENSG00000141576 |
| ENSG00000110436 | ENSG00000145681 | ENSG00000240038 |
| ENSG00000260785 | ENSG00000183690 | ENSG00000278177 |
| ENSG00000235026 | ENSG00000136826 | ENSG00000170577 |
| ENSG00000255026 | ENSG00000259554 | ENSG00000274849 |
| ENSG00000229415 | ENSG00000081052 | ENSG00000167889 |
| ENSG00000154118 | ENSG00000264911 | ENSG00000268649 |
| ENSG00000089356 | ENSG00000101349 | ENSG00000258088 |
| ENSG00000198785 | ENSG00000278771 | ENSG00000136010 |
| ENSG00000101098 | ENSG00000233608 | ENSG00000125354 |
| ENSG00000103647 | ENSG00000270523 | ENSG00000226070 |
| ENSG00000206172 | ENSG00000214652 | ENSG00000270880 |
| ENSG00000271947 | ENSG00000198851 | ENSG00000231359 |
| ENSG00000167178 | ENSG00000214456 | ENSG00000180758 |
| ENSG00000013293 | ENSG00000236008 | ENSG00000267429 |
| ENSG00000157087 | ENSG00000254510 | ENSG00000227267 |
| ENSG00000274576 | ENSG00000222343 | ENSG00000279620 |
| ENSG00000228203 | ENSG00000223336 | ENSG00000214772 |
| ENSG00000277610 | ENSG00000271973 | ENSG00000150054 |
| ENSG00000269416 | ENSG00000165449 | ENSG00000259658 |
| ENSG00000273711 | ENSG00000103034 | ENSG00000131037 |
| ENSG00000225882 | ENSG00000119917 | ENSG00000272551 |
| ENSG00000254319 | ENSG00000261568 | ENSG00000149212 |
| ENSG00000189058 | ENSG00000103044 | ENSG00000231789 |
| ENSG00000197928 | ENSG00000166402 | ENSG00000273212 |
| ENSG00000165973 | ENSG00000272168 | ENSG00000214595 |
| ENSG00000145113 | ENSG00000108924 | ENSG00000147697 |
| ENSG00000188916 | ENSG00000103175 | ENSG00000258284 |
| ENSG00000211899 | ENSG00000198868 | ENSG00000165072 |
| ENSG00000211946 | ENSG00000255983 | ENSG00000103241 |
| ENSG00000090539 | ENSG00000260671 | ENSG00000162643 |
| ENSG00000184144 | ENSG00000010310 | ENSG00000172818 |
| ENSG00000176951 | ENSG00000231435 | ENSG00000260070 |
| ENSG00000160472 | ENSG00000238279 | ENSG00000121281 |
| ENSG00000074219 | ENSG00000279221 | ENSG00000272144 |
| ENSG00000187175 | ENSG00000279965 | ENSG00000258334 |
| ENSG00000136928 | ENSG00000262209 | ENSG00000102934 |
| ENSG00000168952 | ENSG00000176595 | ENSG00000143333 |
| ENSG00000196754 | ENSG00000111335 | ENSG00000224928 |
| ENSG00000162949 | ENSG00000256417 | ENSG00000225408 |
| ENSG00000231362 | ENSG00000114790 | ENSG00000273289 |
| ENSG00000130224 | ENSG00000284368 | ENSG00000048740 |
| ENSG00000133863 | ENSG00000203727 | ENSG00000235875 |
| ENSG00000132437 | ENSG00000105289 | ENSG00000112214 |
| ENSG00000280189 | ENSG00000114948 | ENSG00000246528 |
| ENSG00000272070 | ENSG00000115523 | ENSG00000160255 |
| ENSG00000104899 | ENSG00000115138 | ENSG00000279302 |
| ENSG00000165985 | ENSG00000188816 | ENSG00000140511 |
| ENSG00000172724 | ENSG00000186377 | ENSG00000185905 |
| ENSG00000138944 | ENSG00000238085 | ENSG00000254854 |
| ENSG00000086570 | ENSG00000172716 | ENSG00000250371 |
| ENSG00000171587 | ENSG00000277214 | ENSG00000154556 |
| ENSG00000236452 | ENSG00000273272 | ENSG00000102575 |
| ENSG00000165646 | ENSG00000268747 | ENSG00000186470 |
| ENSG00000118785 | ENSG00000234678 | ENSG00000156959 |
| ENSG00000139220 | ENSG00000277103 | ENSG00000230081 |
| ENSG00000102466 | ENSG00000229237 | ENSG00000067798 |
| ENSG00000204965 | ENSG00000124575 | ENSG00000172348 |
| ENSG00000152092 | ENSG00000138964 | ENSG00000224093 |
| ENSG00000251988 | ENSG00000162552 | ENSG00000277831 |
| ENSG00000174844 | ENSG00000267074 | ENSG00000175877 |
| ENSG00000228623 | ENSG00000209480 | ENSG00000172164 |
| ENSG00000154099 | ENSG00000197479 | ENSG00000006757 |
| ENSG00000226468 | ENSG00000184995 | ENSG00000103942 |
| ENSG00000188909 | ENSG00000178776 | ENSG00000113657 |
| ENSG00000230937 | ENSG00000248641 | ENSG00000277310 |
| ENSG00000276742 | ENSG00000134207 | ENSG00000144649 |
| ENSG00000267640 | ENSG00000241963 | ENSG00000168062 |
| ENSG00000164825 | ENSG00000116031 | ENSG00000075673 |
| ENSG00000227640 | ENSG00000283355 | ENSG00000198673 |
| ENSG00000274845 | ENSG00000180537 | ENSG00000229023 |
| ENSG00000211644 | ENSG00000112175 | ENSG00000198915 |
| ENSG00000206755 | ENSG00000125931 | ENSG00000231079 |
| ENSG00000009950 | ENSG00000165810 | ENSG00000139438 |
| ENSG00000188338 | ENSG00000127561 | ENSG00000269968 |
| ENSG00000145824 | ENSG00000283438 | ENSG00000188290 |
| ENSG00000169862 | ENSG00000253953 | ENSG00000234315 |
| ENSG00000173467 | ENSG00000267641 | ENSG00000160111 |
| ENSG00000186417 | ENSG00000254675 | ENSG00000278236 |
| ENSG00000106536 | ENSG00000199673 | ENSG00000167767 |
| ENSG00000248596 | ENSG00000218792 | ENSG00000017483 |
| ENSG00000230498 | ENSG00000142273 | ENSG00000206538 |
| ENSG00000279638 | ENSG00000120262 | ENSG00000111110 |
| ENSG00000006047 | ENSG00000198075 | ENSG00000100867 |
| ENSG00000160180 | ENSG00000259954 | ENSG00000235272 |
| ENSG00000197984 | ENSG00000129951 | ENSG00000042062 |
| ENSG00000189377 | ENSG00000135638 | ENSG00000120594 |
| ENSG00000161905 | ENSG00000114805 | ENSG00000217334 |
| ENSG00000101638 | ENSG00000183092 | ENSG00000259279 |
| ENSG00000254816 | ENSG00000198753 | ENSG00000254739 |
| ENSG00000189334 | ENSG00000125144 | ENSG00000270761 |
| ENSG00000238554 | ENSG00000137198 | ENSG00000113389 |
| ENSG00000187546 | ENSG00000177721 | ENSG00000270951 |
| ENSG00000197813 | ENSG00000273650 | ENSG00000134460 |
| ENSG00000271907 | ENSG00000148408 | ENSG00000134769 |
| ENSG00000241913 | ENSG00000129596 | ENSG00000261126 |
| ENSG00000198797 | ENSG00000124610 | ENSG00000139910 |
| ENSG00000132932 | ENSG00000113212 | ENSG00000163491 |
| ENSG00000278929 | ENSG00000257835 | ENSG00000270781 |
| ENSG00000260658 | ENSG00000176485 | ENSG00000068615 |
| ENSG00000251639 | ENSG00000140937 | ENSG00000253596 |
| ENSG00000256612 | ENSG00000249020 | ENSG00000003987 |
| ENSG00000169436 | ENSG00000115112 | ENSG00000236946 |
| ENSG00000221083 | ENSG00000170477 | ENSG00000279140 |
| ENSG00000211942 | ENSG00000244692 | ENSG00000179008 |
| ENSG00000276368 | ENSG00000139352 | ENSG00000100079 |
| ENSG00000105642 | ENSG00000113594 | ENSG00000174946 |
| ENSG00000270953 | ENSG00000100234 | ENSG00000238961 |
| ENSG00000206910 | ENSG00000134259 | ENSG00000099954 |
| ENSG00000131668 | ENSG00000168743 | ENSG00000179855 |
| ENSG00000112183 | ENSG00000170624 | ENSG00000160593 |
| ENSG00000228065 | ENSG00000111341 | ENSG00000157423 |
| ENSG00000163220 | ENSG00000166592 | ENSG00000122786 |
| ENSG00000265948 | ENSG00000269978 | ENSG00000240625 |
| ENSG00000149654 | ENSG00000041353 | ENSG00000256694 |
| ENSG00000094755 | ENSG00000188306 | ENSG00000170961 |
| ENSG00000060140 | ENSG00000277250 | ENSG00000272991 |
| ENSG00000178187 | ENSG00000171357 | ENSG00000263065 |
| ENSG00000100433 | ENSG00000267713 | ENSG00000267724 |
| ENSG00000206687 | ENSG00000077942 | ENSG00000163600 |
| ENSG00000113205 | ENSG00000207217 | ENSG00000081181 |
| ENSG00000118432 | ENSG00000265260 | ENSG00000154153 |
| ENSG00000173432 | ENSG00000124657 | ENSG00000254712 |
| ENSG00000120322 | ENSG00000197748 | ENSG00000280228 |
| ENSG00000132321 | ENSG00000196562 | ENSG00000135919 |
| ENSG00000107147 | ENSG00000139173 | ENSG00000106785 |
| ENSG00000242863 | ENSG00000117226 | ENSG00000007312 |
| ENSG00000225234 | ENSG00000240692 | ENSG00000204832 |
| ENSG00000019186 | ENSG00000262358 | ENSG00000116852 |
| ENSG00000183780 | ENSG00000261934 | ENSG00000283517 |
| ENSG00000254221 | ENSG00000170231 | ENSG00000271234 |
| ENSG00000169515 | ENSG00000279050 | ENSG00000147394 |
| ENSG00000214076 | ENSG00000248305 | ENSG00000119714 |
| ENSG00000200718 | ENSG00000258647 | ENSG00000225356 |
| ENSG00000278891 | ENSG00000254771 | ENSG00000172243 |
| ENSG00000243353 | ENSG00000147488 | ENSG00000227755 |
| ENSG00000188175 | ENSG00000158296 | ENSG00000271992 |
| ENSG00000245526 | ENSG00000267385 | ENSG00000168405 |
| ENSG00000262406 | ENSG00000163083 | ENSG00000250770 |
| ENSG00000226499 | ENSG00000077782 | ENSG00000277157 |
| ENSG00000197565 | ENSG00000225698 | ENSG00000204653 |
| ENSG00000273338 | ENSG00000101311 | ENSG00000164687 |
| ENSG00000169427 | ENSG00000264660 | ENSG00000261305 |
| ENSG00000122592 | ENSG00000119681 | ENSG00000174326 |
| ENSG00000160183 | ENSG00000226769 | ENSG00000181631 |
| ENSG00000162873 | ENSG00000258376 | ENSG00000251508 |
| ENSG00000054356 | ENSG00000225907 | ENSG00000123095 |
| ENSG00000169856 | ENSG00000231546 | ENSG00000258872 |
| ENSG00000127324 | ENSG00000134138 | ENSG00000260244 |
| ENSG00000106278 | ENSG00000187210 | ENSG00000279166 |
| ENSG00000277653 | ENSG00000122861 | ENSG00000281706 |
| ENSG00000276786 | ENSG00000079101 | ENSG00000110799 |
| ENSG00000009694 | ENSG00000163898 | ENSG00000154146 |
| ENSG00000268117 | ENSG00000128849 | ENSG00000268520 |
| ENSG00000259240 | ENSG00000201616 | ENSG00000120217 |
| ENSG00000126803 | ENSG00000135605 | ENSG00000145623 |
| ENSG00000175206 | ENSG00000233551 | ENSG00000050030 |
| ENSG00000175497 | ENSG00000276085 | ENSG00000262979 |
| ENSG00000212529 | ENSG00000185745 | ENSG00000106560 |
| ENSG00000253985 | ENSG00000127928 | ENSG00000007306 |
| ENSG00000171786 | ENSG00000242134 | ENSG00000185031 |
| ENSG00000226857 | ENSG00000233844 | ENSG00000186235 |
| ENSG00000188833 | ENSG00000167748 | ENSG00000240527 |
| ENSG00000006377 | ENSG00000121905 | ENSG00000196155 |
| ENSG00000269989 | ENSG00000196126 | ENSG00000213560 |
| ENSG00000272674 | ENSG00000100290 | ENSG00000231160 |
| ENSG00000145423 | ENSG00000132329 | ENSG00000236263 |
| ENSG00000113209 | ENSG00000177380 | ENSG00000227200 |
| ENSG00000007372 | ENSG00000223039 | ENSG00000267215 |
| ENSG00000137959 | ENSG00000175356 | ENSG00000275569 |
| ENSG00000228014 | ENSG00000280294 | ENSG00000120875 |
| ENSG00000197705 | ENSG00000279949 | ENSG00000226709 |
| ENSG00000225477 | ENSG00000239912 | ENSG00000139626 |
| ENSG00000261786 | ENSG00000153885 | ENSG00000241693 |
| ENSG00000144460 | ENSG00000049130 | ENSG00000220600 |
| ENSG00000012223 | ENSG00000274444 | ENSG00000225706 |
| ENSG00000252473 | ENSG00000283199 | ENSG00000132563 |
| ENSG00000198483 | ENSG00000184058 | ENSG00000182853 |
| ENSG00000168772 | ENSG00000260015 | ENSG00000156113 |
| ENSG00000152611 | ENSG00000279044 | ENSG00000100344 |
| ENSG00000151892 | ENSG00000115290 | ENSG00000110079 |
| ENSG00000133048 | ENSG00000221643 | ENSG00000235024 |
| ENSG00000284613 | ENSG00000269886 | ENSG00000137203 |
| ENSG00000116254 | ENSG00000242076 | ENSG00000231705 |
| ENSG00000137699 | ENSG00000175264 | ENSG00000262089 |
| ENSG00000136099 | ENSG00000139973 | ENSG00000131711 |
| ENSG00000237645 | ENSG00000239923 | ENSG00000233492 |
| ENSG00000200176 | ENSG00000196542 | ENSG00000253430 |
| ENSG00000196724 | ENSG00000215802 | ENSG00000172346 |
| ENSG00000240382 | ENSG00000115556 | ENSG00000254281 |
| ENSG00000135324 | ENSG00000258986 | ENSG00000175414 |
| ENSG00000047457 | ENSG00000278865 | ENSG00000213199 |
| ENSG00000268105 | ENSG00000185306 | ENSG00000198821 |
| ENSG00000101680 | ENSG00000117122 | ENSG00000230310 |
| ENSG00000252982 | ENSG00000186567 | ENSG00000203279 |
| ENSG00000196167 | ENSG00000204642 | ENSG00000264275 |
| ENSG00000170017 | ENSG00000171119 | ENSG00000251611 |
| ENSG00000174776 | ENSG00000251583 | ENSG00000247095 |
| ENSG00000132938 | ENSG00000125968 | ENSG00000151883 |
| ENSG00000236699 | ENSG00000279885 | ENSG00000165215 |
| ENSG00000150551 | ENSG00000267201 | ENSG00000271259 |
| ENSG00000157064 | ENSG00000092051 | ENSG00000231703 |
| ENSG00000124493 | ENSG00000269246 | ENSG00000155307 |
| ENSG00000144218 | ENSG00000196277 | ENSG00000170962 |
| ENSG00000262482 | ENSG00000134532 | ENSG00000269834 |
| ENSG00000137745 | ENSG00000171345 | ENSG00000118985 |
| ENSG00000243238 | ENSG00000143147 | ENSG00000184347 |
| ENSG00000273301 | ENSG00000231134 | ENSG00000272440 |
| ENSG00000207279 | ENSG00000249092 | ENSG00000273557 |
| ENSG00000187122 | ENSG00000226711 | ENSG00000242553 |
| ENSG00000242173 | ENSG00000089199 | ENSG00000187475 |
| ENSG00000248266 | ENSG00000138696 | ENSG00000200673 |
| ENSG00000109956 | ENSG00000185275 | ENSG00000231787 |
| ENSG00000008226 | ENSG00000089558 | ENSG00000267795 |
| ENSG00000089250 | ENSG00000272482 | ENSG00000072858 |
| ENSG00000225792 | ENSG00000201600 | ENSG00000182674 |
| ENSG00000165272 | ENSG00000176788 | ENSG00000272264 |
| ENSG00000204175 | ENSG00000015520 | ENSG00000236457 |
| ENSG00000143195 | ENSG00000251697 | ENSG00000229391 |
| ENSG00000260385 | ENSG00000167676 | ENSG00000050165 |
| ENSG00000239122 | ENSG00000144837 | ENSG00000005249 |
| ENSG00000272479 | ENSG00000144452 | ENSG00000240583 |
| ENSG00000157890 | ENSG00000129009 | ENSG00000173530 |
| ENSG00000164929 | ENSG00000272505 | ENSG00000267284 |
| ENSG00000207110 | ENSG00000261105 | ENSG00000228709 |
| ENSG00000163739 | ENSG00000274213 | ENSG00000168386 |
| ENSG00000175785 | ENSG00000249057 | ENSG00000158077 |
| ENSG00000022556 | ENSG00000147168 | ENSG00000243444 |
| ENSG00000156103 | ENSG00000229299 | ENSG00000225163 |
| ENSG00000137673 | ENSG00000259740 | ENSG00000090661 |
| ENSG00000211625 | ENSG00000257880 | ENSG00000262434 |
| ENSG00000183778 | ENSG00000166206 | ENSG00000254363 |
| ENSG00000133067 | ENSG00000095397 | ENSG00000273524 |
| ENSG00000080031 | ENSG00000173947 | ENSG00000262533 |
| ENSG00000259757 | ENSG00000144834 | ENSG00000268601 |
| ENSG00000114757 | ENSG00000113356 | ENSG00000171303 |
| ENSG00000251546 | ENSG00000256762 | ENSG00000221571 |
| ENSG00000272330 | ENSG00000272088 | ENSG00000164430 |
| ENSG00000258927 | ENSG00000202125 | ENSG00000064042 |
| ENSG00000101180 | ENSG00000255160 | ENSG00000263934 |
| ENSG00000146411 | ENSG00000170786 | ENSG00000240240 |
| ENSG00000223350 | ENSG00000161133 | ENSG00000279013 |
| ENSG00000124935 | ENSG00000178662 | ENSG00000277144 |
| ENSG00000254877 | ENSG00000114279 | ENSG00000254027 |
| ENSG00000123201 | ENSG00000279952 | ENSG00000204472 |
| ENSG00000185290 | ENSG00000179750 | ENSG00000204261 |
| ENSG00000148600 | ENSG00000197921 | ENSG00000266610 |
| ENSG00000166105 | ENSG00000166106 | ENSG00000270381 |
| ENSG00000228330 | ENSG00000120324 | ENSG00000137462 |
| ENSG00000258405 | ENSG00000132259 | ENSG00000277373 |
| ENSG00000042980 | ENSG00000267231 | ENSG00000105609 |
| ENSG00000205038 | ENSG00000268375 | ENSG00000256299 |
| ENSG00000276105 | ENSG00000207175 | ENSG00000249493 |
| ENSG00000257178 | ENSG00000228869 | ENSG00000120162 |
| ENSG00000204956 | ENSG00000227811 | ENSG00000273958 |
| ENSG00000229807 | ENSG00000125618 | ENSG00000276855 |
| ENSG00000160401 | ENSG00000149451 | ENSG00000100504 |
| ENSG00000052344 | ENSG00000196684 | ENSG00000228569 |
| ENSG00000204065 | ENSG00000109794 | ENSG00000244300 |
| ENSG00000170324 | ENSG00000162078 | ENSG00000076356 |
| ENSG00000133636 | ENSG00000278588 | ENSG00000185811 |
| ENSG00000206075 | ENSG00000065833 | ENSG00000282639 |
| ENSG00000157445 | ENSG00000239783 | ENSG00000279712 |
| ENSG00000007516 | ENSG00000256443 | ENSG00000279673 |
| ENSG00000261426 | ENSG00000157601 | ENSG00000225183 |
| ENSG00000169435 | ENSG00000201289 | ENSG00000181392 |
| ENSG00000158125 | ENSG00000075223 | ENSG00000199347 |
| ENSG00000114638 | ENSG00000263969 | ENSG00000110448 |
| ENSG00000156234 | ENSG00000078596 | ENSG00000262362 |
| ENSG00000213877 | ENSG00000283366 | ENSG00000007866 |
| ENSG00000232000 | ENSG00000205045 | ENSG00000245164 |
| ENSG00000147257 | ENSG00000240474 | ENSG00000277978 |
| ENSG00000274080 | ENSG00000163568 | ENSG00000277602 |
| ENSG00000128536 | ENSG00000150722 | ENSG00000146477 |
| ENSG00000139292 | ENSG00000174171 | ENSG00000109684 |
| ENSG00000197584 | ENSG00000004468 | ENSG00000175463 |
| ENSG00000207468 | ENSG00000147234 | ENSG00000263847 |
| ENSG00000261431 | ENSG00000272279 | ENSG00000235907 |
| ENSG00000197641 | ENSG00000185739 | ENSG00000256274 |
| ENSG00000199932 | ENSG00000102935 | ENSG00000139289 |
| ENSG00000147869 | ENSG00000168077 | ENSG00000171016 |
| ENSG00000279340 | ENSG00000168502 | ENSG00000203618 |
| ENSG00000167653 | ENSG00000132530 | ENSG00000139537 |
| ENSG00000134762 | ENSG00000104415 | ENSG00000274895 |
| ENSG00000226453 | ENSG00000258827 | ENSG00000281398 |
| ENSG00000240409 | ENSG00000196154 | ENSG00000234093 |
| ENSG00000260412 | ENSG00000073849 | ENSG00000273979 |
| ENSG00000279747 | ENSG00000023445 | ENSG00000263335 |
| ENSG00000273853 | ENSG00000128709 | ENSG00000213976 |
| ENSG00000264943 | ENSG00000107796 | ENSG00000203585 |
| ENSG00000134363 | ENSG00000145536 | ENSG00000157927 |
| ENSG00000154874 | ENSG00000248791 | ENSG00000213363 |
| ENSG00000242516 | ENSG00000239333 | ENSG00000089820 |
| ENSG00000078295 | ENSG00000250574 | ENSG00000261430 |
| ENSG00000280098 | ENSG00000106624 | ENSG00000243607 |
| ENSG00000112232 | ENSG00000112715 | ENSG00000254726 |
| ENSG00000235688 | ENSG00000007933 | ENSG00000109472 |
| ENSG00000212933 | ENSG00000177685 | ENSG00000164742 |
| ENSG00000183044 | ENSG00000251163 | ENSG00000179431 |
| ENSG00000233101 | ENSG00000154165 | ENSG00000203499 |
| ENSG00000171812 | ENSG00000254331 | ENSG00000113739 |
| ENSG00000198502 | ENSG00000092096 | ENSG00000269397 |
| ENSG00000198542 | ENSG00000152154 | ENSG00000243449 |
| ENSG00000261330 | ENSG00000160460 | ENSG00000107014 |
| ENSG00000262801 | ENSG00000270876 | ENSG00000186231 |
| ENSG00000241354 | ENSG00000097096 | ENSG00000229668 |
| ENSG00000206698 | ENSG00000241905 | ENSG00000259925 |
| ENSG00000269516 | ENSG00000282221 | ENSG00000149418 |
| ENSG00000157680 | ENSG00000170271 | ENSG00000279176 |
| ENSG00000162630 | ENSG00000182379 | ENSG00000240211 |
| ENSG00000242371 | ENSG00000229754 | ENSG00000235051 |
| ENSG00000262621 | ENSG00000145284 | ENSG00000271596 |
| ENSG00000169432 | ENSG00000280265 | ENSG00000273763 |
| ENSG00000177519 | ENSG00000072818 | ENSG00000136040 |
| ENSG00000144355 | ENSG00000133083 | ENSG00000235449 |
| ENSG00000130513 | ENSG00000229014 | ENSG00000113248 |
| ENSG00000227291 | ENSG00000274135 | ENSG00000272175 |
| ENSG00000165092 | ENSG00000170396 | ENSG00000278937 |
| ENSG00000250343 | ENSG00000100842 | ENSG00000069399 |
| ENSG00000106341 | ENSG00000120051 | ENSG00000218690 |
| ENSG00000202374 | ENSG00000143341 | ENSG00000167619 |
| ENSG00000242381 | ENSG00000258624 | ENSG00000273792 |
| ENSG00000161249 | ENSG00000162591 | ENSG00000239822 |
| ENSG00000112139 | ENSG00000198157 | ENSG00000123338 |
| ENSG00000254049 | ENSG00000275611 | ENSG00000214124 |
| ENSG00000213785 | ENSG00000237721 | ENSG00000239713 |
| ENSG00000278957 | ENSG00000249526 | ENSG00000141337 |
| ENSG00000021645 | ENSG00000176971 | ENSG00000266385 |
| ENSG00000123836 | ENSG00000164694 | ENSG00000260193 |
| ENSG00000136267 | ENSG00000241829 | ENSG00000273920 |
| ENSG00000126778 | ENSG00000244356 | ENSG00000275476 |
| ENSG00000187984 | ENSG00000205517 | ENSG00000219150 |
| ENSG00000112936 | ENSG00000230798 | ENSG00000241357 |
| ENSG00000170382 | ENSG00000170891 | ENSG00000135318 |
| ENSG00000108375 | ENSG00000168785 | ENSG00000244694 |
| ENSG00000112164 | ENSG00000025708 | ENSG00000179144 |
| ENSG00000283108 | ENSG00000167654 | ENSG00000187068 |
| ENSG00000117971 | ENSG00000226644 | ENSG00000064309 |
| ENSG00000105549 | ENSG00000221955 | ENSG00000163009 |
| ENSG00000110777 | ENSG00000115956 | ENSG00000204241 |
| ENSG00000120057 | ENSG00000223111 | ENSG00000196274 |
| ENSG00000149054 | ENSG00000242614 | ENSG00000181404 |
| ENSG00000266549 | ENSG00000174175 | ENSG00000018280 |
| ENSG00000171346 | ENSG00000232759 | ENSG00000272057 |
| ENSG00000260230 | ENSG00000278011 | ENSG00000049323 |
| ENSG00000163751 | ENSG00000069535 | ENSG00000274605 |
| ENSG00000103534 | ENSG00000212978 | ENSG00000085117 |
| ENSG00000124102 | ENSG00000124126 | ENSG00000230415 |
| ENSG00000219159 | ENSG00000280176 | ENSG00000111057 |
| ENSG00000239855 | ENSG00000130176 | ENSG00000118523 |
| ENSG00000198807 | ENSG00000256824 | ENSG00000266237 |
| ENSG00000166257 | ENSG00000180801 | ENSG00000223375 |
| ENSG00000198353 | ENSG00000188596 | ENSG00000074590 |
| ENSG00000267886 | ENSG00000254815 | ENSG00000186998 |
| ENSG00000100170 | ENSG00000149968 | ENSG00000261618 |
| ENSG00000169064 | ENSG00000274934 | ENSG00000133138 |
| ENSG00000235961 | ENSG00000135111 | ENSG00000146955 |
| ENSG00000135824 | ENSG00000240775 | ENSG00000136717 |
| ENSG00000211448 | ENSG00000228696 | ENSG00000266222 |
| ENSG00000139970 | ENSG00000229970 | ENSG00000257743 |
| ENSG00000281128 | ENSG00000186212 | ENSG00000183598 |
| ENSG00000152580 | ENSG00000248682 | ENSG00000204267 |
| ENSG00000141579 | ENSG00000272695 | ENSG00000198756 |
| ENSG00000207322 | ENSG00000259404 | ENSG00000278276 |
| ENSG00000088992 | ENSG00000273153 | ENSG00000271938 |
| ENSG00000230316 | ENSG00000183196 | ENSG00000090932 |
| ENSG00000248117 | ENSG00000221890 | ENSG00000274253 |
| ENSG00000154678 | ENSG00000101134 | ENSG00000138722 |
| ENSG00000029559 | ENSG00000196616 | ENSG00000253404 |
| ENSG00000186919 | ENSG00000164920 | ENSG00000228431 |
| ENSG00000250360 | ENSG00000165568 | ENSG00000113721 |
| ENSG00000131203 | ENSG00000151067 | ENSG00000261226 |
| ENSG00000254802 | ENSG00000254141 | ENSG00000232034 |
| ENSG00000234026 | ENSG00000236556 | ENSG00000228115 |
| ENSG00000234715 | ENSG00000255417 | ENSG00000241106 |
| ENSG00000100985 | ENSG00000164082 | ENSG00000239552 |
| ENSG00000280406 | ENSG00000115423 | ENSG00000128655 |
| ENSG00000183914 | ENSG00000187955 | ENSG00000235531 |
| ENSG00000088882 | ENSG00000105427 | ENSG00000170629 |
| ENSG00000060718 | ENSG00000272788 | ENSG00000227535 |
| ENSG00000105707 | ENSG00000200903 | ENSG00000207205 |
| ENSG00000242265 | ENSG00000225643 | ENSG00000164318 |
| ENSG00000163873 | ENSG00000173320 | ENSG00000254777 |
| ENSG00000274750 | ENSG00000148219 | ENSG00000145431 |
| ENSG00000241418 | ENSG00000254343 | ENSG00000187037 |
| ENSG00000173917 | ENSG00000267568 | ENSG00000229875 |
| ENSG00000019505 | ENSG00000258982 | ENSG00000225195 |
| ENSG00000174502 | ENSG00000266569 | ENSG00000226781 |
| ENSG00000184368 | ENSG00000268475 | ENSG00000251456 |
| ENSG00000233756 | ENSG00000054598 | ENSG00000010610 |
| ENSG00000231153 | ENSG00000230327 | ENSG00000161281 |
| ENSG00000151136 | ENSG00000079102 | ENSG00000283757 |
| ENSG00000142449 | ENSG00000201542 | ENSG00000101298 |
| ENSG00000161798 | ENSG00000128596 | ENSG00000269918 |
| ENSG00000117152 | ENSG00000225479 | ENSG00000153495 |
| ENSG00000226508 | ENSG00000249684 | ENSG00000261970 |
| ENSG00000255575 | ENSG00000225662 | ENSG00000242818 |
| ENSG00000147041 | ENSG00000197142 | ENSG00000212607 |
| ENSG00000002933 | ENSG00000106006 | ENSG00000138738 |
| ENSG00000182771 | ENSG00000074047 | ENSG00000148908 |
| ENSG00000185974 | ENSG00000229567 | ENSG00000282122 |
| ENSG00000169760 | ENSG00000135046 | ENSG00000278709 |
| ENSG00000137857 | ENSG00000258083 | ENSG00000225492 |
| ENSG00000277526 | ENSG00000260293 | ENSG00000182648 |
| ENSG00000110693 | ENSG00000231255 | ENSG00000278546 |
| ENSG00000165949 | ENSG00000187730 | ENSG00000099998 |
| ENSG00000188620 | ENSG00000266578 | ENSG00000196187 |
| ENSG00000122367 | ENSG00000163075 | ENSG00000236532 |
| ENSG00000077327 | ENSG00000227368 | ENSG00000111452 |
| ENSG00000204385 | ENSG00000253230 | ENSG00000255003 |
| ENSG00000165164 | ENSG00000198879 | ENSG00000141338 |
| ENSG00000230910 | ENSG00000225849 | ENSG00000272130 |
| ENSG00000100095 | ENSG00000106789 | ENSG00000255374 |
| ENSG00000139329 | ENSG00000005961 | ENSG00000213076 |
| ENSG00000196376 | ENSG00000236794 | ENSG00000102385 |
| ENSG00000204711 | ENSG00000204389 | ENSG00000075618 |
| ENSG00000146360 | ENSG00000176900 | ENSG00000182871 |
| ENSG00000257520 | ENSG00000273112 | ENSG00000138685 |
| ENSG00000275710 | ENSG00000124731 | ENSG00000254087 |
| ENSG00000172461 | ENSG00000262503 | ENSG00000243659 |
| ENSG00000114204 | ENSG00000125538 | ENSG00000242810 |
| ENSG00000185686 | ENSG00000160188 | ENSG00000173083 |
| ENSG00000280336 | ENSG00000177738 | ENSG00000134531 |
| ENSG00000124785 | ENSG00000279427 | ENSG00000279752 |
| ENSG00000203685 | ENSG00000274370 | ENSG00000254887 |
| ENSG00000244757 | ENSG00000181031 | ENSG00000163364 |
| ENSG00000144847 | ENSG00000280344 | ENSG00000177508 |
| ENSG00000013588 | ENSG00000133687 | ENSG00000111913 |
| ENSG00000198732 | ENSG00000119125 | ENSG00000103522 |
| ENSG00000076770 | ENSG00000279197 | ENSG00000263812 |
| ENSG00000235548 | ENSG00000162105 | ENSG00000135643 |
| ENSG00000224271 | ENSG00000269421 | ENSG00000153446 |
| ENSG00000258544 | ENSG00000213145 | ENSG00000272071 |
| ENSG00000110148 | ENSG00000164175 | ENSG00000241243 |
| ENSG00000172159 | ENSG00000182752 | ENSG00000050438 |
| ENSG00000163347 | ENSG00000140379 | ENSG00000186891 |
| ENSG00000146216 | ENSG00000242756 | ENSG00000109610 |
| ENSG00000228340 | ENSG00000253764 | ENSG00000019102 |
| ENSG00000184486 | ENSG00000106069 | ENSG00000011028 |
| ENSG00000179542 | ENSG00000235098 | ENSG00000100979 |
| ENSG00000080493 | ENSG00000253339 | ENSG00000243701 |
| ENSG00000229770 | ENSG00000156042 | ENSG00000118004 |
| ENSG00000104892 | ENSG00000188050 | ENSG00000235111 |
| ENSG00000148942 | ENSG00000162068 | ENSG00000115295 |
| ENSG00000207063 | ENSG00000223694 | ENSG00000255232 |
| ENSG00000067715 | ENSG00000176222 | ENSG00000186150 |
| ENSG00000095713 | ENSG00000164674 | ENSG00000087842 |
| ENSG00000106483 | ENSG00000221102 | ENSG00000105792 |
| ENSG00000152495 | ENSG00000267259 | ENSG00000095970 |
| ENSG00000226320 | ENSG00000263432 | ENSG00000132854 |
| ENSG00000058866 | ENSG00000136167 | ENSG00000256448 |
| ENSG00000070526 | ENSG00000270076 | ENSG00000229109 |
| ENSG00000156687 | ENSG00000169031 | ENSG00000110660 |
| ENSG00000279701 | ENSG00000179909 | ENSG00000125571 |
| ENSG00000167858 | ENSG00000255370 | ENSG00000228221 |
| ENSG00000175093 | ENSG00000227403 | ENSG00000163453 |
| ENSG00000176840 | ENSG00000113296 | ENSG00000279742 |
| ENSG00000225756 | ENSG00000218537 | ENSG00000120658 |
| ENSG00000250682 | ENSG00000184925 | ENSG00000275070 |
| ENSG00000197057 | ENSG00000185052 | ENSG00000260641 |
| ENSG00000226383 | ENSG00000103485 | ENSG00000186976 |
| ENSG00000109906 | ENSG00000256137 | ENSG00000225206 |
| ENSG00000164099 | ENSG00000242986 | ENSG00000074706 |
| ENSG00000275385 | ENSG00000053108 | ENSG00000198846 |
| ENSG00000270742 | ENSG00000234523 | ENSG00000163485 |
| ENSG00000188883 | ENSG00000201201 | ENSG00000263528 |
| ENSG00000011465 | ENSG00000142611 | ENSG00000073792 |
| ENSG00000172733 | ENSG00000256040 | ENSG00000160191 |
| ENSG00000206337 | ENSG00000148344 | ENSG00000175697 |
| ENSG00000132470 | ENSG00000134775 | ENSG00000248636 |
| ENSG00000128710 | ENSG00000165457 | ENSG00000281538 |
| ENSG00000112539 | ENSG00000165124 | ENSG00000100097 |
| ENSG00000125730 | ENSG00000183098 | ENSG00000198682 |
| ENSG00000201944 | ENSG00000223345 | ENSG00000186197 |
| ENSG00000268036 | ENSG00000102287 | ENSG00000128917 |
| ENSG00000163331 | ENSG00000239257 | ENSG00000230001 |
| ENSG00000183850 | ENSG00000242807 | ENSG00000149294 |
| ENSG00000072041 | ENSG00000232474 | ENSG00000152402 |
| ENSG00000106511 | ENSG00000133401 | ENSG00000218986 |
| ENSG00000274718 | ENSG00000260640 | ENSG00000187905 |
| ENSG00000167964 | ENSG00000163393 | ENSG00000113083 |
| ENSG00000232396 | ENSG00000243024 | ENSG00000145908 |
| ENSG00000151490 | ENSG00000242979 | ENSG00000251254 |
| ENSG00000150394 | ENSG00000197291 | ENSG00000255165 |
| ENSG00000178750 | ENSG00000084453 | ENSG00000239774 |
| ENSG00000199370 | ENSG00000144152 | ENSG00000237402 |
| ENSG00000178401 | ENSG00000263574 | ENSG00000255780 |
| ENSG00000257239 | ENSG00000137474 | ENSG00000251034 |
| ENSG00000124440 | ENSG00000108830 | ENSG00000241959 |
| ENSG00000177511 | ENSG00000093134 | ENSG00000123191 |
| ENSG00000231566 | ENSG00000159166 | ENSG00000240509 |
| ENSG00000161298 | ENSG00000166741 | ENSG00000137266 |
| ENSG00000186310 | ENSG00000167476 | ENSG00000272382 |
| ENSG00000281508 | ENSG00000147100 | ENSG00000211645 |
| ENSG00000239831 | ENSG00000174640 | ENSG00000226363 |
| ENSG00000133392 | ENSG00000184608 | ENSG00000154736 |
| ENSG00000179603 | ENSG00000279829 | ENSG00000106853 |
| ENSG00000170835 | ENSG00000236333 | ENSG00000267058 |
| ENSG00000196549 | ENSG00000144191 | ENSG00000251379 |
| ENSG00000144366 | ENSG00000132274 | ENSG00000163564 |
| ENSG00000275965 | ENSG00000258913 | ENSG00000048052 |
| ENSG00000090382 | ENSG00000167371 | ENSG00000008516 |
| ENSG00000274055 | ENSG00000176895 | ENSG00000274536 |
| ENSG00000115221 | ENSG00000278055 | ENSG00000231300 |
| ENSG00000284648 | ENSG00000229891 | ENSG00000279814 |
| ENSG00000081138 | ENSG00000139926 | ENSG00000272990 |
| ENSG00000243955 | ENSG00000147862 | ENSG00000168672 |
| ENSG00000183844 | ENSG00000105711 | ENSG00000259723 |
| ENSG00000154127 | ENSG00000255399 | ENSG00000159588 |
| ENSG00000222276 | ENSG00000066248 | ENSG00000278733 |
| ENSG00000243642 | ENSG00000105467 | ENSG00000233448 |
| ENSG00000276740 | ENSG00000189410 | ENSG00000231752 |
| ENSG00000228807 | ENSG00000255240 | ENSG00000103460 |
| ENSG00000186847 | ENSG00000235109 | ENSG00000230735 |
| ENSG00000110975 | ENSG00000121753 | ENSG00000227064 |
| ENSG00000049089 | ENSG00000137727 | ENSG00000112981 |
| ENSG00000207118 | ENSG00000206585 | ENSG00000267196 |
| ENSG00000253520 | ENSG00000180828 | ENSG00000237818 |
| ENSG00000140873 | ENSG00000273219 | ENSG00000270000 |
| ENSG00000237978 | ENSG00000077984 | ENSG00000268266 |
| ENSG00000259439 | ENSG00000240412 | ENSG00000185633 |
| ENSG00000183066 | ENSG00000180190 | ENSG00000283415 |
| ENSG00000154319 | ENSG00000137672 | ENSG00000258545 |
| ENSG00000162687 | ENSG00000141404 | ENSG00000180071 |
| ENSG00000166250 | ENSG00000198744 | ENSG00000244501 |
| ENSG00000221947 | ENSG00000167157 | ENSG00000239437 |
| ENSG00000187889 | ENSG00000260095 | ENSG00000204252 |
| ENSG00000101115 | ENSG00000278925 | ENSG00000071205 |
| ENSG00000181577 | ENSG00000002587 | ENSG00000115594 |
| ENSG00000053747 | ENSG00000260025 | ENSG00000273297 |
| ENSG00000202380 | ENSG00000119946 | ENSG00000166510 |
| ENSG00000227726 | ENSG00000249007 | ENSG00000281849 |
| ENSG00000283235 | ENSG00000258673 | ENSG00000253190 |
| ENSG00000229661 | ENSG00000129437 | ENSG00000232411 |
| ENSG00000183793 | ENSG00000178814 | ENSG00000276261 |
| ENSG00000187957 | ENSG00000204262 | ENSG00000261462 |
| ENSG00000272405 | ENSG00000235279 | ENSG00000273403 |
| ENSG00000206052 | ENSG00000265733 | ENSG00000143847 |
| ENSG00000282859 | ENSG00000236888 | ENSG00000123243 |
| ENSG00000179344 | ENSG00000255396 | ENSG00000255052 |
| ENSG00000120885 | ENSG00000271930 | ENSG00000227911 |
| ENSG00000154080 | ENSG00000279806 | ENSG00000127585 |
| ENSG00000105369 | ENSG00000275227 | ENSG00000239722 |
| ENSG00000083814 | ENSG00000203697 | ENSG00000167968 |
| ENSG00000188171 | ENSG00000264019 | ENSG00000137491 |
| ENSG00000177182 | ENSG00000168060 | ENSG00000172543 |
| ENSG00000233834 | ENSG00000204950 | ENSG00000278147 |
| ENSG00000184828 | ENSG00000120328 | ENSG00000268677 |
| ENSG00000234476 | ENSG00000270704 | ENSG00000110848 |
| ENSG00000239194 | ENSG00000103187 | ENSG00000276903 |
| ENSG00000183160 | ENSG00000233063 | ENSG00000278997 |
| ENSG00000175426 | ENSG00000181458 | ENSG00000126860 |
| ENSG00000279793 | ENSG00000105696 | ENSG00000053918 |
| ENSG00000172020 | ENSG00000170345 | ENSG00000261220 |
| ENSG00000242082 | ENSG00000081803 | ENSG00000240005 |
| ENSG00000230061 | ENSG00000000971 | ENSG00000251869 |
| ENSG00000126259 | ENSG00000105409 | ENSG00000214870 |
| ENSG00000077264 | ENSG00000240225 | ENSG00000141622 |
| ENSG00000165474 | ENSG00000158164 | ENSG00000275645 |
| ENSG00000116329 | ENSG00000108602 | ENSG00000146233 |
| ENSG00000003989 | ENSG00000116785 | ENSG00000203706 |
| ENSG00000256271 | ENSG00000121064 | ENSG00000277233 |
| ENSG00000225898 | ENSG00000284672 | ENSG00000258742 |
| ENSG00000249885 | ENSG00000254839 | ENSG00000064218 |
| ENSG00000231272 | ENSG00000154655 | ENSG00000177706 |
| ENSG00000151882 | ENSG00000198959 | ENSG00000256006 |
| ENSG00000182742 | ENSG00000241346 | ENSG00000196954 |
| ENSG00000260220 | ENSG00000160654 | ENSG00000221044 |
| ENSG00000065618 | ENSG00000166111 | ENSG00000134516 |
| ENSG00000215458 | ENSG00000263904 | ENSG00000156427 |
| ENSG00000211967 | ENSG00000237065 | ENSG00000271265 |
| ENSG00000274373 | ENSG00000267319 | ENSG00000184302 |
| ENSG00000276015 | ENSG00000256146 | ENSG00000283338 |
| ENSG00000176907 | ENSG00000272533 | ENSG00000198088 |
| ENSG00000162040 | ENSG00000167771 | ENSG00000240204 |
| ENSG00000137648 | ENSG00000242583 | ENSG00000267436 |
| ENSG00000178965 | ENSG00000170412 | ENSG00000231729 |
| ENSG00000185860 | ENSG00000279602 | ENSG00000279372 |
| ENSG00000111962 | ENSG00000169618 | ENSG00000280202 |
| ENSG00000128573 | ENSG00000260008 | ENSG00000280134 |
| ENSG00000280044 | ENSG00000271608 | ENSG00000252233 |
| ENSG00000196208 | ENSG00000267383 | ENSG00000050327 |
| ENSG00000270112 | ENSG00000222942 | ENSG00000252577 |
| ENSG00000272707 | ENSG00000177409 | ENSG00000249167 |
| ENSG00000233473 | ENSG00000153012 | ENSG00000122420 |
| ENSG00000135406 | ENSG00000171885 | ENSG00000237264 |
| ENSG00000005421 | ENSG00000273295 | ENSG00000236709 |
| ENSG00000164708 | ENSG00000258066 | ENSG00000234928 |
| ENSG00000163499 | ENSG00000139971 | ENSG00000200913 |
| ENSG00000199753 | ENSG00000164692 | ENSG00000273855 |
| ENSG00000115648 | ENSG00000137801 | ENSG00000187556 |
| ENSG00000163817 | ENSG00000064655 | ENSG00000216642 |
| ENSG00000068078 | ENSG00000162374 | ENSG00000251107 |
| ENSG00000224864 | ENSG00000019582 | ENSG00000018625 |
| ENSG00000272114 | ENSG00000241834 | ENSG00000277481 |
| ENSG00000175164 | ENSG00000110427 | ENSG00000132746 |
| ENSG00000106565 | ENSG00000128045 | ENSG00000133574 |
| ENSG00000200169 | ENSG00000230935 | ENSG00000279679 |
| ENSG00000176925 | ENSG00000088827 | ENSG00000254727 |
| ENSG00000158258 | ENSG00000240929 | ENSG00000255330 |
| ENSG00000138083 | ENSG00000131055 | ENSG00000072954 |
| ENSG00000183831 | ENSG00000165409 | ENSG00000082684 |
| ENSG00000184524 | ENSG00000214914 | ENSG00000230077 |
| ENSG00000271788 | ENSG00000162545 | ENSG00000188641 |
| ENSG00000180287 | ENSG00000274309 | ENSG00000233487 |
| ENSG00000206622 | ENSG00000112773 | ENSG00000167992 |
| ENSG00000278384 | ENSG00000254373 | ENSG00000242574 |
| ENSG00000100604 | ENSG00000168350 | ENSG00000261318 |
| ENSG00000214988 | ENSG00000275981 | ENSG00000121769 |
| ENSG00000131094 | ENSG00000258632 | ENSG00000179776 |
| ENSG00000129654 | ENSG00000167644 | ENSG00000240663 |
| ENSG00000279276 | ENSG00000142173 | ENSG00000137460 |
| ENSG00000196169 | ENSG00000259392 | ENSG00000118507 |
| ENSG00000152932 | ENSG00000040608 | ENSG00000133477 |
| ENSG00000130707 | ENSG00000237226 | ENSG00000161243 |
| ENSG00000175161 | ENSG00000196890 | ENSG00000165905 |
| ENSG00000066230 | ENSG00000188783 | ENSG00000128594 |
| ENSG00000177272 | ENSG00000272911 | ENSG00000112902 |
| ENSG00000198680 | ENSG00000164330 | ENSG00000173535 |
| ENSG00000173114 | ENSG00000181652 | ENSG00000076662 |
| ENSG00000230453 | ENSG00000274902 | ENSG00000280106 |
| ENSG00000235601 | ENSG00000183486 | ENSG00000079257 |
| ENSG00000145362 | ENSG00000259495 | ENSG00000026559 |
| ENSG00000243894 | ENSG00000273113 | ENSG00000269859 |
| ENSG00000115461 | ENSG00000126733 | ENSG00000197959 |
| ENSG00000162975 | ENSG00000274121 | ENSG00000251992 |
| ENSG00000227304 | ENSG00000129521 | ENSG00000273133 |
| ENSG00000135744 | ENSG00000162676 | ENSG00000106571 |
| ENSG00000232599 | ENSG00000144619 | ENSG00000176920 |
| ENSG00000277918 | ENSG00000259592 | ENSG00000242748 |
| ENSG00000227248 | ENSG00000163623 | ENSG00000257696 |
| ENSG00000141293 | ENSG00000081041 | ENSG00000184545 |
| ENSG00000148671 | ENSG00000232139 | ENSG00000211968 |
| ENSG00000137265 | ENSG00000273258 | ENSG00000147509 |
| ENSG00000279068 | ENSG00000174348 | ENSG00000158869 |
| ENSG00000256450 | ENSG00000169297 | ENSG00000189120 |
| ENSG00000084628 | ENSG00000152954 | ENSG00000087076 |
| ENSG00000128713 | ENSG00000075275 | ENSG00000229492 |
| ENSG00000142700 | ENSG00000261395 | ENSG00000180354 |
| ENSG00000136002 | ENSG00000185101 | ENSG00000135299 |
| ENSG00000119698 | ENSG00000168135 | ENSG00000216475 |
| ENSG00000081853 | ENSG00000258844 | ENSG00000244289 |
| ENSG00000064763 | ENSG00000265590 | ENSG00000272789 |
| ENSG00000230699 | ENSG00000266932 | ENSG00000226823 |
| ENSG00000196735 | ENSG00000164398 | ENSG00000273837 |
| ENSG00000102452 | ENSG00000162849 | ENSG00000135547 |
| ENSG00000261040 | ENSG00000156968 | ENSG00000276292 |
| ENSG00000136274 | ENSG00000175556 | ENSG00000073150 |
| ENSG00000232166 | ENSG00000189419 | ENSG00000279642 |
| ENSG00000249242 | ENSG00000130649 | ENSG00000141448 |
| ENSG00000137868 | ENSG00000224626 | ENSG00000174125 |
| ENSG00000251866 | ENSG00000242600 | ENSG00000229054 |
| ENSG00000229184 | ENSG00000264676 | ENSG00000270640 |
| ENSG00000135454 | ENSG00000145491 | ENSG00000104953 |
| ENSG00000103316 | ENSG00000123096 | ENSG00000197859 |
| ENSG00000169071 | ENSG00000238213 | ENSG00000259544 |
| ENSG00000203799 | ENSG00000282980 | ENSG00000140678 |
| ENSG00000234373 | ENSG00000188158 | ENSG00000229797 |
| ENSG00000223247 | ENSG00000256725 | ENSG00000233170 |
| ENSG00000196878 | ENSG00000078098 | ENSG00000101194 |
| ENSG00000262074 | ENSG00000164185 | ENSG00000115884 |
| ENSG00000213483 | ENSG00000047617 | ENSG00000242419 |
| ENSG00000275325 | ENSG00000260011 | ENSG00000244671 |
| ENSG00000163923 | ENSG00000280381 | ENSG00000185130 |
| ENSG00000249509 | ENSG00000137558 | ENSG00000197852 |
| ENSG00000241261 | ENSG00000205502 | ENSG00000232855 |
| ENSG00000135363 | ENSG00000272668 | ENSG00000241832 |
| ENSG00000121075 | ENSG00000226874 | ENSG00000007171 |
| ENSG00000128714 | ENSG00000138772 | ENSG00000269889 |
| ENSG00000178233 | ENSG00000257829 | ENSG00000260511 |
| ENSG00000244405 | ENSG00000086289 | ENSG00000181847 |
| ENSG00000078725 | ENSG00000240183 | ENSG00000182578 |
| ENSG00000143365 | ENSG00000251194 | ENSG00000237026 |
| ENSG00000224981 | ENSG00000229953 | ENSG00000183273 |
| ENSG00000226864 | ENSG00000269425 | ENSG00000112782 |
| ENSG00000185666 | ENSG00000265095 | ENSG00000277775 |
| ENSG00000181195 | ENSG00000121871 | ENSG00000213398 |
| ENSG00000233559 | ENSG00000125848 | ENSG00000077420 |
| ENSG00000164176 | ENSG00000243319 | ENSG00000271208 |
| ENSG00000224014 | ENSG00000273478 | ENSG00000261857 |
| ENSG00000267376 | ENSG00000118473 | ENSG00000268204 |
| ENSG00000168447 | ENSG00000044524 | ENSG00000261168 |
| ENSG00000201348 | ENSG00000091181 | ENSG00000019991 |
| ENSG00000231967 | ENSG00000196569 | ENSG00000003147 |
| ENSG00000183840 | ENSG00000170571 | ENSG00000258978 |
| ENSG00000186732 | ENSG00000205579 | ENSG00000259807 |
| ENSG00000183734 | ENSG00000200597 | ENSG00000265840 |
| ENSG00000248103 | ENSG00000100342 | ENSG00000229497 |
| ENSG00000107736 | ENSG00000272636 | ENSG00000245711 |
| ENSG00000224302 | ENSG00000243649 | ENSG00000213013 |
| ENSG00000196482 | ENSG00000280399 | ENSG00000108852 |
| ENSG00000164746 | ENSG00000140092 | ENSG00000235990 |
| ENSG00000259064 | ENSG00000072201 | ENSG00000105613 |
| ENSG00000176887 | ENSG00000120708 | ENSG00000164619 |
| ENSG00000237945 | ENSG00000151715 | ENSG00000280153 |
| ENSG00000228723 | ENSG00000270141 | ENSG00000221716 |
| ENSG00000163993 | ENSG00000175886 | ENSG00000234167 |
| ENSG00000081842 | ENSG00000171517 | ENSG00000272864 |
| ENSG00000253361 | ENSG00000162878 | ENSG00000069702 |
| ENSG00000145248 | ENSG00000055813 | ENSG00000250482 |
| ENSG00000139211 | ENSG00000267751 | ENSG00000186369 |
| ENSG00000120693 | ENSG00000255874 | ENSG00000261773 |
| ENSG00000144229 | ENSG00000226321 | ENSG00000087303 |
| ENSG00000115828 | ENSG00000175868 | ENSG00000230470 |
| ENSG00000146938 | ENSG00000180644 | ENSG00000279682 |
| ENSG00000174567 | ENSG00000248790 | ENSG00000087116 |
| ENSG00000129991 | ENSG00000279511 | ENSG00000202198 |
| ENSG00000240694 | ENSG00000201758 | ENSG00000259065 |
| ENSG00000253627 | ENSG00000261068 | ENSG00000254983 |
| ENSG00000206073 | ENSG00000266744 | ENSG00000273321 |
| ENSG00000153789 | ENSG00000228400 | ENSG00000250615 |
| ENSG00000104237 | ENSG00000242732 | ENSG00000168421 |
| ENSG00000143882 | ENSG00000136960 | ENSG00000272068 |
| ENSG00000134398 | ENSG00000146521 | ENSG00000053328 |
| ENSG00000153930 | ENSG00000133115 | ENSG00000161835 |
| ENSG00000272472 | ENSG00000169783 | ENSG00000238228 |
| ENSG00000255618 | ENSG00000154027 | ENSG00000106013 |
| ENSG00000171724 | ENSG00000200418 | ENSG00000267265 |
| ENSG00000243916 | ENSG00000168676 | ENSG00000186517 |
| ENSG00000150893 | ENSG00000273160 | ENSG00000071967 |
| ENSG00000152953 | ENSG00000106689 | ENSG00000258472 |
| ENSG00000280359 | ENSG00000263720 | ENSG00000004838 |
| ENSG00000177108 | ENSG00000276107 | ENSG00000277351 |
| ENSG00000145075 | ENSG00000138606 | ENSG00000240032 |
| ENSG00000188931 | ENSG00000132016 | ENSG00000234456 |
| ENSG00000271981 | ENSG00000070190 | ENSG00000235044 |
| ENSG00000247627 | ENSG00000214725 | ENSG00000113263 |
| ENSG00000187510 | ENSG00000007384 | ENSG00000126262 |
| ENSG00000100336 | ENSG00000236358 | ENSG00000131620 |
| ENSG00000073282 | ENSG00000142871 | ENSG00000225498 |
| ENSG00000269125 | ENSG00000244139 | ENSG00000227992 |
| ENSG00000182263 | ENSG00000163431 | ENSG00000057657 |
| ENSG00000120075 | ENSG00000070886 | ENSG00000166949 |
| ENSG00000250942 | ENSG00000267119 | ENSG00000146006 |
| ENSG00000163885 | ENSG00000229758 | ENSG00000188818 |
| ENSG00000166736 | ENSG00000217275 | ENSG00000272275 |
| ENSG00000251468 | ENSG00000018869 | ENSG00000250796 |
| ENSG00000230524 | ENSG00000112294 | ENSG00000204592 |
| ENSG00000128602 | ENSG00000168306 | ENSG00000198429 |
| ENSG00000196990 | ENSG00000276002 | ENSG00000008513 |
| ENSG00000168824 | ENSG00000119535 | ENSG00000162745 |
| ENSG00000236711 | ENSG00000100385 | ENSG00000176124 |
| ENSG00000261478 | ENSG00000090104 | ENSG00000142494 |
| ENSG00000271361 | ENSG00000239948 | ENSG00000198829 |
| ENSG00000154451 | ENSG00000085552 | ENSG00000166033 |
| ENSG00000065675 | ENSG00000171714 | ENSG00000277534 |
| ENSG00000250237 | ENSG00000239595 | ENSG00000116774 |
| ENSG00000178445 | ENSG00000272904 | ENSG00000146013 |
| ENSG00000226912 | ENSG00000144369 | ENSG00000172296 |
| ENSG00000153292 | ENSG00000106333 | ENSG00000127578 |
| ENSG00000201574 | ENSG00000185100 | ENSG00000168348 |
| ENSG00000140470 | ENSG00000122679 | ENSG00000268220 |
| ENSG00000138622 | ENSG00000261065 | ENSG00000253671 |
| ENSG00000258650 | ENSG00000271426 | ENSG00000171551 |
| ENSG00000165061 | ENSG00000152284 | ENSG00000211655 |
| ENSG00000175262 | ENSG00000263893 | ENSG00000175899 |
| ENSG00000065320 | ENSG00000249679 | ENSG00000144893 |
| ENSG00000256070 | ENSG00000149573 | ENSG00000175906 |
| ENSG00000130600 | ENSG00000277223 | ENSG00000156011 |
| ENSG00000221818 | ENSG00000261377 | ENSG00000171450 |
| ENSG00000184160 | ENSG00000229989 | ENSG00000159871 |
| ENSG00000178171 | ENSG00000074317 | ENSG00000231711 |
| ENSG00000101825 | ENSG00000103257 | ENSG00000213967 |
| ENSG00000260711 | ENSG00000206417 | ENSG00000268047 |
| ENSG00000165023 | ENSG00000136514 | ENSG00000184785 |
| ENSG00000213088 | ENSG00000170049 | ENSG00000174669 |
| ENSG00000215241 | ENSG00000164691 | ENSG00000181378 |
| ENSG00000073737 | ENSG00000189221 | ENSG00000168528 |
| ENSG00000105523 | ENSG00000160145 | ENSG00000203648 |
| ENSG00000183798 | ENSG00000278351 | ENSG00000177098 |
| ENSG00000182631 | ENSG00000259969 | ENSG00000275142 |
| ENSG00000137078 | ENSG00000225218 | ENSG00000197629 |
| ENSG00000262869 | ENSG00000273100 | ENSG00000234929 |
| ENSG00000111344 | ENSG00000163435 | ENSG00000253988 |
| ENSG00000280432 | ENSG00000150556 | ENSG00000272123 |
| ENSG00000185652 | ENSG00000204287 | ENSG00000199420 |
| ENSG00000117525 | ENSG00000183833 | ENSG00000106477 |
| ENSG00000138115 | ENSG00000176046 | ENSG00000162572 |
| ENSG00000166501 | ENSG00000188112 | ENSG00000257008 |
| ENSG00000134853 | ENSG00000224769 | ENSG00000228719 |
| ENSG00000185274 | ENSG00000101255 | ENSG00000099960 |
| ENSG00000226454 | ENSG00000176381 | ENSG00000137501 |
| ENSG00000214215 | ENSG00000176659 | ENSG00000152952 |
| ENSG00000271063 | ENSG00000258711 | ENSG00000088340 |
| ENSG00000260600 | ENSG00000259109 | ENSG00000161638 |
| ENSG00000233942 | ENSG00000231699 | ENSG00000120833 |
| ENSG00000176029 | ENSG00000060558 | ENSG00000233885 |
| ENSG00000237838 | ENSG00000282390 | ENSG00000267374 |
| ENSG00000255987 | ENSG00000064270 | ENSG00000249734 |
| ENSG00000177459 | ENSG00000167011 | ENSG00000259336 |
| ENSG00000224307 | ENSG00000079215 | ENSG00000261673 |
| ENSG00000112319 | ENSG00000215068 | ENSG00000088756 |
| ENSG00000064787 | ENSG00000243836 | ENSG00000239300 |
| ENSG00000184515 | ENSG00000204179 | ENSG00000278828 |
| ENSG00000166450 | ENSG00000274290 | ENSG00000197721 |
| ENSG00000136918 | ENSG00000242768 | ENSG00000105131 |
| ENSG00000268889 | ENSG00000228399 | ENSG00000223511 |
| ENSG00000100206 | ENSG00000236924 | ENSG00000272934 |
| ENSG00000173253 | ENSG00000138795 | ENSG00000166147 |
| ENSG00000166159 | ENSG00000060138 | ENSG00000261603 |
| ENSG00000136546 | ENSG00000112530 | ENSG00000150782 |
| ENSG00000101746 | ENSG00000111319 | ENSG00000279360 |
| ENSG00000171649 | ENSG00000226686 | ENSG00000254855 |
| ENSG00000222386 | ENSG00000259780 | ENSG00000261787 |
| ENSG00000133110 | ENSG00000135678 | ENSG00000260400 |
| ENSG00000137965 | ENSG00000170166 | ENSG00000174607 |
| ENSG00000104321 | ENSG00000130584 | ENSG00000130005 |
| ENSG00000232946 | ENSG00000157613 | ENSG00000251609 |
| ENSG00000108691 | ENSG00000166016 | ENSG00000159674 |
| ENSG00000233451 | ENSG00000241155 | ENSG00000243954 |
| ENSG00000136383 | ENSG00000146700 | ENSG00000164116 |
| ENSG00000175267 | ENSG00000220771 | ENSG00000167972 |
| ENSG00000225378 | ENSG00000253305 | ENSG00000258744 |
| ENSG00000167244 | ENSG00000181019 | ENSG00000267597 |
| ENSG00000262576 | ENSG00000276754 | ENSG00000265342 |
| ENSG00000184226 | ENSG00000214401 | ENSG00000167037 |
| ENSG00000158486 | ENSG00000253349 | ENSG00000166278 |
| ENSG00000166596 | ENSG00000252699 | ENSG00000156049 |
| ENSG00000145864 | ENSG00000092607 | ENSG00000266389 |
| ENSG00000169429 | ENSG00000172403 | ENSG00000241985 |
| ENSG00000280439 | ENSG00000237273 | ENSG00000273983 |
| ENSG00000122574 | ENSG00000230461 | ENSG00000144227 |
| ENSG00000259937 | ENSG00000167656 | ENSG00000253755 |
| ENSG00000058404 | ENSG00000280070 | ENSG00000227036 |
| ENSG00000259834 | ENSG00000226465 | ENSG00000207475 |
| ENSG00000171451 | ENSG00000280229 | ENSG00000132026 |
| ENSG00000176601 | ENSG00000197253 | ENSG00000082397 |
| ENSG00000279439 | ENSG00000217236 | ENSG00000255320 |
| ENSG00000171094 | ENSG00000149256 | ENSG00000100292 |
| ENSG00000138759 | ENSG00000201581 | ENSG00000170153 |
| ENSG00000108244 | ENSG00000133106 | ENSG00000272836 |
| ENSG00000278642 | ENSG00000207340 | ENSG00000173156 |
| ENSG00000231795 | ENSG00000227053 | ENSG00000134250 |
| ENSG00000158560 | ENSG00000081237 | ENSG00000162511 |
| ENSG00000148123 | ENSG00000187372 | ENSG00000132793 |
| ENSG00000211772 | ENSG00000230131 | ENSG00000206072 |
| ENSG00000279551 | ENSG00000263755 | ENSG00000277502 |
| ENSG00000169550 | ENSG00000166813 | ENSG00000259071 |
| ENSG00000259641 | ENSG00000226779 | ENSG00000130433 |
| ENSG00000231906 | ENSG00000162739 | ENSG00000221303 |
| ENSG00000264845 | ENSG00000143494 | ENSG00000275995 |
| ENSG00000184343 | ENSG00000280137 | ENSG00000187720 |
| ENSG00000132975 | ENSG00000270808 | ENSG00000235903 |
| ENSG00000267508 | ENSG00000173369 | ENSG00000135094 |
| ENSG00000145708 | ENSG00000248144 | ENSG00000197587 |
| ENSG00000178919 | ENSG00000280089 | ENSG00000254207 |
| ENSG00000263551 | ENSG00000120820 | ENSG00000163823 |
| ENSG00000256229 | ENSG00000184497 | ENSG00000135842 |
| ENSG00000185760 | ENSG00000185585 | ENSG00000266402 |
| ENSG00000222810 | ENSG00000124256 | ENSG00000204264 |
| ENSG00000113805 | ENSG00000163629 | ENSG00000168899 |
| ENSG00000133710 | ENSG00000133640 | ENSG00000102024 |
| ENSG00000235141 | ENSG00000110195 | ENSG00000129595 |
| ENSG00000115363 | ENSG00000243364 | ENSG00000280238 |
| ENSG00000204516 | ENSG00000102554 | ENSG00000279637 |
| ENSG00000260934 | ENSG00000203780 | ENSG00000135899 |
| ENSG00000234695 | ENSG00000172366 | ENSG00000154263 |
| ENSG00000105976 | ENSG00000185055 | ENSG00000234293 |
| ENSG00000066468 | ENSG00000163565 | ENSG00000204388 |
| ENSG00000204442 | ENSG00000252212 | ENSG00000130540 |
| ENSG00000236824 | ENSG00000264176 | ENSG00000231010 |
| ENSG00000166920 | ENSG00000270035 | ENSG00000251073 |
| ENSG00000218336 | ENSG00000268683 | ENSG00000153283 |
| ENSG00000058085 | ENSG00000271105 | ENSG00000108405 |
| ENSG00000117016 | ENSG00000278498 | ENSG00000185499 |
| ENSG00000252087 | ENSG00000240890 | ENSG00000232810 |
| ENSG00000142102 | ENSG00000169507 | ENSG00000271384 |
| ENSG00000224185 | ENSG00000205181 | ENSG00000272555 |
| ENSG00000154310 | ENSG00000164532 | ENSG00000103335 |
| ENSG00000181449 | ENSG00000137561 | ENSG00000264083 |
| ENSG00000267541 | ENSG00000095739 | ENSG00000248449 |
| ENSG00000106772 | ENSG00000162598 | ENSG00000230105 |
| ENSG00000204092 | ENSG00000063015 | ENSG00000254370 |
| ENSG00000276410 | ENSG00000275417 | ENSG00000200959 |
| ENSG00000154162 | ENSG00000177359 | ENSG00000230882 |
| ENSG00000133101 | ENSG00000128274 | ENSG00000198570 |
| ENSG00000177707 | ENSG00000197632 | ENSG00000254590 |
| ENSG00000124491 | ENSG00000101162 | ENSG00000272678 |
| ENSG00000284724 | ENSG00000174740 | ENSG00000244061 |
| ENSG00000272583 | ENSG00000159247 | ENSG00000088899 |
| ENSG00000271715 | ENSG00000227470 | ENSG00000203724 |
| ENSG00000155760 | ENSG00000253379 | ENSG00000256968 |
| ENSG00000128564 | ENSG00000223561 | ENSG00000259941 |
| ENSG00000276434 | ENSG00000179799 | ENSG00000171903 |
| ENSG00000183580 | ENSG00000196353 | ENSG00000239590 |
| ENSG00000252118 | ENSG00000234233 | ENSG00000229267 |
| ENSG00000175544 | ENSG00000219133 | ENSG00000205978 |
| ENSG00000214318 | ENSG00000252854 | ENSG00000154258 |
| ENSG00000270714 | ENSG00000230076 | ENSG00000127951 |
| ENSG00000255434 | ENSG00000225091 | ENSG00000103269 |
| ENSG00000253683 | ENSG00000263707 | ENSG00000121552 |
| ENSG00000189366 | ENSG00000122862 | ENSG00000268154 |
| ENSG00000165140 | ENSG00000257231 | ENSG00000137877 |
| ENSG00000145147 | ENSG00000283982 | ENSG00000224064 |
| ENSG00000144057 | ENSG00000279441 | ENSG00000096696 |
| ENSG00000164076 | ENSG00000280216 | ENSG00000276545 |
| ENSG00000064300 | ENSG00000244245 | ENSG00000181381 |
| ENSG00000271817 | ENSG00000169908 | ENSG00000244879 |
| ENSG00000222414 | ENSG00000006016 | ENSG00000078589 |
| ENSG00000154928 | ENSG00000144583 | ENSG00000279095 |
| ENSG00000222076 | ENSG00000169851 | ENSG00000187513 |
| ENSG00000168685 | ENSG00000267128 | ENSG00000205413 |
| ENSG00000250101 | ENSG00000254522 | ENSG00000147576 |
| ENSG00000169302 | ENSG00000276956 | ENSG00000265683 |
| ENSG00000164764 | ENSG00000175445 | ENSG00000279659 |
| ENSG00000111799 | ENSG00000205111 | ENSG00000231744 |
| ENSG00000125851 | ENSG00000181218 | ENSG00000282961 |
| ENSG00000178828 | ENSG00000187244 | ENSG00000197889 |
| ENSG00000181291 | ENSG00000110092 | ENSG00000162998 |
| ENSG00000180347 | ENSG00000012779 | ENSG00000186314 |
| ENSG00000162631 | ENSG00000224020 | ENSG00000008441 |
| ENSG00000185565 | ENSG00000167100 | ENSG00000266094 |
| ENSG00000232558 | ENSG00000269867 | ENSG00000226216 |
| ENSG00000106538 | ENSG00000073756 | ENSG00000217404 |
| ENSG00000164379 | ENSG00000240098 | ENSG00000173404 |
| ENSG00000079841 | ENSG00000241278 | ENSG00000142910 |
| ENSG00000162931 | ENSG00000269899 | ENSG00000278893 |
| ENSG00000231662 | ENSG00000080200 | ENSG00000235162 |
| ENSG00000089225 | ENSG00000233073 | ENSG00000233806 |
| ENSG00000111052 | ENSG00000125740 | ENSG00000283913 |
| ENSG00000241416 | ENSG00000258692 | ENSG00000243702 |
| ENSG00000249992 | ENSG00000196917 | ENSG00000148175 |
| ENSG00000278318 | ENSG00000165548 | ENSG00000237686 |
| ENSG00000101938 | ENSG00000152784 | ENSG00000275345 |
| ENSG00000143196 | ENSG00000267459 | ENSG00000231765 |
| ENSG00000255487 | ENSG00000254556 | ENSG00000070182 |
| ENSG00000188162 | ENSG00000213190 | ENSG00000260014 |
| ENSG00000232445 | ENSG00000250486 | ENSG00000260615 |
| ENSG00000149295 | ENSG00000105255 | ENSG00000227051 |
| ENSG00000266282 | ENSG00000178235 | ENSG00000260586 |
| ENSG00000222627 | ENSG00000279599 | ENSG00000026025 |
| ENSG00000134873 | ENSG00000260139 | ENSG00000146859 |
| ENSG00000201801 | ENSG00000220868 | ENSG00000273669 |
| ENSG00000106178 | ENSG00000146242 | ENSG00000233719 |
| ENSG00000125398 | ENSG00000234602 | ENSG00000244230 |
| ENSG00000198963 | ENSG00000080573 | ENSG00000229324 |
| ENSG00000155761 | ENSG00000269514 | ENSG00000173406 |
| ENSG00000248540 | ENSG00000158815 | ENSG00000255909 |
| ENSG00000163710 | ENSG00000189280 | ENSG00000262165 |
| ENSG00000280002 | ENSG00000156206 | ENSG00000146072 |
| ENSG00000157856 | ENSG00000215217 | ENSG00000268707 |
| ENSG00000172156 | ENSG00000223198 | ENSG00000261371 |
| ENSG00000144810 | ENSG00000240173 | ENSG00000064666 |
| ENSG00000131771 | ENSG00000110881 | ENSG00000105464 |
| ENSG00000278052 | ENSG00000244018 | ENSG00000115738 |
| ENSG00000262890 | ENSG00000165730 | ENSG00000273001 |
| ENSG00000134121 | ENSG00000006468 | ENSG00000229352 |
| ENSG00000144857 | ENSG00000243723 | ENSG00000273687 |
| ENSG00000231453 | ENSG00000274367 | ENSG00000248785 |
| ENSG00000156564 | ENSG00000170775 | ENSG00000202252 |
| ENSG00000198580 | ENSG00000258412 | ENSG00000184611 |
| ENSG00000258215 | ENSG00000224530 | ENSG00000116396 |
| ENSG00000235389 | ENSG00000278886 | ENSG00000248753 |
| ENSG00000188404 | ENSG00000212163 | ENSG00000234883 |
| ENSG00000261488 | ENSG00000244618 | ENSG00000279192 |
| ENSG00000266265 | ENSG00000244256 | ENSG00000128645 |
| ENSG00000169248 | ENSG00000230630 | ENSG00000188613 |
| ENSG00000254786 | ENSG00000237187 | ENSG00000110934 |
| ENSG00000140563 | ENSG00000060982 | ENSG00000213598 |
| ENSG00000281406 | ENSG00000258884 | ENSG00000272338 |
| ENSG00000217331 | ENSG00000260209 | ENSG00000235760 |
| ENSG00000275155 | ENSG00000127252 | ENSG00000249669 |
| ENSG00000101187 | ENSG00000185404 | ENSG00000185532 |
| ENSG00000152672 | ENSG00000270773 | ENSG00000187999 |
| ENSG00000178568 | ENSG00000174576 | ENSG00000164287 |
| ENSG00000188064 | ENSG00000256751 | ENSG00000197646 |
| ENSG00000133878 | ENSG00000213754 | ENSG00000265791 |
| ENSG00000164946 | ENSG00000165912 | ENSG00000221963 |
| ENSG00000259450 | ENSG00000112812 | ENSG00000274001 |
| ENSG00000156738 | ENSG00000103184 | ENSG00000136378 |
| ENSG00000162188 | ENSG00000266709 | ENSG00000182013 |
| ENSG00000253208 | ENSG00000196132 | ENSG00000171388 |
| ENSG00000274225 | ENSG00000143119 | ENSG00000107593 |
| ENSG00000088836 | ENSG00000198934 | ENSG00000168874 |
| ENSG00000238250 | ENSG00000164647 | ENSG00000175315 |
| ENSG00000162692 | ENSG00000224349 | ENSG00000172123 |
| ENSG00000279932 | ENSG00000197106 | ENSG00000244623 |
| ENSG00000113763 | ENSG00000131196 | ENSG00000248485 |
| ENSG00000273325 | ENSG00000136997 | ENSG00000260366 |
| ENSG00000226777 | ENSG00000158816 | ENSG00000154096 |
| ENSG00000124215 | ENSG00000239794 | ENSG00000274184 |
| ENSG00000130701 | ENSG00000074416 | ENSG00000283098 |
| ENSG00000279702 | ENSG00000277235 | ENSG00000251164 |
| ENSG00000213886 | ENSG00000153714 | ENSG00000164309 |
| ENSG00000248587 | ENSG00000078081 | ENSG00000255143 |
| ENSG00000110076 | ENSG00000213085 | ENSG00000184371 |
| ENSG00000268912 | ENSG00000089127 | ENSG00000133962 |
| ENSG00000115844 | ENSG00000254271 | ENSG00000137872 |
| ENSG00000127084 | ENSG00000278746 | ENSG00000197576 |
| ENSG00000275538 | ENSG00000118307 | ENSG00000090006 |
| ENSG00000207112 | ENSG00000167600 | ENSG00000120645 |
| ENSG00000259056 | ENSG00000231713 | ENSG00000213669 |
| ENSG00000165995 | ENSG00000240966 | ENSG00000140479 |
| ENSG00000185352 | ENSG00000145335 | ENSG00000187260 |
| ENSG00000129757 | ENSG00000138646 | ENSG00000269743 |
| ENSG00000196358 | ENSG00000121005 | ENSG00000203747 |
| ENSG00000230391 | ENSG00000137142 | ENSG00000255428 |
| ENSG00000159167 | ENSG00000134757 | ENSG00000243562 |
| ENSG00000162069 | ENSG00000186479 | ENSG00000171044 |
| ENSG00000136689 | ENSG00000271420 | ENSG00000284681 |
| ENSG00000260265 | ENSG00000271109 | ENSG00000142959 |
| ENSG00000124302 | ENSG00000157005 | ENSG00000278873 |
| ENSG00000168621 | ENSG00000233540 | ENSG00000168490 |
| ENSG00000162004 | ENSG00000208308 | ENSG00000267127 |
| ENSG00000117477 | ENSG00000146904 | ENSG00000123700 |
| ENSG00000064205 | ENSG00000145287 | ENSG00000278035 |
| ENSG00000129990 | ENSG00000174498 | ENSG00000243710 |
| ENSG00000079931 | ENSG00000123009 | ENSG00000260310 |
| ENSG00000200895 | ENSG00000260838 | ENSG00000205930 |
| ENSG00000007062 | ENSG00000115009 | ENSG00000133328 |
| ENSG00000175745 | ENSG00000015592 | ENSG00000241697 |
| ENSG00000100626 | ENSG00000111837 | ENSG00000270127 |
| ENSG00000256982 | ENSG00000260089 | ENSG00000185386 |
| ENSG00000070601 | ENSG00000280604 | ENSG00000198929 |
| ENSG00000233080 | ENSG00000176597 | ENSG00000256262 |
| ENSG00000258675 | ENSG00000245532 | ENSG00000137726 |
| ENSG00000109819 | ENSG00000260740 | ENSG00000274294 |
| ENSG00000198848 | ENSG00000250510 | ENSG00000232894 |
| ENSG00000261212 | ENSG00000212371 | ENSG00000164484 |
| ENSG00000233930 | ENSG00000005844 | ENSG00000174600 |
| ENSG00000136842 | ENSG00000258111 | ENSG00000269054 |
| ENSG00000273816 | ENSG00000273164 | ENSG00000162669 |
| ENSG00000144681 | ENSG00000240457 | ENSG00000187134 |
| ENSG00000240480 | ENSG00000172116 | ENSG00000169604 |
| ENSG00000237152 | ENSG00000227021 | ENSG00000271379 |
| ENSG00000177398 | ENSG00000142089 | ENSG00000273156 |
| ENSG00000250208 | ENSG00000238123 | ENSG00000141665 |
| ENSG00000111058 | ENSG00000224041 | ENSG00000240429 |
| ENSG00000116299 | ENSG00000132965 | ENSG00000115896 |
| ENSG00000253602 | ENSG00000149131 | ENSG00000147872 |
| ENSG00000087245 | ENSG00000239627 | ENSG00000046889 |
| ENSG00000269392 | ENSG00000254936 | ENSG00000255343 |
| ENSG00000107242 | ENSG00000116729 | ENSG00000278492 |
| ENSG00000182866 | ENSG00000183346 | ENSG00000166148 |
| ENSG00000246627 | ENSG00000255270 | ENSG00000135747 |
| ENSG00000230873 | ENSG00000186009 | ENSG00000234498 |
| ENSG00000163661 | ENSG00000163995 | ENSG00000087250 |
| ENSG00000262655 | ENSG00000173040 | ENSG00000254330 |
| ENSG00000213519 | ENSG00000173372 | ENSG00000111186 |
| ENSG00000132554 | ENSG00000111907 | ENSG00000105997 |
| ENSG00000168658 | ENSG00000140950 | ENSG00000264714 |
| ENSG00000144278 | ENSG00000091592 | ENSG00000099194 |
| ENSG00000162782 | ENSG00000165626 | ENSG00000172673 |
| ENSG00000280453 | ENSG00000237989 | ENSG00000254689 |
| ENSG00000277950 | ENSG00000163273 | ENSG00000254838 |
| ENSG00000123453 | ENSG00000100060 | ENSG00000232736 |
| ENSG00000115457 | ENSG00000160791 | ENSG00000147606 |
| ENSG00000079385 | ENSG00000221866 | ENSG00000130768 |
| ENSG00000100739 | ENSG00000038427 | ENSG00000103056 |
| ENSG00000278099 | ENSG00000232006 | ENSG00000165238 |
| ENSG00000152128 | ENSG00000145416 | ENSG00000266378 |
| ENSG00000183873 | ENSG00000179546 | ENSG00000267769 |
| ENSG00000176769 | ENSG00000279981 | ENSG00000260874 |
| ENSG00000178773 | ENSG00000237529 | ENSG00000171004 |
| ENSG00000184305 | ENSG00000217643 | ENSG00000251580 |
| ENSG00000186493 | ENSG00000206384 | ENSG00000227704 |
| ENSG00000006283 | ENSG00000164972 | ENSG00000104267 |
| ENSG00000278840 | ENSG00000136999 | ENSG00000272861 |
| ENSG00000134548 | ENSG00000251733 | ENSG00000227293 |
| ENSG00000230190 | ENSG00000084636 | ENSG00000224029 |
| ENSG00000273712 | ENSG00000248994 | ENSG00000160539 |
| ENSG00000157168 | ENSG00000214290 | ENSG00000262528 |
| ENSG00000034239 | ENSG00000100368 | ENSG00000168913 |
| ENSG00000179913 | ENSG00000145569 | ENSG00000106714 |
| ENSG00000056998 | ENSG00000255020 | ENSG00000235065 |
| ENSG00000258131 | ENSG00000199319 | ENSG00000176720 |
| ENSG00000239942 | ENSG00000263120 | ENSG00000159307 |
| ENSG00000155659 | ENSG00000141639 | ENSG00000134716 |
| ENSG00000151948 | ENSG00000183807 | ENSG00000231544 |
| ENSG00000146038 | ENSG00000272746 | ENSG00000066294 |
| ENSG00000222515 | ENSG00000272161 | ENSG00000272024 |
| ENSG00000130035 | ENSG00000228280 | ENSG00000152076 |
| ENSG00000142609 | ENSG00000254974 | ENSG00000146409 |
| ENSG00000158220 | ENSG00000140853 | ENSG00000274602 |
| ENSG00000253124 | ENSG00000240014 | ENSG00000279693 |
| ENSG00000278847 | ENSG00000188659 | ENSG00000186603 |
| ENSG00000070731 | ENSG00000126353 | ENSG00000272137 |
| ENSG00000283646 | ENSG00000214919 | ENSG00000263050 |
| ENSG00000270332 | ENSG00000152583 | ENSG00000123146 |
| ENSG00000225156 | ENSG00000120738 | ENSG00000101222 |
| ENSG00000197614 | ENSG00000164362 | ENSG00000239521 |
| ENSG00000163618 | ENSG00000261434 | ENSG00000264940 |
| ENSG00000182636 | ENSG00000078549 | ENSG00000183032 |
| ENSG00000258667 | ENSG00000253641 | ENSG00000272354 |
| ENSG00000243915 | ENSG00000271141 | ENSG00000185291 |
| ENSG00000112218 | ENSG00000157734 | ENSG00000278389 |
| ENSG00000277734 | ENSG00000168032 | ENSG00000170542 |
| ENSG00000185046 | ENSG00000227374 | ENSG00000261624 |
| ENSG00000253187 | ENSG00000254678 | ENSG00000204003 |
| ENSG00000236404 | ENSG00000255733 | ENSG00000068079 |
| ENSG00000246082 | ENSG00000104689 | ENSG00000197461 |
| ENSG00000232044 | ENSG00000108700 | ENSG00000261159 |
| ENSG00000249930 | ENSG00000127129 | ENSG00000238015 |
| ENSG00000242808 | ENSG00000278716 | ENSG00000244119 |
| ENSG00000081277 | ENSG00000233216 | ENSG00000100351 |
| ENSG00000189212 | ENSG00000253647 | ENSG00000118492 |
| ENSG00000273032 | ENSG00000267532 | ENSG00000114019 |
| ENSG00000176293 | ENSG00000272183 | ENSG00000265939 |
| ENSG00000261762 | ENSG00000267191 | ENSG00000171126 |
| ENSG00000041982 | ENSG00000254003 | ENSG00000272418 |
| ENSG00000215218 | ENSG00000183586 | ENSG00000163131 |
| ENSG00000273108 | ENSG00000156127 | ENSG00000283480 |
| ENSG00000252757 | ENSG00000112414 | ENSG00000239249 |
| ENSG00000005001 | ENSG00000275162 | ENSG00000164675 |
| ENSG00000230734 | ENSG00000005108 | ENSG00000138741 |
| ENSG00000235518 | ENSG00000275910 | ENSG00000172986 |
| ENSG00000170959 | ENSG00000199436 | ENSG00000173546 |
| ENSG00000234965 | ENSG00000253696 | ENSG00000115641 |
| ENSG00000275805 | ENSG00000167191 | ENSG00000283209 |
| ENSG00000226476 | ENSG00000185774 | ENSG00000272087 |
| ENSG00000228261 | ENSG00000279913 | ENSG00000170011 |
| ENSG00000169442 | ENSG00000106123 | ENSG00000053438 |
| ENSG00000169122 | ENSG00000170745 | ENSG00000154188 |
| ENSG00000274883 | ENSG00000188263 | ENSG00000261211 |
| ENSG00000113211 | ENSG00000100625 | ENSG00000279834 |
| ENSG00000143297 | ENSG00000282851 | ENSG00000005981 |
| ENSG00000258788 | ENSG00000088386 | ENSG00000184261 |
| ENSG00000279698 | ENSG00000228594 | ENSG00000223928 |
| ENSG00000229278 | ENSG00000184113 | ENSG00000086991 |
| ENSG00000063127 | ENSG00000188820 | ENSG00000276768 |
| ENSG00000153721 | ENSG00000267260 | ENSG00000090659 |
| ENSG00000198759 | ENSG00000141968 | ENSG00000164035 |
| ENSG00000228798 | ENSG00000166342 | ENSG00000276772 |
| ENSG00000163687 | ENSG00000279692 | ENSG00000198074 |
| ENSG00000167646 | ENSG00000129946 | ENSG00000228485 |
| ENSG00000253020 | ENSG00000101443 | ENSG00000280169 |
| ENSG00000173702 | ENSG00000277159 | ENSG00000205838 |
| ENSG00000206432 | ENSG00000101445 | ENSG00000271011 |
| ENSG00000230397 | ENSG00000130635 | ENSG00000141506 |
| ENSG00000089847 | ENSG00000125845 | ENSG00000122863 |
| ENSG00000215386 | ENSG00000130653 | ENSG00000228036 |
| ENSG00000250098 | ENSG00000255231 | ENSG00000196628 |
| ENSG00000175749 | ENSG00000198417 | ENSG00000243738 |
| ENSG00000111885 | ENSG00000151023 | ENSG00000181234 |
| ENSG00000228914 | ENSG00000099822 | ENSG00000119771 |
| ENSG00000198535 | ENSG00000100453 | ENSG00000250490 |
| ENSG00000104490 | ENSG00000135116 | ENSG00000259167 |
| ENSG00000151640 | ENSG00000143816 | ENSG00000019549 |
| ENSG00000211658 | ENSG00000258513 | ENSG00000164749 |
| ENSG00000173930 | ENSG00000267651 | ENSG00000133135 |
| ENSG00000198910 | ENSG00000281333 | ENSG00000206702 |
| ENSG00000137691 | ENSG00000090339 | ENSG00000011600 |
| ENSG00000162543 | ENSG00000091513 | ENSG00000242272 |
| ENSG00000197826 | ENSG00000169174 | ENSG00000188573 |
| ENSG00000225742 | ENSG00000188211 | ENSG00000072952 |
| ENSG00000253006 | ENSG00000180096 | ENSG00000145194 |
| ENSG00000157152 | ENSG00000248859 | ENSG00000174099 |
| ENSG00000139193 | ENSG00000199568 | ENSG00000100065 |
| ENSG00000131849 | ENSG00000268412 | ENSG00000009790 |
| ENSG00000161405 | ENSG00000271172 | ENSG00000105851 |
| ENSG00000272468 | ENSG00000254048 | ENSG00000229422 |
| ENSG00000270207 | ENSG00000154175 | ENSG00000233875 |
| ENSG00000185615 | ENSG00000233237 | ENSG00000272425 |
| ENSG00000116106 | ENSG00000177191 | ENSG00000132688 |
| ENSG00000228412 | ENSG00000109861 | ENSG00000112378 |
| ENSG00000107954 | ENSG00000269933 | ENSG00000206885 |
| ENSG00000174599 | ENSG00000270154 | ENSG00000251141 |
| ENSG00000138735 | ENSG00000154721 | ENSG00000085265 |
| ENSG00000119866 | ENSG00000155816 | ENSG00000260035 |
| ENSG00000144476 | ENSG00000173557 | ENSG00000265745 |
| ENSG00000271503 | ENSG00000138080 | ENSG00000182175 |
| ENSG00000196844 | ENSG00000237436 | ENSG00000165795 |
| ENSG00000147724 | ENSG00000155980 | ENSG00000183943 |
| ENSG00000214776 | ENSG00000275263 | ENSG00000230289 |
| ENSG00000115107 | ENSG00000221164 | ENSG00000139364 |
| ENSG00000281091 | ENSG00000182463 | ENSG00000233311 |
| ENSG00000248350 | ENSG00000163563 | ENSG00000180773 |
| ENSG00000270393 | ENSG00000121297 | ENSG00000141519 |
| ENSG00000204128 | ENSG00000078114 | ENSG00000177990 |
| ENSG00000135144 | ENSG00000277559 | ENSG00000258658 |
| ENSG00000267521 | ENSG00000128340 | ENSG00000251463 |
| ENSG00000139445 | ENSG00000173175 | ENSG00000157227 |
| ENSG00000253910 | ENSG00000201901 | ENSG00000173210 |
| ENSG00000253563 | ENSG00000231521 | ENSG00000102755 |
| ENSG00000224239 | ENSG00000196159 | ENSG00000182162 |
| ENSG00000270093 | ENSG00000112343 | ENSG00000262519 |
| ENSG00000171643 | ENSG00000256721 | ENSG00000227885 |
| ENSG00000166368 | ENSG00000237380 | ENSG00000234915 |
| ENSG00000128482 | ENSG00000100767 | ENSG00000140403 |
| ENSG00000251532 | ENSG00000139629 | ENSG00000143226 |
| ENSG00000006071 | ENSG00000214391 | ENSG00000242657 |
| ENSG00000153347 | ENSG00000113361 | ENSG00000232387 |
| ENSG00000206199 | ENSG00000197168 | ENSG00000074966 |
| ENSG00000186469 | ENSG00000115380 | ENSG00000136404 |
| ENSG00000119283 | ENSG00000233251 | ENSG00000105426 |
| ENSG00000240031 | ENSG00000256211 | ENSG00000248925 |
| ENSG00000118849 | ENSG00000188643 | ENSG00000131831 |
| ENSG00000166923 | ENSG00000131044 | ENSG00000123165 |
| ENSG00000198729 | ENSG00000125257 | ENSG00000283156 |
| ENSG00000244004 | ENSG00000165325 | ENSG00000179292 |
| ENSG00000230398 | ENSG00000272922 | ENSG00000013725 |
| ENSG00000141934 | ENSG00000179178 | ENSG00000243445 |
| ENSG00000244632 | ENSG00000231233 | ENSG00000155926 |
| ENSG00000130208 | ENSG00000281641 | ENSG00000257674 |
| ENSG00000234626 | ENSG00000174808 | ENSG00000230445 |
| ENSG00000183508 | ENSG00000198774 | ENSG00000211445 |
| ENSG00000114251 | ENSG00000163082 | ENSG00000233304 |
| ENSG00000206579 | ENSG00000272170 | ENSG00000144730 |
| ENSG00000176490 | ENSG00000237903 | ENSG00000162390 |
| ENSG00000114529 | ENSG00000203734 | ENSG00000237200 |
| ENSG00000134323 | ENSG00000010278 | ENSG00000256011 |
| ENSG00000112276 | ENSG00000272851 | ENSG00000159231 |
| ENSG00000172478 | ENSG00000168546 | ENSG00000232097 |
| ENSG00000270492 | ENSG00000146648 | ENSG00000163430 |
| ENSG00000259017 | ENSG00000272564 | ENSG00000213711 |
| ENSG00000246223 | ENSG00000225026 | ENSG00000232526 |
| ENSG00000166432 | ENSG00000259460 | ENSG00000259138 |
| ENSG00000173376 | ENSG00000103569 | ENSG00000213057 |
| ENSG00000168542 | ENSG00000255595 | ENSG00000107249 |
| ENSG00000104783 | ENSG00000256870 | ENSG00000243675 |
| ENSG00000231612 | ENSG00000007908 | ENSG00000003436 |
| ENSG00000168843 | ENSG00000085831 | ENSG00000259088 |
| ENSG00000153707 | ENSG00000236735 | ENSG00000270178 |
| ENSG00000280119 | ENSG00000145730 | ENSG00000177932 |
| ENSG00000225681 | ENSG00000231515 | ENSG00000278668 |
| ENSG00000050555 | ENSG00000256720 | ENSG00000163884 |
| ENSG00000162654 | ENSG00000256420 | ENSG00000278740 |
| ENSG00000179902 | ENSG00000179774 | ENSG00000182118 |
| ENSG00000267327 | ENSG00000280073 | ENSG00000272940 |
| ENSG00000049540 | ENSG00000233695 | ENSG00000110318 |
| ENSG00000275367 | ENSG00000178752 | ENSG00000121594 |
| ENSG00000243650 | ENSG00000143502 | ENSG00000253438 |
| ENSG00000196074 | ENSG00000006740 | ENSG00000156966 |
| ENSG00000243680 | ENSG00000178947 | ENSG00000079263 |
| ENSG00000108176 | ENSG00000121440 | ENSG00000226928 |
| ENSG00000196368 | ENSG00000241156 | ENSG00000232437 |
| ENSG00000102362 | ENSG00000223772 | ENSG00000198598 |
| ENSG00000241756 | ENSG00000155974 | ENSG00000278527 |
| ENSG00000277118 | ENSG00000258300 | ENSG00000110042 |
| ENSG00000206120 | ENSG00000270765 | ENSG00000251476 |
| ENSG00000256574 | ENSG00000155066 | ENSG00000201302 |
| ENSG00000234692 | ENSG00000163631 | ENSG00000118194 |
| ENSG00000229512 | ENSG00000272721 | ENSG00000239953 |
| ENSG00000234423 | ENSG00000253726 | ENSG00000196660 |
| ENSG00000198217 | ENSG00000252139 | ENSG00000232732 |
| ENSG00000232906 | ENSG00000173627 | ENSG00000154330 |
| ENSG00000185885 | ENSG00000144407 | ENSG00000073711 |
| ENSG00000172260 | ENSG00000199266 | ENSG00000141469 |
| ENSG00000284526 | ENSG00000197134 | ENSG00000134533 |
| ENSG00000094963 | ENSG00000226089 | ENSG00000180152 |
| ENSG00000268333 | ENSG00000229122 | ENSG00000214244 |
| ENSG00000261754 | ENSG00000140297 | ENSG00000277911 |
| ENSG00000254528 | ENSG00000138166 | ENSG00000179104 |
| ENSG00000215146 | ENSG00000134321 | ENSG00000202344 |
| ENSG00000242607 | ENSG00000228222 | ENSG00000163053 |
| ENSG00000118160 | ENSG00000225203 | ENSG00000185989 |
| ENSG00000253767 | ENSG00000235834 | ENSG00000268049 |
| ENSG00000231535 | ENSG00000174950 | ENSG00000283486 |
| ENSG00000141655 | ENSG00000226520 | ENSG00000061455 |
| ENSG00000112541 | ENSG00000253092 | ENSG00000240972 |
| ENSG00000230648 | ENSG00000173826 | ENSG00000163864 |
| ENSG00000226340 | ENSG00000178821 | ENSG00000241529 |
| ENSG00000237361 | ENSG00000130300 | ENSG00000253586 |
| ENSG00000280443 | ENSG00000153551 | ENSG00000199883 |
| ENSG00000151012 | ENSG00000117643 | ENSG00000228741 |
| ENSG00000242795 | ENSG00000254944 | ENSG00000259561 |
| ENSG00000226604 | ENSG00000253666 | ENSG00000264017 |
| ENSG00000217159 | ENSG00000201675 | ENSG00000224189 |
| ENSG00000150471 | ENSG00000126878 | ENSG00000129451 |
| ENSG00000229236 | ENSG00000234383 | ENSG00000236240 |
| ENSG00000176049 | ENSG00000006756 | ENSG00000110324 |
| ENSG00000267034 | ENSG00000055732 | ENSG00000100285 |
| ENSG00000169126 | ENSG00000007237 | ENSG00000119950 |
| ENSG00000146197 | ENSG00000123689 | ENSG00000131981 |
| ENSG00000279067 | ENSG00000109107 | ENSG00000249574 |
| ENSG00000196361 | ENSG00000231864 | ENSG00000272343 |
| ENSG00000102678 | ENSG00000188747 | ENSG00000241809 |
| ENSG00000069188 | ENSG00000265327 | ENSG00000277396 |
| ENSG00000200084 | ENSG00000151790 | ENSG00000129993 |
| ENSG00000243910 | ENSG00000164638 | ENSG00000100450 |
| ENSG00000226562 | ENSG00000270171 | ENSG00000169692 |
| ENSG00000078053 | ENSG00000158445 | ENSG00000105996 |
| ENSG00000234592 | ENSG00000204525 | ENSG00000264294 |
| ENSG00000184905 | ENSG00000251493 | ENSG00000099139 |
| ENSG00000110002 | ENSG00000138675 | ENSG00000164023 |
| ENSG00000144488 | ENSG00000230194 | ENSG00000169896 |
| ENSG00000282381 | ENSG00000171940 | ENSG00000168280 |
| ENSG00000187492 | ENSG00000114270 | ENSG00000247774 |
| ENSG00000220517 | ENSG00000254325 | ENSG00000173559 |
| ENSG00000227850 | ENSG00000203805 | ENSG00000104368 |
| ENSG00000279039 | ENSG00000227857 | ENSG00000279463 |
| ENSG00000235174 | ENSG00000016402 | ENSG00000250148 |
| ENSG00000205488 | ENSG00000103021 | ENSG00000150540 |
| ENSG00000126010 | ENSG00000123560 | ENSG00000272995 |
| ENSG00000252712 | ENSG00000262096 | ENSG00000265185 |
| ENSG00000170369 | ENSG00000230898 | ENSG00000236739 |
| ENSG00000140254 | ENSG00000146215 | ENSG00000185607 |
| ENSG00000224957 | ENSG00000240767 | ENSG00000206811 |
| ENSG00000256481 | ENSG00000277010 | ENSG00000239887 |
| ENSG00000185008 | ENSG00000182667 | ENSG00000112559 |
| ENSG00000158089 | ENSG00000137273 | ENSG00000183644 |
| ENSG00000156689 | ENSG00000103528 | ENSG00000253075 |
| ENSG00000105664 | ENSG00000186642 | ENSG00000270885 |
| ENSG00000228092 | ENSG00000270577 | ENSG00000255256 |
| ENSG00000231227 | ENSG00000253426 | ENSG00000267121 |
| ENSG00000197249 | ENSG00000267056 | ENSG00000186862 |
| ENSG00000102109 | ENSG00000176387 | ENSG00000183578 |
| ENSG00000221750 | ENSG00000151229 | ENSG00000182621 |
| ENSG00000139835 | ENSG00000198133 | ENSG00000270917 |
| ENSG00000081818 | ENSG00000261823 | ENSG00000246465 |
| ENSG00000162433 | ENSG00000258592 | ENSG00000243479 |
| ENSG00000226659 | ENSG00000277829 | ENSG00000280767 |
| ENSG00000212195 | ENSG00000280122 | ENSG00000232368 |
| ENSG00000280431 | ENSG00000183570 | ENSG00000272927 |
| ENSG00000259037 | ENSG00000214067 | ENSG00000214745 |
| ENSG00000172828 | ENSG00000186973 | ENSG00000201315 |
| ENSG00000239919 | ENSG00000162645 | ENSG00000164197 |
| ENSG00000137033 | ENSG00000243398 | ENSG00000233725 |
| ENSG00000153822 | ENSG00000222032 | ENSG00000260027 |
| ENSG00000260337 | ENSG00000225630 | ENSG00000241535 |
| ENSG00000271347 | ENSG00000275126 | ENSG00000275119 |
| ENSG00000134817 | ENSG00000114923 | ENSG00000270850 |
| ENSG00000229500 | ENSG00000134376 | ENSG00000134874 |
| ENSG00000140279 | ENSG00000239224 | ENSG00000227203 |
| ENSG00000101883 | ENSG00000233058 | ENSG00000261342 |
| ENSG00000143858 | ENSG00000275479 | ENSG00000214535 |
| ENSG00000254035 | ENSG00000283122 | ENSG00000187140 |
| ENSG00000100505 | ENSG00000237953 | ENSG00000147251 |
| ENSG00000127325 | ENSG00000283773 | ENSG00000261888 |
| ENSG00000125731 | ENSG00000103489 | ENSG00000178860 |
| ENSG00000239218 | ENSG00000152315 | ENSG00000183682 |
| ENSG00000060656 | ENSG00000269635 | ENSG00000200534 |
| ENSG00000088881 | ENSG00000177283 | ENSG00000274341 |
| ENSG00000249859 | ENSG00000104894 | ENSG00000213608 |
| ENSG00000225138 | ENSG00000275091 | ENSG00000275223 |
| ENSG00000242229 | ENSG00000254895 | ENSG00000157978 |
| ENSG00000170703 | ENSG00000257181 | ENSG00000276097 |
| ENSG00000226642 | ENSG00000130948 | ENSG00000278978 |
| ENSG00000189431 | ENSG00000275175 | ENSG00000136573 |
| ENSG00000238363 | ENSG00000239881 | ENSG00000267254 |
| ENSG00000279075 | ENSG00000160678 | ENSG00000148053 |
| ENSG00000226970 | ENSG00000180447 | ENSG00000227214 |
| ENSG00000164488 | ENSG00000250448 | ENSG00000178562 |
| ENSG00000211652 | ENSG00000242861 | ENSG00000250608 |
| ENSG00000107742 | ENSG00000123610 | ENSG00000017427 |
| ENSG00000200259 | ENSG00000205403 | ENSG00000267390 |
| ENSG00000224965 | ENSG00000240294 | ENSG00000235082 |
| ENSG00000165929 | ENSG00000250986 | ENSG00000279117 |
| ENSG00000144339 | ENSG00000184349 | ENSG00000166558 |
| ENSG00000255035 | ENSG00000196189 | ENSG00000136250 |
| ENSG00000248228 | ENSG00000215771 | ENSG00000234005 |
| ENSG00000186529 | ENSG00000213809 | ENSG00000251648 |
| ENSG00000167107 | ENSG00000277795 | ENSG00000148082 |
| ENSG00000186340 | ENSG00000141574 | ENSG00000259363 |
| ENSG00000229119 | ENSG00000237249 | ENSG00000241002 |
| ENSG00000171385 | ENSG00000249142 | ENSG00000266185 |
| ENSG00000185567 | ENSG00000213420 | ENSG00000121361 |
| ENSG00000166573 | ENSG00000250037 | ENSG00000279400 |
| ENSG00000074370 | ENSG00000144331 | ENSG00000262772 |
| ENSG00000198523 | ENSG00000224221 | ENSG00000259690 |
| ENSG00000118596 | ENSG00000187824 | ENSG00000148841 |
| ENSG00000255622 | ENSG00000260423 | ENSG00000135407 |
| ENSG00000271401 | ENSG00000148848 | ENSG00000261635 |
| ENSG00000152377 | ENSG00000112837 | ENSG00000266503 |
| ENSG00000220988 | ENSG00000101335 | ENSG00000256902 |
| ENSG00000241532 | ENSG00000270607 | ENSG00000232065 |
| ENSG00000114200 | ENSG00000177989 | ENSG00000143797 |
| ENSG00000251131 | ENSG00000276070 | ENSG00000230067 |
| ENSG00000214110 | ENSG00000198944 | ENSG00000089327 |
| ENSG00000204789 | ENSG00000185156 | ENSG00000279497 |
| ENSG00000248015 | ENSG00000138642 | ENSG00000172031 |
| ENSG00000270069 | ENSG00000274210 | ENSG00000248774 |
| ENSG00000282989 | ENSG00000256039 | ENSG00000091136 |
| ENSG00000262714 | ENSG00000102265 | ENSG00000164440 |
| ENSG00000101463 | ENSG00000090776 | ENSG00000021300 |
| ENSG00000203995 | ENSG00000184363 | ENSG00000226124 |
| ENSG00000153093 | ENSG00000237731 | ENSG00000226158 |
| ENSG00000230828 | ENSG00000184350 | ENSG00000196739 |
| ENSG00000163071 | ENSG00000205622 | ENSG00000187123 |
| ENSG00000254010 | ENSG00000205213 | ENSG00000262877 |
| ENSG00000160471 | ENSG00000226965 | ENSG00000174672 |
| ENSG00000188817 | ENSG00000267547 | ENSG00000105974 |
| ENSG00000224273 | ENSG00000231389 | ENSG00000226578 |
| ENSG00000186838 | ENSG00000275383 | ENSG00000176171 |
| ENSG00000187608 | ENSG00000259155 | ENSG00000273248 |
| ENSG00000222078 | ENSG00000250056 | ENSG00000261150 |
| ENSG00000227589 | ENSG00000003137 | ENSG00000165695 |
| ENSG00000081985 | ENSG00000224331 | ENSG00000242163 |
| ENSG00000236939 | ENSG00000171815 | ENSG00000146021 |
| ENSG00000234465 | ENSG00000250989 | ENSG00000280214 |
| ENSG00000154975 | ENSG00000099365 | ENSG00000270571 |
| ENSG00000104967 | ENSG00000169508 | ENSG00000115252 |
| ENSG00000109452 | ENSG00000149527 | ENSG00000180353 |
| ENSG00000164283 | ENSG00000162551 | ENSG00000225573 |
| ENSG00000224383 | ENSG00000257624 | ENSG00000185215 |
| ENSG00000241229 | ENSG00000106829 | ENSG00000183696 |
| ENSG00000106025 | ENSG00000270722 | ENSG00000108846 |
| ENSG00000132840 | ENSG00000142677 | ENSG00000266274 |
| ENSG00000131910 | ENSG00000223695 | ENSG00000227560 |
| ENSG00000145649 | ENSG00000046604 | ENSG00000105929 |
| ENSG00000258505 | ENSG00000277504 | ENSG00000242970 |
| ENSG00000003096 | ENSG00000279415 | ENSG00000232756 |
| ENSG00000236423 | ENSG00000023171 | ENSG00000168389 |
| ENSG00000260468 | ENSG00000161328 | ENSG00000258454 |
| ENSG00000279878 | ENSG00000204397 | ENSG00000279255 |
| ENSG00000197653 | ENSG00000269919 | ENSG00000256546 |
| ENSG00000251301 | ENSG00000268975 | ENSG00000224905 |
| ENSG00000240964 | ENSG00000151632 | ENSG00000279543 |
| ENSG00000237359 | ENSG00000248206 | ENSG00000105855 |
| ENSG00000267767 | ENSG00000201129 | ENSG00000117318 |
| ENSG00000228848 | ENSG00000258611 | ENSG00000121314 |
| ENSG00000265052 | ENSG00000134594 | ENSG00000149090 |
| ENSG00000090376 | ENSG00000206834 | ENSG00000141756 |
| ENSG00000122584 | ENSG00000274427 | ENSG00000279499 |
| ENSG00000119630 | ENSG00000100167 | ENSG00000187867 |
| ENSG00000166922 | ENSG00000242829 | ENSG00000110077 |
| ENSG00000204963 | ENSG00000183397 | ENSG00000113140 |
| ENSG00000233052 | ENSG00000152785 | ENSG00000109832 |
| ENSG00000259855 | ENSG00000104419 | ENSG00000069011 |
| ENSG00000266711 | ENSG00000245848 | ENSG00000228477 |
| ENSG00000213183 | ENSG00000266909 | ENSG00000204131 |
| ENSG00000108821 | ENSG00000139865 | ENSG00000233060 |
| ENSG00000214243 | ENSG00000177570 | ENSG00000254270 |
| ENSG00000237668 | ENSG00000272431 | ENSG00000112186 |
| ENSG00000101213 | ENSG00000164932 | ENSG00000278934 |
| ENSG00000178162 | ENSG00000275613 | ENSG00000239528 |
| ENSG00000177839 | ENSG00000091129 | ENSG00000233108 |
| ENSG00000196263 | ENSG00000211747 | ENSG00000154262 |
| ENSG00000169136 | ENSG00000126785 | ENSG00000179082 |
| ENSG00000206903 | ENSG00000128591 | ENSG00000242951 |
| ENSG00000163737 | ENSG00000081923 | ENSG00000141526 |
| ENSG00000216316 | ENSG00000224796 | ENSG00000160229 |
| ENSG00000233836 | ENSG00000144115 | ENSG00000159708 |

In some examples, a digital pathology platform (e.g., a digital pathology platform as described herein, e.g., in Section IV below) may be used to assign a patient's tumor to a subtype as disclosed herein. The molecular subtype of the SCLC tumor sample may be determined in conjunction with or in the absence of patient tumor-specific transcriptome data. For example, in some cases, the molecular subtype of the SCLC tumor sample may be determined in conjunction with patient tumor-specific transcriptome data. In other cases, the molecular subtype of the SCLC tumor sample may be determined in the absence of patient tumor-specific transcriptome data.

For example, provided herein is a method of classifying an SCLC in a human patient, the method comprising: (a) assaying an image of a tumor sample from the patient using a digital pathology system; and (b) the patient's tumor sample into one of the following four subtypes based on the transcriptional profile of the patient's tumor: NE-I, NE-N, NE-A, or nNE-I, thereby classifying the SCLC in the patient.

Any of the methods disclosed herein may further include determining the expression level (e.g., the mRNA expression level) of one or more genes or gene signatures.

In some examples, the method further comprises determining the mRNA expression level of one or more of the following gene signatures in the tumor sample from the patient: (a) a neuroendocrine (NE) signature comprising one or more (e.g., one, two, three, or four), or all, of CHGA, DLL3, NEUROD1, INSM1, and ASCL1; (b) a non-NE signature comprising one or more (e.g., one, two, or three), or all, of YAP1, POU2F3, MYC, and REST; (c) an endothelial-mesenchymal transition (EMT) signature comprising one or more (e.g., one, two, or three), or all, of ZEB1, ZEB2, SNAI1, and TWIST1; (d) a T-effector (T-eff) signature comprising one or more (e.g., one, two, three, four, five, six, or seven), or all, of CD8A, GZBA, GZMB, PRF1, IFNG, CXCL9, CXCL10, and TBX21; (e) a B/plasma cell (B/PC) signature comprising one or more (e.g., one, two, or three), or all, of CD79A, MS4A1, MZB1, and JCHAIN; (f) an antigen-presenting machinery (APM) signature comprising one or more (e.g., one, two, three, or four), or all, of TAP1, TAP2, B2M, HLA-A, and HLA-C; (g) a checkpoint signature comprising one or more (e.g., one, two, three, four, or five), or all, of PDCD1, CD274, LAG3, CTLA4, BTLA, and TIGIT; (h) an immune stimulatory signature comprising one or more (e.g., one, two, three, four, five, six, seven, eight, nine, 10, or 11), or all, of CD27, CD28, CD40, CD40LG, IL2RB, TNFRSF4, TNFSF4, ICOSLG, ICOS, TNFRSF18, TNFSF18, TNFRSF9, and TNFSF9; (i) an immune inhibitory signature comprising one or more (e.g., one, two, three, four, five, six, seven, eight, nine, 10, 11, 12, 13, 14, 15, 16, 17, 18, or 19), or all, of CD274, PDCD1, PDCD1LG2, CTLA4, CD86, CD80, CD200, CD200R1, VSIR, IGSF11, LAG3, CLEC4G, BTLA, CD160, TNFRSF14, HAVCR2, CEACAM1, HMGB1, LGALS9, and TIGIT; (j) a general myeloid signature comprising one or more (e.g., one, two, three, four, five, six, seven, eight, or nine), or all, of CLEC9A, LAMP3, CD68, MRC1, TGM2, NOS2, SOCS3, CD163, FCGR3A, and FCGR3B; (k) an angiogenesis signature comprising one or more (e.g., one, two, three, four, or five), or all, of VEGFA, KDR, ESM1, PECAM1, ANGPTL4, and CD34; (I) a tumor-associated macrophage signature comprising one or more (e.g., one, two, three, four, five, six, seven, eight, nine, 10, 11, 12, 13, 14, 15), or all, of MARCO, ACP5, VSIG4, MRC1, MSR1, MCEMP1, CYP27A1, OLR1, GRN, GLIPR2, ARRDC4, C1QC, APOE, FOLR2, CTSD, and SPP1; (m) a ciliated cell signature comprising one or both of C9orf24 and C20orf85; (n) a basal cell signature comprising one or more (e.g., one or two), or all, of TP63, KRT15, and KRT17; and/or (o) A goblet cell signature comprising one or both of SLC5A5 and SAA1.

In some examples, the patient's tumor sample is assigned into the NE-I subtype, and the patient's tumor sample has an increased expression level, relative to a reference expression level, of the neuroendocrine signature, the T-eff signature, the B/PC signature, the checkpoint signature, the APM signature, the immune stimulatory signature, the immune inhibitory signature, the general myeloid signature, the ciliated cell signature, the basal cell signature, and/or the goblet cell signature.

In some examples, the patient's tumor sample is assigned into the nNE-I subtype, and the patient's tumor sample has an increased expression level, relative to a reference expression level, of the T-eff signature, the B/PC signature, the checkpoint signature, the APM signature, the immune stimulatory signature, the immune inhibitory signature, and/or the general myeloid signature.

Any suitable reference expression level for a signature may be used. In some examples, the reference expression level is determined from a population of patients having a lung cancer (e.g., a SCLC, e.g., ES-SCLC or LS-SCLC, including in the 1L treatment setting). In some examples, the reference expression level of a signature is the median Z-score of the signature in a population of patients having an SCLC (e.g., ES-SCLC or LS-SCLC).

In some examples, the patient's tumor sample is assigned into the NE-I subtype, and the patient's tumor sample has: (i) an increased expression level, relative to a reference expression level, of ASCL1 or YAP1; (ii) an increased expression level, relative to a reference expression level, of the TGF beta signaling, p53 pathway, EMT, or NOTCH signaling MSigDB hallmark signatures; (iii) a decreased expression level, relative to a reference expression level, of the MYC targets MSigDB hallmark signature; and/or (iv) an increased expression level, relative to a reference expression level, of PD-L1 in tumor-infiltrating immune cells. In some examples, (i) the reference expression level of the TGF beta signaling, p53 pathway, EMT, or NOTCH signaling MSigDB hallmark signature is a median expression level of the TGF beta signaling, p53 pathway, EMT, or NOTCH signaling MSigDB hallmark signature in a population of patients having an SCLC; or (ii) the reference expression level of the MYC targets MSigDB hallmark signature is a median expression level of the MYC targets MSigDB hallmark signature in a population of patients having an SCLC.

In some examples, the patient's tumor sample is assigned into the NE-I subtype, and the patient's tumor sample has: (i) an increased expression level, relative to a reference expression level, of a T-eff signature comprising CD8A, GZBA, GZMB, PRF1, IFNG, CXCL9, CXCL10, and TBX21; and (ii) a decreased expression level, relative to a reference expression level, of a tumor-associated macrophage (TAM) signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1.

In some examples, the patient's tumor sample is assigned into the NE-I subtype, and the patient's tumor sample has an elevated expression level, relative to a reference expression level, of a ciliated cell signature comprising C9orf24 and C20orf85, a basal cell signature comprising TP63, KRT15, and KRT17, and/or a goblet cell signature comprising SLC5A5 and SAA1. In some examples, the reference expression level is the expression level of the ciliated cell signature, the basal cell signature, and/or the goblet cell signature in a population of SCLC patients whose tumor sample are assigned to the nNE-I subtype.

In some examples, the patient's tumor sample is assigned into the nNE-I subtype, and the patient's tumor sample has: (i) an increased expression level, relative to a reference expression level, of ASCL1, YAP1, POU2F3, REST, and/or MYC; (ii) an increased expression level, relative to a reference expression level, of the MYC targets MSigDB hallmark signature; (iii) a decreased expression level, relative to a reference expression level, of the G2M checkpoint, SHH signaling, mitotic spindle, spermatogenesis, and/or pancreas beta cells MSigDB hallmark signatures; and/or (iv) an increased expression level, relative to a reference expression level, of PD-L1 in tumor-infiltrating immune cells. In some examples, (i) the reference expression level of the MYC targets MSigDB hallmark signature is a median expression level of the MYC targets MSigDB hallmark signature in a population of patients having an SCLC; or (ii) the reference expression level of the G2M checkpoint, SHH signaling, mitotic spindle, spermatogenesis, or pancreas beta cells MSigDB hallmark signature is a median expression level of the G2M checkpoint, SHH signaling, mitotic spindle, spermatogenesis, or pancreas beta cells MSigDB hallmark signature in a population of patients having an SCLC.

In some examples, the patient's tumor sample is assigned into the nNE-I subtype, and the patient's tumor sample has: (i) an increased expression level, relative to a reference expression level, of a T-eff signature comprising CD8A, GZBA, GZMB, PRF1, IFNG, CXCL9, CXCL10, and TBX21; and (ii) an increased expression level, relative to a reference expression level, of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1. In some examples, the reference expression level for the TAM signature is the expression level of the TAM signature in a population of SCLC patients whose tumor samples are assigned to the NE-I subtype.

In some examples, the patient's tumor sample is assigned into the NE-A subtype, and the patient's tumor sample has: (i) an increased expression level, relative to a reference expression level, of ASCL1; and/or (ii) a decreased expression level, relative to a reference expression level, of TGF beta signaling, p53 pathway, EMT, NOTCH signaling, MYC targets, and/or WNT signaling MSigDB hallmark signatures. In some examples, the reference expression level of the TGF beta signaling, p53 pathway, EMT, NOTCH signaling, MYC targets, or WNT signaling MSigDB hallmark signature is a median expression level of the TGF beta signaling, p53 pathway, EMT, NOTCH signaling, MYC targets, or WNT signaling MSigDB hallmark signature in a population of patients having an SCLC.

In some examples, the patient's tumor sample is assigned into the NE-N subtype, and the patient's tumor sample has: (i) an increased expression level, relative to a reference expression level, of NEUROD1; and/or (ii) an increased expression level, relative to a reference expression level, of the DNA repair, MYC targets, WNT signaling, G2M checkpoint, SHH signaling, mitotic spindle, and/or spermatogenesis MSigDB hallmark signatures. In some examples, the reference expression level of the DNA repair, MYC targets, WNT signaling, G2M checkpoint, SHH signaling, mitotic spindle, or spermatogenesis MSigDB hallmark signature is a median expression level of the DNA repair, MYC targets, WNT signaling, G2M checkpoint, SHH signaling, mitotic spindle, or spermatogenesis MSigDB hallmark signature in a population of patients having an SCLC.

In another example, provided herein is a method of identifying a patient having a lung cancer (e.g., SCLC, e.g., ES-SCLC or LS-SCLC), including in the 1L treatment setting) who is likely to benefit from an anti-cancer therapy comprising a PD-1 axis binding antagonist (e.g., atezolizumab or avelumab), the method comprising: determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising the PD-1 axis binding antagonist (e.g., atezolizumab or avelumab).

In another example, provided herein is a method of selecting a therapy for a patient having a lung cancer (e.g., SCLC, e.g., ES-SCLC or LS-SCLC), including in the 1L treatment setting), the method comprising: (a) determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising a PD-1 axis binding antagonist (e.g., atezolizumab or avelumab); and (b) selecting an anti-cancer therapy comprising the PD-1 axis binding antagonist (e.g., atezolizumab or avelumab) for the patient identified as one who is likely to benefit from the anti-cancer therapy.

In some examples, the reference expression level for the T-eff signature is the median expression level of the T-eff signature in a population of patients having SCLC. In some examples, the reference expression level for the TAM is the median expression level of the TAM signature in a population of patients having SCLC.

In some examples, the patient's tumor sample is assigned into the NE-A subtype or the NE-N subtype, and the method further comprises treating the patient by administering to the patient a DNA damage response (DDR)-targeting agent. In some examples, the DDR-targeting agent is an anti-delta-like ligand 3 (DLL3) antibody-drug conjugate (ADC) (e.g., Rova-T) or an anti-DLL3 bispecific T cell engager (BiTE) (e.g., AMG 757).

In some examples, the patient's tumor sample is assigned into the nNE-I subtype, and the method further comprises treating the patient by administering to the patient a myeloid repolarization agent or a REST-targeted therapy. In some examples, the myeloid repolarization agent comprises a Toll-like receptor 7 (TLR7) agonist.

In some examples, assignment of the patient's tumor sample into the NE-I subtype indicates that the patient is likely to have an increased clinical benefit from treatment with an anti-cancer therapy comprising a PD-1 axis binding antagonist (e.g., atezolizumab or avelumab) compared to a treatment that does not comprise a PD-1 axis binding antagonist (e.g., atezolizumab or avelumab). In some examples, assignment of the patient's tumor sample into the NE-I subtype indicates that the patient is likely to have an increased clinical benefit from treatment with an anti-cancer therapy comprising atezolizumab compared to a treatment that does not comprise atezolizumab. In some examples, assignment of the patient's tumor sample into the NE-I subtype indicates that the patient is likely to have an increased clinical benefit from treatment with an anti-cancer therapy comprising avelumab compared to a treatment that does not comprise avelumab. In some examples, the treatment that does not comprise atezolizumab comprises a chemotherapeutic agent (e.g., carboplatin and etoposide) or observation. In some examples, increased clinical benefit comprises a relative increase in one or more of the following: overall survival (OS), objective response rate (ORR), progression-free survival (PFS), complete response (CR), partial response (PR), or a combination thereof. In some examples, increased clinical benefit comprises a relative increase in OS.

In some examples, the patient's tumor sample is assigned into the NE-I subtype, and the method further comprises selecting an anti-cancer therapy comprising a PD-1 axis binding antagonist (e.g., atezolizumab or avelumab) or a CTLA-4 antagonist (e.g., an anti-CTLA4 antibody) for the patient. In some examples, the method further comprises selecting an anti-cancer therapy comprising atezolizumab. In other examples, the method further comprises selecting an anti-cancer therapy comprising avelumab.

In some examples, the patient's tumor sample is assigned into the NE-I subtype, and the method further comprises treating the patient by administering an anti-cancer therapy comprising a PD-1 axis binding antagonist (e.g., atezolizumab or avelumab) or a CTLA-4 antagonist (e.g., an anti-CTLA4 antibody) to the patient. In some examples, the method further comprises treating the patient by administering an anti-cancer therapy comprising atezolizumab to the patient. In other examples, the method further comprises treating the patient by administering an anti-cancer therapy comprising avelumab to the patient.

In some examples, the patient's tumor sample is assigned into the NE-I subtype, and the method further comprises selecting an anti-cancer therapy comprising a PD-1 axis binding antagonist (e.g., atezolizumab or avelumab) in combination with one or more additional immunotherapy agents (e.g., a cluster of differentiation 28 (CD28) agonist, an OX40 agonist, a glucocorticoid-induced TNFR-related (GITR) agonist, a cluster of differentiation 137 (CD137) agonist, a cluster of differentiation 27 (CD27) agonist, an inducible T-cell costimulator (ICOS) agonist, a herpes virus entry mediator (HVEM) agonist, a natural killer group 2 member D (NKG2D) agonist, a MHC class I polypeptide-related sequence A (MICA) agonist, a natural killer cell receptor 2B4 agonist, a PD-1 axis binding antagonist, a CTLA4 antagonist, a TIM3 antagonist, a B and T lymphocyte associated (BTLA) antagonist, a V-domain Ig suppressor of T cell activation (VISTA) antagonist, a LAG3 antagonist, a B7-H4 antagonist, a cluster of differentiation 96 (CD96) antagonist, a TIGIT antagonist, a cluster of differentiation 226 (CD226) antagonist, a chemokine receptor 8 (CCR8) antagonist, a cancer vaccine, an adoptive cell therapy, or a combination thereof) for the patient.

In some examples, the patient's tumor sample is assigned into the NE-I subtype, and the method further comprises treating the patient by administering to the patient a PD-1 axis binding antagonist (e.g., atezolizumab or avelumab) in combination with one or more additional immunotherapy agents (e.g., a CD28 agonist, an OX40 agonist, a GITR agonist, a CD137 agonist, a CD27 agonist, an ICOS agonist, an HVEM agonist, an NKG2D agonist, a MICA agonist, a 2B4 agonist, a PD-1 axis binding antagonist, a CTLA4 antagonist, a TIM3 antagonist, a BTLA antagonist, a VISTA antagonist, a LAG3 antagonist, a B7-H4 antagonist, a CD96 antagonist, a TIGIT antagonist, a CD226 antagonist, a CCR8 antagonist, a cancer vaccine, an adoptive cell therapy, or a combination thereof).

In some examples, the immunotherapy agent is an immune checkpoint inhibitor. In some examples, the immunotherapy agent is a CD28, OX40, GITR, CD137, CD27, ICOS, HVEM, NKG2D, MICA, or 2B4 agonist or a CTLA-4, PD-1 axis, TIM-3, BTLA, VISTA, LAG-3, B7H4, CD96, TIGIT, or CD226 antagonist. Other particular immunotherapy agents that may be used include anti-CTLA-4 antibodies or antigen-binding fragments thereof, anti-CD27 antibodies or antigen-binding fragments thereof, anti-CD30 antibodies or antigen-binding fragments thereof, anti-CD40 antibodies or antigen-binding fragments thereof, anti-4-1BB antibodies or antigen-binding fragments thereof, anti-GITR antibodies or antigen-binding fragments thereof, anti-OX40 antibodies or antigen-binding fragments thereof, anti-TRAILR1 antibodies or antigen-binding fragments thereof, anti-TRAILR2 antibodies or antigen-binding fragments thereof, anti-TWEAK antibodies or antigen-binding fragments thereof, anti-TWEAKR antibodies or antigen-binding fragments thereof, anti-BRAF antibodies or antigen-binding fragments thereof, anti-MEK antibodies or antigen-binding fragments thereof, anti-CD33 antibodies or antigen-binding fragments thereof, anti-CD20 antibodies or antigen-binding fragments thereof, anti-CD52 antibodies or antigen-binding fragments thereof, anti-A33 antibodies or antigen-binding fragments thereof, anti-GD3 antibodies or antigen-binding fragments thereof, anti-PSMA antibodies or antigen-binding fragments thereof, anti-Ceacan 1 antibodies or antigen-binding fragments thereof, anti-Galedin 9 antibodies or antigen-binding fragments thereof, anti-HVEM antibodies or antigen-binding fragments thereof, anti-VISTA antibodies or antigen-binding fragments thereof, anti-B7 H4 antibodies or antigen-binding fragments thereof, anti-HHLA2 antibodies or antigen-binding fragments thereof, anti-CD155 antibodies or antigen-binding fragments thereof, anti-CD80 antibodies or antigen-binding fragments thereof, anti-BTLA antibodies or antigen-binding fragments thereof, anti-CD160 antibodies or antigen-binding fragments thereof, anti-CD28 antibodies or antigen-binding fragments thereof, anti-CD226 antibodies or antigen-binding fragments thereof, anti-CEACAM1 antibodies or antigen-binding fragments thereof, anti-TIM3 antibodies or antigen-binding fragments thereof, anti-CD96 antibodies or antigen-binding fragments thereof, anti-CD70 antibodies or antigen-binding fragments thereof, anti-CD27 antibodies or antigen-binding fragments thereof, anti-LIGHT antibodies or antigen-binding fragments thereof, anti-CD137 antibodies or antigen-binding fragments thereof, anti-DR4 antibodies or antigen-binding fragments thereof, anti-CR5 antibodies or antigen-binding fragments thereof, anti-FAS antibodies or antigen-binding fragments thereof, anti-CD95 antibodies or antigen-binding fragments thereof, anti-TRAIL antibodies or antigen-binding fragments thereof, anti-DR6 antibodies or antigen-binding fragments thereof, anti-EDAR antibodies or antigen-binding fragments thereof, anti-NGFR antibodies or antigen-binding fragments thereof, anti-OPG antibodies or antigen-binding fragments thereof, anti-RANKL antibodies or antigen-binding fragments thereof, anti-LTBR antibodies or antigen-binding fragments thereof, anti-BCMA antibodies or antigen-binding fragments thereof, anti-TACI antibodies or antigen-binding fragments thereof, anti-BAFFR antibodies or antigen-binding fragments thereof, anti-EDAR2 antibodies or antigen-binding fragments thereof, anti-TROY antibodies or antigen-binding fragments thereof, and anti-RELT antibodies or antigen-binding fragments thereof.

Any of the methods disclosed herein may comprise assaying for somatic alterations in the patient's genotype in the tumor sample obtained from the patient. Any suitable somatic alterations may be assayed. In some examples, the somatic alteration is a short variant, a loss, an amplification, a deletion, a duplication, a rearrangement, or a truncation.

Any suitable sample may be used for patient classification in the methods described herein. In some examples, the sample is a tumor sample. In some examples, the tumor sample is a formalin-fixed and paraffin-embedded (FFPE) sample, an archival sample, a fresh sample, or a frozen sample. In some examples, the tumor sample is a pre-treatment tumor sample.

In some examples, the patient has an ES-SCLC. In some examples, the patient has an LS-SCLC. In some examples, the patient is previously untreated for the SCLC. In some examples, the patient is chemotherapy-naïve.

In some examples, the PD-1 axis binding antagonist (e.g., atezolizumab or avelumab) is administered as a monotherapy. In some examples, the atezolizumab is administered as a monotherapy.

In some examples, the method further comprises selecting an additional therapeutic agent to the patient.

In some examples, the method further comprises administering an additional therapeutic agent to the patient.

In some examples, the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof. In some examples, the growth inhibitory agent is a CDK4/6 inhibitor (e.g., palbociclib, ribociclib, or abemaciclib). In some examples, the anti-angiogenic agent is a VEGF antagonist (e.g., any VEGF antagonist disclosed herein, e.g., an anti-VEGF antibody (e.g., bevacizumab) or a tyrosine kinase inhibitor (e.g., sunitinib or axitinib)) or a HIF2A inhibitor (e.g., belzutifan (also known as MK-6482) or PT2385). In some examples, the stromal inhibitor is a TGF-β antagonist (e.g., an anti-TGF-β antibody, e.g., any anti-TGF-β antibody disclosed herein). In some examples, the metabolism inhibitor is a PCSK9 inhibitor (e.g., an anti-PCSK9 antibody, e.g., alirocumab or evolocumab), a FAS inhibitor (e.g., cerulenin, C75, isoniazid, or orlistat (tetrahydrolipstatin)), or an AMPK inhibitor (e.g., SBI-0206965, 5′-hydroxy-staurosporine, or compound C (also known as dorsomorphin)). In some embodiments, the complement antagonist is a C1 inhibitor (e.g., CINRYZE® C1 esterase inhibitor), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB SIRNA, Alnylam), or an antisense (e.g., lonis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)).

Any of the methods of classifying a lung cancer in a patient may further include treating the patient, e.g., using any approach described below in Section III.

### III. Therapeutic Methods, Compositions, and Uses for Lung Cancer

In one example, provided herein is a method of treating a lung cancer (e.g., ES-SCLC or LS-SCLC), including in the 1L treatment setting) in a human patient, the method comprising: classifying the lung cancer in the patient according to any one of the methods disclosed herein; and administering an anti-cancer therapy to the patient based on the classification (e.g., into a subtype as disclosed herein).

In another example, provided herein is an anti-cancer therapy for use in treating a lung cancer (e.g., SCLC, e.g., ES-SCLC or LS-SCLC, including in the 1L treatment setting) in a human patient, wherein the SCLC in the patient has been classified (e.g., into a subtype as disclosed herein) according to any one of the methods disclosed herein.

In another example, provided herein is the use of an anti-cancer therapy in the preparation of a medicament for treating a lung cancer (e.g., SCLC, e.g., ES-SCLC or LS-SCLC, including in the 1L treatment setting) in a human patient, wherein the SCLC in the patient has been classified (e.g., into a subtype as disclosed herein) according to any one of the methods disclosed herein.

In some examples, the patient is previously untreated for the lung cancer, e.g., SCLC, e.g., ES-SCLC or LS-SCLC. In some examples, the patient has received a previous treatment for the lung cancer, e.g., SCLC, e.g., ES-SCLC or LS-SCLC.

For example, provided herein is a method of treating a lung cancer (e.g., SCLC, e.g., ES-SCLC or LS-SCLC, including in the 1L treatment setting) in a human patient, wherein the patient is previously untreated for the SCLC, the method comprising: classifying the cancer in the patient according to any one of the methods disclosed herein; and administering an anti-cancer therapy to the patient based on the classification (e.g., into a subtype as disclosed herein).

In another example, provided herein is an anti-cancer therapy for use in treating a lung cancer, e.g., SCLC (e.g., ES-SCLC or LS-SCLC) in a human patient, wherein the patient is previously untreated for the SCLC, wherein the SCLC in the patient has been classified (e.g., into a subtype as disclosed herein) according to any one of the methods disclosed herein.

In another example, provided herein is the use of an anti-cancer therapy in the preparation of a medicament for treating a lung cancer, e.g., SCLC (e.g., ES-SCLC or LS-SCLC) in a human patient, wherein the patient is previously untreated for the SCLC, wherein the SCLC in the patient has been classified (e.g., into a subtype as disclosed herein) according to any one of the methods disclosed herein.

In one example, provided herein is a method of treating an ES-SCLC in a human patient, wherein the patient is previously untreated for the ES-SCLC, the method comprising: classifying the previously untreated ES-SCLC in the patient according to any one of the methods disclosed herein; and administering an anti-cancer therapy to the patient based on the classification (e.g., into a subtype as disclosed herein).

In another example, provided herein is an anti-cancer therapy for use in treating an ES-SCLC in a human patient, wherein the patient is previously untreated for the ES-SCLC, and wherein the previously untreated ES-SCLC in the patient has been classified (e.g., into a subtype as disclosed herein) according to any one of the methods disclosed herein.

In another example, provided herein is the use of an anti-cancer therapy in the preparation of a medicament for treating an ES-SCLC in a human patient, wherein the patient is previously untreated for the ES-SCLC, and wherein the previously untreated ES-SCLC in the patient has been classified (e.g., into a subtype as disclosed herein) according to any one of the methods disclosed herein.

In another example, provided herein is a method of treating a patient having a lung cancer, e.g., an SCLC (e.g., an ES-SCLC or LS-SCLC), the method comprising: (a) determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising a PD-1 axis binding antagonist (e.g., an anti-PD-L1 antibody, e.g., atezolizumab); and (b) administering an anti-cancer therapy comprising the PD-1 axis binding antagonist (e.g., anti-PD-L1 antibody, e.g., atezolizumab) to the patient identified as one who is likely to benefit from the anti-cancer therapy.

In another example, provided herein is a method of treating a patient having a lung cancer, e.g., an SCLC (e.g., an ES-SCLC or LS-SCLC), the method comprising administering an anti-cancer therapy comprising a PD-1 axis binding antagonist (e.g., an anti-PD-L1 antibody, e.g., atezolizumab) to the patient, wherein the patient has been determined to have an increased expression level, relative to a reference expression level, of a T-eff signature comprising CD8A, GZBA, GZMB, PRF1, IFNG, CXCL9, CXCL10, and TBX21 and a decreased expression level, relative to a reference expression level, of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1 in a tumor sample from the patient.

Any suitable anti-cancer therapy may be administered to the patient based on the classification (e.g., into a subtype as disclosed herein). For example, in some embodiments, a PD-1 axis binding antagonist (e.g., an anti-PD-L1 antibody, e.g., atezolizumab or avelumab) is administered to the patient. In some examples, the anti-cancer therapy comprises atezolizumab. In other examples, the anti-cancer therapy comprises avelumab. In some examples, the anti-cancer therapy further comprises carboplatin and etoposide. In some examples, the method further comprises administering an additional therapeutic agent to the patient.

In some examples, the PD-1 axis binding antagonist is administered in combination with an effective amount of one or more additional therapeutic agents. In some examples, the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof. In some examples, the additional therapeutic agent is a DNA damage response (DDR)-targeting agent. In some examples, the additional therapeutic agent is a myeloid repolarization agent or a REST-targeted therapy. In some examples, the growth inhibitory agent is a CDK4/6 inhibitor (e.g., palbociclib, ribociclib, or abemaciclib). In some examples, the anti-angiogenic agent is a VEGF antagonist (e.g., any VEGF antagonist disclosed herein, e.g., an anti-VEGF antibody (e.g., bevacizumab) or a tyrosine kinase inhibitor (e.g., sunitinib or axitinib)) or a HIF2A inhibitor (e.g., belzutifan (also known as MK-6482) or PT2385). In some examples, the stromal inhibitor is a TGF-β antagonist (e.g., an anti-TGF-β antibody, e.g., any anti-TGF-β antibody disclosed herein). In some examples, the metabolism inhibitor is a PCSK9 inhibitor (e.g., an anti-PCSK9 antibody, e.g., alirocumab or evolocumab), a FAS inhibitor (e.g., cerulenin, C75, isoniazid, or orlistat (tetrahydrolipstatin)), or an AMPK inhibitor (e.g., SBI-0206965, 5′-hydroxy-staurosporine, or compound C (also known as dorsomorphin)). In some embodiments, the complement antagonist is a C1 inhibitor (e.g., CINRYZE® C1 esterase inhibitor), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., lonis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)). In some examples, the DDR-targeting agent is an anti-delta-like ligand 3 (DLL3) antibody-drug conjugate (ADC) (e.g., Rova-T) or an anti-DLL3 bispecific T cell engager (BiTE) (e.g., AMG 757). In some examples, the myeloid repolarization agent is a Toll-like receptor 7 (TLR7) agonist.

In any of the preceding examples, each dosing cycle may have any suitable length, e.g., about 7 days, about 14 days, about 21 days, about 28 days, about 35 days, about 42 days, or longer. In some instances, each dosing cycle is about 21 days. In some instances, each dosing cycle is about 42 days.

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In one instance, a PD-1 axis binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-1 axis binding antagonist may be administered at a dose of about 1000 mg to about 1400 mg every three weeks (e.g., about 1100 mg to about 1300 mg every three weeks, e.g., about 1150 mg to about 1250 mg every three weeks). In some instances, the PD-1 axis binding antagonist may be administered at a dose of 840 mg every two weeks. In some instances, the PD-1 axis binding antagonist may be administered at a dose of 1200 mg every three weeks. In some instances, the PD-1 axis binding antagonist may be administered at a dose of 1680 mg every four weeks.

In some instances, a patient is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

In some instances, atezolizumab is administered to the patient intravenously at a dose of about 840 mg every 2 weeks (Q2 W), about 1200 mg every 3 weeks (Q3 W), or about 1680 mg of every 4 weeks (Q4 W). In some instances, atezolizumab is administered to the patient intravenously at a dose of 840 mg every two weeks (Q2 W), 1200 mg every three weeks (Q3 W), or 1680 mg every four weeks (Q4 W). In some instances, atezolizumab is administered to the patient intravenously at a dose of about 840 mg every 2 weeks. In some instances, atezolizumab is administered to the patient intravenously at a dose of about 1200 mg every 3 weeks. In some instances, atezolizumab is administered to the patient intravenously at a dose of about 1680 mg of every 4 weeks.

In some instances, atezolizumab is administered at a fixed dose of 1200 mg via intravenous infusion on Day 1 of each 21-day cycle.

In some instances, avelumab is administered at a dose of 10 mg/kg IV every two weeks.

The PD-1 axis binding antagonist and/or any additional therapeutic agent(s), including an immunotherapy agent, a cytotoxic agent, a growth inhibitory agent, a stromal inhibitor, a metabolism inhibitor, a complement antagonist, a radiation therapy agent, an anti-angiogenic agent (e.g., a VEGF antagonist), or a combination thereof, may be administered in any suitable manner known in the art.

For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the patient on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the anti-cancer therapy is administered to the patient in a dosing regimen comprising: (i) an induction phase comprising four 21-day cycles, wherein atezolizumab is administered to the patient at a dose of 1200 mg intravenously (IV) on Day 1 of each cycle, carboplatin is administered to the patient at an initial target area under the curve (AUC) of 5 mg/mL/min IV on Day 1 of each cycle, and etoposide is administered to the patient at a dose of 100 mg/m² IV on Days 1, 2, and 3 of each cycle; and (ii) a maintenance phase comprising one or more 21-day cycles, wherein atezolizumab is administered to the patient at a dose of 1200 mg IV on Day 1 of each 21-day cycle.

In some examples, the maintenance phase continues until persistent radiographic progressive disease (PD), symptomatic deterioration, intolerable toxicity, or death.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

Also provided herein are methods for treating lung cancer, e.g., SCLC (e.g., ES-SCLC or LS-SCLC) in a patient comprising administering to the patient a treatment regimen comprising an effective amount of a PD-1 axis binding antagonist (e.g., atezolizumab) and/or in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, etoposide, cisplatin, pemetrexed, gemcitabine, cobimetinib, vemurafenib, or a combination thereof. The PD-1 axis binding antagonist may be an anti-PD-L1 antibody (e.g., atezolizumab) or an anti-PD-1 antibody.

For example, when administering with chemotherapy, atezolizumab may be administered at a dose of 1200 mg every 3 weeks prior to chemotherapy. In another example, following completion of 4-6 cycles of chemotherapy, atezolizumab may be administered at a dose of 840 mg every 2 weeks, 1200 mg every 3 weeks, or 1680 mg every four weeks. In another example, atezolizumab may be administered at a dose of 840 mg, followed by 100 mg/m² of paclitaxel protein-bound (e.g., nab-paclitaxel); for each 28 day cycle, atezolizumab is administered on days 1 and 15, and paclitaxel protein-bound is administered on days 1, 8, and 15. In another example, when administering with carboplatin and etoposide, atezolizumab can be administered at a dose of 1200 mg every 3 weeks prior to chemotherapy. In yet another example, following completion of 4 cycles of carboplatin and etoposide, atezolizumab may be administered at a dose of 840 mg every 2 weeks, 1200 mg every 3 weeks, or 1680 mg every 4 weeks.

Therapeutically effective amounts of various chemotherapeutic agents are known in the art and contemplated in the present invention. In particular instances, one or more chemotherapeutic agents (e.g., a platinum-based chemotherapeutic agent (e.g., carboplatin or cisplatin) and/or a topoisomerase II inhibitor (e.g., etoposide)) are administered according to the doses recited herein. In some instances, the effective amount of a platinum-based chemotherapeutic agent (e.g., carboplatin or cisplatin) is a dose sufficient to achieve an AUC from 1-50 mg/ml/min (e.g., 2-25 mg/ml/min, 3-15 mg/ml/min, 4-10 mg/ml/min, or 5 mg/ml/min, e.g., 2 mg/ml/min, 3 mg/ml/min, 4 mg/ml/min, 5 mg/ml/min, 6 mg/ml/min, 7 mg/ml/min, 8 mg/ml/min, 9 mg/ml/min, 10 mg/ml/min, 11 mg/ml/min, 12 mg/ml/min, 13 mg/ml/min, 14 mg/ml/min, 15 mg/ml/min, 20 mg/ml/min, 25 mg/ml/min, 30 mg/ml/min, 35 mg/ml/min, 40 mg/ml/min, 45 mg/ml/min, 50 mg/ml/min). In some instances, the effective amount of the platinum-based chemotherapeutic agent (e.g., carboplatin or cisplatin) is a dose sufficient to achieve an AUC=5 mg/ml/min. In some instances, the effective amount of the platinum-based chemotherapeutic agent (e.g., carboplatin or cisplatin) is a dose sufficient to achieve an AUC=6 mg/ml/min. In some instances, the effective amount of carboplatin is a dose sufficient to achieve an AUC=5 mg/ml/min. In some instances, the effective amount of carboplatin is a dose sufficient to achieve an AUC=6 mg/ml/min. In some instances, the effective amount of carboplatin is a dose sufficient to achieve an AUC=5 mg/ml/min on Day 1 of a 21-day dosing cycle. In some instances, the effective amount of carboplatin is a dose sufficient to achieve an AUC=6 mg/ml/min on Day 1 of a 21-day dosing cycle.

In some instances, AUC can be calculated using the Calvert formula (Calvert et al., J. Clin. Oncol. 1989, 7:1748-56):

$$Total dose ( mg ) = ( target AUC ) \times ( g lomerular filtration rate [ GFR ] + 25 )$$

In some instances, the effective amount of the platinum-based chemotherapeutic agent (e.g., carboplatin or cisplatin) is 200 mg-1500 mg (e.g., 300 mg-1200 mg, 400 mg-1100 mg, or 500 mg-1000 mg, e.g., 300 mg-400 mg, 400 mg-500 mg, 500 mg-600 mg, 600 mg-700 mg, 700 mg-750 mg, 750 mg-800 mg, 800 mg-900 mg, 900 mg-1000 mg, 1000 mg-1100 mg, or 1100 mg-1200 mg, e.g., about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg). In some instances, the effective amount of the platinum-based chemotherapeutic agent (e.g., carboplatin or cisplatin) is about 500 mg-1000 mg (e.g., about 500 mg, about 600 mg, about 700 mg, about 800 mg, about 900 mg, or about 1000 mg).

In some instances, the effective amount of the platinum-based chemotherapeutic agent (e.g., carboplatin or cisplatin) is between about 20 mg/m² to about 200 mg/m² (e.g., between about 20 mg/m² to about 150 mg/m², e.g., between about 30 mg/m² to about 125 mg/m², e.g., between about 40 mg/m² to about 110 mg/m², e.g., between about 50 mg/m2 to about 100 mg/m², e.g., between about 60 mg/m2 to about 90 mg/m², e.g., between about 70 mg/m2 to about 80 mg/m², e.g., about 75 mg/m², e.g., 75 mg/m2). In some instances, the effective amount of the platinum-based chemotherapeutic agent (e.g., carboplatin or cisplatin) is about 75 mg/m². In some instances, the effective amount of cisplatin is about 75 mg/m². In some instances, the effective amount of cisplatin is about 75 mg/m2 every three weeks. In some instances, the effective amount of the platinum-based chemotherapeutic agent (e.g., carboplatin or cisplatin) is between 20 mg/m2 to 200 mg/m2 (e.g., between 20 mg/m2 to 150 mg/m², e.g., between 30 mg/m2 to 125 mg/m², e.g., between 40 mg/m2 to 110 mg/m², e.g., between 50 mg/m2 to 100 mg/m², e.g., between 60 mg/m2 to 90 mg/m², e.g., between 70 mg/m2 to 80 mg/m², e.g., 75 mg/m², e.g., 75 mg/m2). In some instances, the effective amount of the platinum-based chemotherapeutic agent (e.g., carboplatin or cisplatin) is 75 mg/m². In some instances, the effective amount of cisplatin is 75 mg/m². In some instances, the effective amount of cisplatin is 75 mg/m2 every three weeks.

In some instances, the platinum-based chemotherapeutic agent (e.g., carboplatin or cisplatin) is administered to the subject or population of subjects intravenously (e.g., over a 30-120-minute infusion). In some instances, carboplatin is administered intravenously over a 30-60-minute infusion. In some instances, cisplatin is administered intravenously over a 60-120-minute infusion. In some instances, the platinum-based chemotherapeutic agent (e.g., carboplatin or cisplatin) is administered to the subject or population of subjects every three weeks. In some instances, the platinum-based chemotherapeutic agent (e.g., carboplatin or cisplatin) is administered to the subject or population of subjects on about Day 1 (e.g., Day −3, Day −2, Day −1, Day 1, Day 2, or Day 3) of a 21-day dosing cycle.

In some instances, the effective amount of a topoisomerase II inhibitor (e.g., etoposide) is from 10-1000 mg/m² (e.g., from 20-800 mg/m², from 30-700 mg/m², from 40-500 mg/m², from 50-300 mg/m², from 75-200 mg/m², or from 80-150 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², or about 1000 mg/m² (e.g., 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 250 mg/m², 300 mg/m², 400 mg/m², 500 mg/m², 600 mg/m², 700 mg/m², 800 mg/m², 900 mg/m², or 1000 mg/m²)). In some instances, the effective amount of the topoisomerase II inhibitor (e.g., etoposide) is about 100 mg/m². In some instances, the effective amount of the topoisomerase II inhibitor (e.g., etoposide) is about 100 mg/m² on Days 1, 2, and 3 of each 21-day cycle. In some instances, the effective amount of the topoisomerase II inhibitor (e.g., etoposide) is 100 mg/m² on Days 1, 2, and 3 of each 21-day cycle.

In some embodiments, the topoisomerase II inhibitor (e.g., etoposide) is administered to the subject intravenously (e.g., over a 60-minute infusion).

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### IV. Digital Pathology Platform

Also described herein is a digital pathology platform that may perform machine learning enabled image-based molecular subtype classification in which the molecular subtype of a tumor sample, such as a small cell lung cancer (SCLC) tumor sample, is determined by applying one or more machine learning models to an image of the tumor sample (e.g., a whole slide microscopic image and/or the like). For example, the one or more machine learning models may be trained to determine, based on morphological features present in the image of the SCLC tumor sample, the molecular subtype of the SCLC tumor sample. In some cases, the SCLC tumor sample may be classified, based on the image of the SCLC tumor, as exhibiting a neuroendocrine NEUROD1-driven (NE-N; NMF1) subtype, a neuroendocrine ASCL1-driven (NE-A; NMF2) subtype, a neuroendocrine inflamed (NE-I; NMF3) subtype, or a nonneuroendocrine inflamed (nNE-I; NMF4) subtype. Accordingly, in some embodiments, the molecular subtype of the SCLC tumor sample may be determined in conjunction with or in the absence of patient tumor-specific transcriptome data.

In some example embodiments, the digital pathology platform may determine the molecular subtype of a SCLC tumor sample based on one or more features extracted from an image of the SCLC tumor sample. For example, the one or more machine learning models may include a first machine learning model trained to identify one or more visible features present in the image of the SCLC tumor sample. As used herein, the term “visible features” may refer to features in an image that are capable of being identified, localized, interpreted, inferred, and/or otherwise detected through a visual inspection of the image, for example, by a human, a machine, an algorithm, and/or the like. Moreover, the one or more machine learning models may further include a second machine learning model trained to determine, based at least on the one or more visible features extracted from the image of the SCLC tumor sample, the molecular subtype of the SCLC tumor sample.

In some example embodiments, the one or more visible features extracted from the image of the SCLC tumor may include tumor cell-intrinsic features as well as tumor micro-environmental features observed in the image of the SCLC tumor. For example, in some cases, the first machine learning model may be trained to identify, within the image of the SCLC tumor sample, one or more tumor cells, tumor associated macrophages, B-cells, T-cells, ciliated cells, basal cells, goblet cells, and/or the like. The second machine learning model may subsequently determine the molecular subtype of SCLC tumor sample based on the quantity, proportion, and/or spatial distribution of the one or more tumor cells, tumor associated macrophages, B-cells, T-cells, ciliated cells, basal cells, goblet cells, and/or the like.

In some example embodiments, the digital pathology platform may apply an end-to-end model to determine, based at least on an image of the SCLC tumor sample, the molecular subtype of the SCLC tumor. For example, in some cases, the end-to-end model may be applied to the image of the SCLC tumor sample to determine the molecular subtype of the SCLC tumor sample without an intermediate extraction of any visible features from the image.

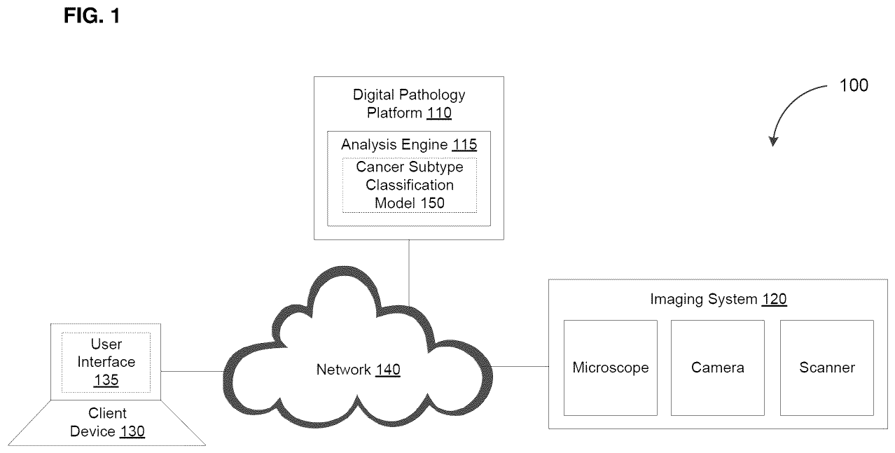

FIG. 1 depicts a system diagram illustrating an example of a digital pathology system 100, in accordance with some example embodiments. Referring to FIG. 1, the digital pathology system 100 may include a digital pathology platform 110, an imaging system 120, and a client device 130. As shown in FIG. 1, the digital pathology platform 110, the imaging system 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The imaging system 120 may include one or more imaging devices including, for example, a microscope, a digital camera, a whole slide scanner, a robotic microscope, and/or the like. The client device 130 may be a processor-based device including, for example, a workstation, a desktop computer, a laptop computer, a smartphone, a tablet computer, a wearable apparatus, and/or the like. Referring again to FIG. 1, the digital pathology platform 110 may include an analysis engine 115 configured to determine, based at least on one or more images of a tumor sample, one or more molecular subtypes associated with the tumor sample. The one or more images of the tumor sample may be whole slide images (WSI) received at digital pathology platform 110, for example, from the imaging system 120. In some cases, the tumor sample may be a SCLC tumor sample associated with a neuroendocrine NEUROD1-driven (NE-N; NMF1) subtype, a neuroendocrine ASCL1-driven (NE-A; NMF2) subtype, a neuroendocrine inflamed (NE-I; NMF3) subtype, or a nonneuroendocrine inflamed (nNE-I; NMF4) subtype.

The different molecular subtypes of the SCLC may be identified based on the transcriptome data of various SCLC tumor samples (e.g., a non-negative matrix factorization (NMF) e.g., as described herein, or other cluster analysis of the transcriptome data). The different molecular subtypes of SCLC may be associated with different lung cell lineages. Moreover, each molecular subtype of SCLC may present a unique combination of morphological features, including tumor cell-intrinsic features and tumor microenvironment (TME) features, that can be observed in the images (e.g., whole slide images and/or the like) of SCLC tumor samples.

Accordingly, in some example embodiments, the analysis engine 115 may train or apply a cancer subtype classification model 150 that determines, based at least on an image of a SCLC tumor sample, the molecular subtype of the SCLC tumor sample. In some cases, the cancer subtype classification model 150 may be trained based on annotated training data in which each training sample includes an image of a SCLC tumor sample and a ground-truth label of a molecular subtype determined based on a transcriptome data (e.g., RNA sequence data and/or the like) associated with the SCLC tumor sample. In some cases, the molecular subtype of the SCLC tumor sample may serve as a biomarker for predicting patient response to therapy, e.g., treatment with a PD-1 axis binding antagonist (e.g., atezolizumab (e.g., treatment with atezolizumab in combination with carboplatin and etoposide)), and survival. For example, the analysis engine 115 may determine or predict, based at least on an output of the cancer subtype classification model 150 indicating the molecular subtype of the SCLC tumor sample, a response of a patient associated with the SCLC tumor sample to a treatment for SCLC such as atezolizumab and/or the like. Treatment selection and planning for the patient associated with the SCLC tumor sample may be informed based on the patient's predicted response to certain treatments (e.g., a PD-1 axis binding antagonist (e.g., atezolizumab (e.g., atezolizumab in combination with carboplatin and etoposide)) and/or the like).

In some example embodiments, the cancer subtype classification model 150 may include a first machine learning model (e.g., an artificial neural network (ANN) and/or the like) trained to identify, within the image of the SCLC tumor sample, one or more visible features that are capable of being identified through a visual inspection of the image. For example, in some cases, the first machine learning model may be trained to segment the image of the SCLC tumor sample by at least assigning, to each pixel within the image, a label identifying the visible feature depicted by the pixel. Examples of visible features include one or more tumor cells, tumor associated macrophages, B-cells, T-cells, ciliated cells, basal cells, goblet cells, and/or the like. Moreover, the cancer subtype classification model 150 may include a second machine learning model (e.g., an artificial neural network (ANN) and/or the like) trained to determine, based at least on the one or more visible features identified within the image of the SCLC tumor sample, the molecular subtype of the SCLC tumor sample depicted in the image. For instance, in some cases, the second machine learning model may be trained to determine the molecular subtype of the SCLC tumor sample depicted in the image based on the quantity, proportion, and/or spatial distribution of the one or more tumor cells, tumor associated macrophages, B-cells, T-cells, ciliated cells, basal cells, goblet cells, and/or the like.

In some example embodiments, the cancer subtype classification model 150 may be implemented as an end-to-end machine learning model (e.g., an artificial neural network (ANN) and/or the like) trained to determine, based at least on an image of the SCLC tumor sample, the molecular subtype of the SCLC tumor. For example, the end-to-end machine learning model may be applied to the image of the SCLC tumor sample to determine the molecular subtype of the SCLC tumor sample without an intermediate extraction of any visible features from the image. The end-to-end machine learning model may determine the molecular subtype of the SCLC tumor sample by at least identifying one or more hidden features, which may not necessarily correspond to the aforementioned visible features.

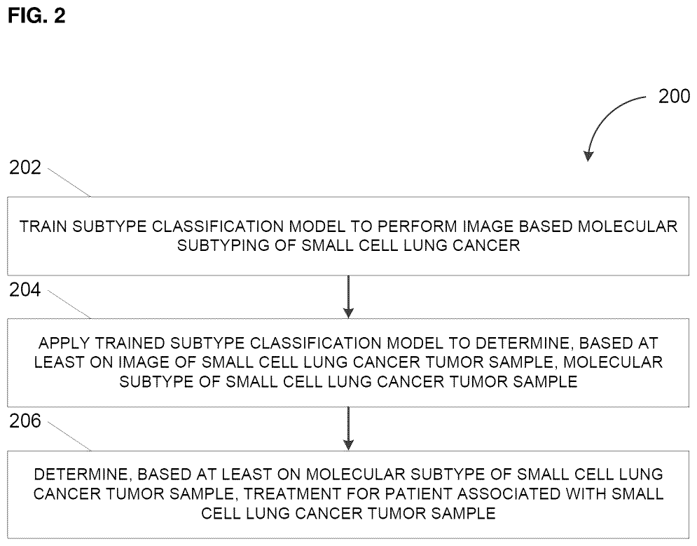

FIG. 2 depicts a flowchart illustrating an example of a process 200 for image-based molecular subtype classification, in accordance with some example embodiments. For instance, in some example embodiments, the analysis engine 115 at the digital pathology platform 110 may perform the process 200 to determine, based at least on an image of a SCLC tumor sample received from the imaging system 120, the molecular subtype of the SCLC tumor sample depicted in the image. In some cases, the analysis engine 115 may further perform the process 200 to determine, based at least on the molecular subtype of the SCLC tumor sample, a response of a patient associated with the tumor sample to certain treatments for SCLC such as atezolizumab and/or the like.

At 202, the analysis engine 115 may train the cancer subtype classification model 150 to perform image based molecular subtyping of SCLC. In some example embodiments, the analysis engine 115 may train, based at least on annotated training data, the cancer subtype classification model 150 to determine the molecular subtype of various SCLC tumor samples based on one or more corresponding images of the SCLC tumor samples. In cases where the cancer subtype classification model 150 is trained to determine the molecular subtype of a SCLC tumor sample based on visible features extracted from an image of the SCLC tumor sample, the annotated training data may include a first set of annotated training samples for training the first machine learning model and a second set of annotated training samples for training the second machine learning model. Each training sample in the first set of annotated training samples may include an image of a SCLC tumor sample and one or more ground truth labels of the visible features present in the image. Moreover, where the first machine learning model is being trained to segment the image of the SCLC tumor sample, each pixel in the image may be associated with a ground truth label identifying the visible feature depicted in the pixel. For the second set of annotated training samples, each training sample contained therein may include a combination of one or more visible features present in a SCLC tumor sample as well as a ground truth label of the corresponding molecular subtype. In some cases, the ground truth label of the molecular subtype may be determined and/or confirmed based on a transcriptome data associated with the SCLC tumor sample.

In instances where the cancer subtype classification model 150 is implemented as an end-to-end machine learning model without the intermediate extraction of one or more visible features, each training sample included in the annotated training data used to train the cancer subtype classification model 150 may include an image of a SCLC tumor sample and a ground truth label identifying the molecular subtype of the SCLC tumor sample. In some cases, the ground truth label assigned to an image of a SCLC tumor sample, which identifies the molecular subtype of the SCLC tumor sample, may be determined and/or confirmed based on a transcriptome data (e.g., RNA sequence data and/or the like) associated with the SCLC tumor sample.

At 204, the analysis engine 115 may apply the trained cancer subtype classification model 150 to determine, based at least on an image of a SCLC tumor sample, a molecular subtype of the SCLC tumor sample. In some example embodiments, the analysis engine 115 may apply the trained cancer subtype classification model 150 to determine, based at least on an image of a SCLC tumor sample received from the imaging system 120, a molecular subtype of the SCLC tumor sample. For example, the trained cancer subtype classification model 150 may be applied to determine whether the SCLC tumor sample depicted in the image exhibits a neuroendocrine NEUROD1-driven (NE-N; NMF1) subtype, a neuroendocrine ASCL1-driven (NE-A; NMF2) subtype, a neuroendocrine inflamed (NE-I; NMF3) subtype, or a nonneuroendocrine inflamed (nNE-I; NMF4) subtype.

In some cases, the trained cancer subtype classification model 150 may determine, based at least on the combination of visible features extracted from the image, the molecular subtype of the SCLC tumor sample depicted in the image. Examples of visible features extracted from the image of the SCLC tumor sample may include one or more tumor cells, tumor associated macrophages, B-cells, T-cells, ciliated cells, basal cells, goblet cells, and/or the like. The trained cancer subtype classification model 150 may determine the molecular subtype of the SCLC tumor sample depicted in the image based on the quantity, proportion, and/or spatial distribution of the one or more tumor cells, tumor associated macrophages, B-cells, T-cells, ciliated cells, basal cells, goblet cells, and/or the like. Alternatively, where the trained cancer subtype classification model 150 is implemented as an end-to-end machine learning model, the trained cancer subtype classification model 150 may determine the molecular subtype of the SCLC tumor sample depicted in the image based on a combination of hidden features.

At 206, the analysis engine 115 may determine or predict, based at least on the molecular subtype of the SCLC tumor sample, a treatment for a patient associated with the SCLC tumor sample. In some example embodiments, the analysis engine 115 may determine or predict, based at least on the molecular subtype of the SCLC tumor sample depicted in the image, a response of a patient associated with the SCLC tumor sample to certain treatments for SCLC. For example, in some cases, the molecular subtype exhibited by the SCLC tumor sample of the patient may be indicative of a likelihood of the patient responding to certain SCLC treatments such as a PD-1 axis binding antagonist (e.g., atezolizumab (e.g., atezolizumab in combination with carboplatin and etoposide)) and/or the like. Accordingly, in some cases, the analysis engine 115 may determine or predict, based at least on the molecular subtype of the SCLC tumor sample associated with the patient, a treatment plan for the patient. For example, the analysis engine 115 may determine or predict to include (or exclude) a certain treatment (e.g., a PD-1 axis binding antagonist (e.g., atezolizumab (e.g., atezolizumab in combination with carboplatin and etoposide)) and/or the like) from the patient's treatment plan based at least on whether the molecular subtype of the SCLC tumor sample is associated with an above-threshold likelihood of response to the treatment.

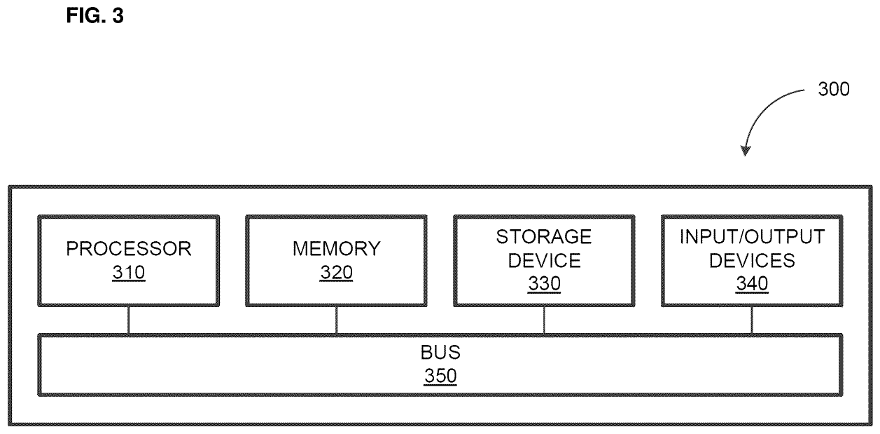

FIG. 3 depicts a block diagram illustrating an example of computing system 300, in accordance with some example embodiments. Referring to FIGS. 1 and 3, the computing system 300 may be used to implement the digital pathology platform 110, the client device 130, and/or any components therein.

As shown in FIG. 3, the computing system 300 can include a processor 310, a memory 320, a storage device 330, and input/output device 340. The processor 310, the memory 320, the storage device 330, and the input/output device 340 can be interconnected via a system bus 350. The processor 310 is capable of processing instructions for execution within the computing system 300. Such executed instructions can implement one or more components of, for example, the digital pathology platform 110, the client device 130, and/or the like. In some example embodiments, the processor 310 can be a single-threaded processor. Alternately, the processor 310 can be a multi-threaded processor. The processor 310 is capable of processing instructions stored in the memory 320 and/or on the storage device 330 to display graphical information for a user interface provided via the input/output device 340.

The memory 320 is a computer readable medium such as volatile or non-volatile that stores information within the computing system 300. The memory 320 can store data structures representing configuration object databases, for example. The storage device 330 is capable of providing persistent storage for the computing system 300. The storage device 330 can be a floppy disk device, a hard disk device, an optical disk device, or a tape device, or other suitable persistent storage means. The input/output device 340 provides input/output operations for the computing system 300. In some example embodiments, the input/output device 340 includes a keyboard and/or pointing device. In various implementations, the input/output device 340 includes a display unit for displaying graphical user interfaces.

According to some example embodiments, the input/output device 340 can provide input/output operations for a network device. For example, the input/output device 340 can include Ethernet ports or other networking ports to communicate with one or more wired and/or wireless networks (e.g., a local area network (LAN), a wide area network (WAN), the Internet).

In some example embodiments, the computing system 300 can be used to execute various interactive computer software applications that can be used for organization, analysis and/or storage of data in various formats. Alternatively, the computing system 300 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 340. The user interface can be generated and presented to a user by the computing system 300 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

Any suitable tumor sample may be assessed in the digital pathology systems disclosed herein. In some examples, the tumor sample is a formalin-fixed and paraffin-embedded (FFPE) sample, an archival sample, a fresh sample, or a frozen sample. In some examples, the tumor sample is a pre-treatment tumor sample.

The image of the tumor sample may be, e.g., an image of a slide that has been processed using a histology approach (e.g., a tissue stain (e.g., hematoxylin and eosin stain, Masson's trichome stain, a silver stain, and the like)), immunohistochemistry (IHC), immunofluorescence (IF), historadiography, and the like). Any suitable histology approach may be used.

### V. Assessment of PD-L1 Expression

The expression of PD-L1 may be assessed in a patient treated according to any of the methods, compositions for use, and uses described herein. The methods, compositions for use, and uses may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP263 antibody or the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table 2 and/or Table 3, respectively.

TABLE 2
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells covering | |
| <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells covering | |
| ≥1% to <5% of tumor area occupied by tumor cells, | |
| associated intratumoral stroma, and contiguous | |
| peri-tumoral desmoplastic stroma | |
| Presence of discernible PD-L1 staining of any | IC2 |
| intensity in tumor-infiltrating immune cells covering | |
| ≥5% to <10% of tumor area occupied by tumor | |
| cells, associated intratumoral stroma, and | |
| contiguous peri-tumoral desmoplastic stroma | |
| Presence of discernible PD-L1 staining of any | IC3 |
| intensity in tumor-infiltrating immune cells covering | |
| ≥10% of tumor area occupied by tumor cells, | |
| associated intratumoral stroma, and contiguous | |
| peri-tumoral desmoplastic stroma | |

TABLE 3
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### V. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′) 2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:
(a) a heavy chain variable region (VH) comprising the amino acid sequence:

(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,

and
(b) the light chain variable region (VL) comprising the amino acid sequence:

(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and
(b) the light chain amino acid sequence:
(SEQ ID NO: 2)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### VI. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. Any of the additional therapeutic agents described herein may also be included in a pharmaceutical composition or formulation.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20-mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14-mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VII. Articles of Manufacture or Kits

Also provided herein are articles of manufacture and kits, which may be used for classifying a patient according to any of the methods disclosed herein.

In one example, provided herein is a kit for classifying a lung cancer (e.g., SCLC, e.g., ES-SCLC or LS-SCLC, including in the 1L treatment setting) in a human patient, the kit comprising: (a) reagents for assaying mRNA in a tumor sample from the patient to provide a transcriptional profile of the patient's tumor; and (b) instructions for assigning the patient's tumor sample into one of the following four subtypes based on the transcriptional profile of the patient's tumor: neuroendocrine inflamed (NE-I), neuroendocrine NEUROD-driven (NE-N), neuroendocrine achaete-scute homolog 1 (ASCL1)-driven (NE-A), or non-neuroendocrine inflamed (nNE-I), thereby classifying the SCLC. Any suitable reagents for assaying mRNA may be included in the kit, e.g., nucleic acids, enzymes, buffers, and the like.

In another aspect, provided herein is an article of manufacture or a kit comprising a PD-1 axis binding antagonist (e.g., atezolizumab). In some instances, the article of manufacture or kit further comprises package insert comprising instructions for using the PD-1 axis binding antagonist to treat or delay progression of lung cancer (e.g., SCLC, e.g., ES-SCLC or LS-SCLC, including in the 1L treatment setting) in a patient, e.g., for a patient who has been classified according to any of the methods disclosed herein. In some instances, the article of manufacture or kit further comprises package insert comprising instructions for using the PD-1 axis binding antagonist to treat or delay progression of lung cancer (e.g., SCLC, e.g., ES-SCLC or LS-SCLC, including in the 1L treatment setting) in a patient. Any of the PD-1 axis binding antagonists and/or any additional therapeutic agents described herein may be included in the article of manufacture or kits.

In some instances, the PD-1 axis binding antagonist and/or any additional therapeutic agent are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent, e.g., carboplatin and/or etoposide). Suitable containers for the one or more agents include, for example, bottles, vials, bags, and syringes.

Any of the articles of manufacture or kits may include instructions to administer a PD-1 axis binding antagonist, or another anti-cancer therapy, to a patient in accordance with any of the methods described herein, e.g., any of the methods set forth in Section III above.

### EXAMPLES

### Example 1: Small Cell Lung Cancer Molecular Subtypes and Vulnerability to Immune Checkpoint Blockade

This Example describes an analysis of patient tumor samples from the IMpower133 (NCT02763579) trial to identify and characterize cellular subtypes of small cell lung cancer (SCLC). Following the pivotal Phase III IMpower133 study, atezolizumab (anti-PD-L1), combined with carboplatin and etoposide (CE), was the first immune checkpoint inhibitor approved for first-line treatment of extensive-stage small cell lung cancer (ES-SCLC) and is now the standard of care. However, a clearer understanding of therapeutically relevant SCLC tumor molecular subtypes is still needed.

To refine previously described SCLC subtypes, transcriptomic analyses and nonnegative matrix factorization (NMF) were conducted on 271 patient tumor samples from IMpower133. Both tumor cell-intrinsic and tumor microenvironmental features were found to define these subtypes. Two subtypes demonstrated hallmarks of immune cell infiltration but had distinct clinical outcomes. The balance of tumor-associated macrophage (TAM) to T-effector signals distinguished these two inflamed subtypes, where tumors with low TAM but high T-effector signals demonstrated longer overall survival with PD-L1 blockade combined with CE versus CE alone. These data define distinct inflamed subtypes in SCLC that benefit from immunomodulation therapy.

### A. Study Design and Rationale

Until recently, the standard first-line treatment for patients with ES-SCLC was carboplatin or cisplatin and etoposide (EP) chemotherapy (Byers and Rudin. Cancer. 121:664-672 (2015); Farago and Keane. Transl Lung Cancer Res. 7:69-79 (2018)). The addition of immunotherapy to traditional chemotherapy has improved overall survival (OS) and progression-free survival (PFS) in patients with ES-SCLC, as evidenced by results from global randomized Phase III clinical trials, such as IMpower133 (Horn et al. N Engl J Med. 379:2220-2229 (2018); Liu et al. J Clin Oncol. 39:619-630 (2021)) and CASPIAN (Paz-Ares et al. Lancet. 394:1929-1939 (2019)). Based on results from the IMpower133 trial, atezolizumab in combination with carboplatin and etoposide (CE) was the first immune checkpoint inhibitor approved for first-line treatment of ES-SCLC (Liu et al. J Clin Oncol. 39:619-630 (2021)), shifting the treatment paradigm in this disease area.

Despite immunotherapy being added to platinum-based frontline chemotherapy, improvements in OS and PFS for patients with ES-SCLC have been modest (Horn et al. N Engl J Med. 379:2220-2229 (2018); Liu et al. J Clin Oncol. 39:619-630 (2021); Reck et al. J Thorac Oncol. 17:1122-1129 (2022)). In general, SCLC tumors are immunological deserts, have low major histocompatibility complex (MHC) expression, and the tumor cells have low PD-L1 expression, potentially contributing to the modest improvement observed with immunotherapy plus platinum chemotherapy (Gay et al. Cancer Cell. 39:346-360.e7 (2021); Liu et al. J Clin Oncol. 39:619-630 (2021); Mansfield et al. Ann Oncol. 31:310-317 (2020); Thomas et al. J Thorac Oncol. 14:1447-1457 (2019); Zimmermann et al. Am Soc Clin Oncol Educ Book. 38:682-695 (2018)). Therefore, a better understanding of the molecular features of SCLC that are associated with response to targeted and immunotherapies is needed.

In the current study, patient tumor samples were used from the IMpower133 trial, which contained a larger data set than previous studies, to identify and characterize cellular subtypes of SCLC using nonnegative matrix factorization (NMF) to further refine previously described subtypes (Gay et al. Cancer Cell. 39:346-360.e7 (2021); Liu et al. J Clin Oncol. 39:619-630 (2021); Mansfield et al. Ann Oncol. 31:310-317 (2020); Zhang et al. Transl Lung Cancer Res. 7:32-49 (2018)).

### B. Materials and Methods

i. Patients

The design of the randomized, double-blind IMpower133 trial has been reported previously (Horn et al. N Engl J Med. 379:2220-2229 (2018); Liu et al. J Clin Oncol. 39:619-630 (2021); Mansfield et al. Ann Oncol. 31:310-317 (2020)). Patients with chemotherapy-naive ES-SCLC were stratified by sex (male versus female), Eastern Cooperative Oncology Group (ECOG) performance status (PS; 0 versus 1), and presence of brain metastases (yes versus no).

Patients were randomly assigned 1:1 to receive four 21-day cycles of CE with either intravenous (IV) atezolizumab 1200 mg or IV placebo on day 1 of each cycle (induction phase), followed by IV atezolizumab or placebo according to randomized assignment (maintenance phase), until unacceptable toxicity or disease progression. Patients could continue treatment after progression per Response Evaluation Criteria In Solid Tumors 1.1 (RECIST 1.1) if there was evidence of clinical benefit. Prophylactic cranial irradiation (PCI) was permitted during the maintenance phase. The co-primary endpoints were OS and investigator-assessed PFS in the intention-to-treat population.

In the current study, tumors from 271/403 (67%) patients were transcriptionally profiled by RNA sequencing (RNA-seq) and tumor mutational burden was profiled by DNA whole exome sequencing (WES).

ii. Sample Handling

Clinical samples were received in the lab immediately after extraction (median delivery time±SEM, 0.75±0.72 hours) and processed rapidly (median±SEM processing time from delivery until 10× protocol started, 1.75±0.27 hours) to ensure high sample viability and quality for RNA-seq.

iii. RNA-seq Sample Collection and Sequencing

Using hematoxylin and eosin (H&E) as a guide, formalin-fixed paraffin-embedded tissue (FFPET) was macro-dissected for the tumor area. RNA was extracted using the High Pure FFPE RNA Isolation Kit (Roche) and assessed by QUBIT™ (Thermo Fisher Scientific) and Agilent Bioanalyzer for quantity and quality. First strand cDNA synthesis was primed from total RNA using random primers, followed by the generation of second strand cDNA with dUTP in place of dTTP in the master mix to facilitate preservation of strand information. Libraries were enriched for the mRNA fraction by positive selection using a cocktail of biotinylated oligos corresponding to coding regions of the genome. Libraries were sequenced using the Illumina sequencing method.

iv. RNA-seq Data Generation and Processing

TruSeq technology (Illumina) was used to generate whole-transcriptome profiles. To remove ribosomal reads, RNA-seq reads were first aligned to ribosomal RNA sequences. GSNAP version 2013 Oct. 10 was used to align the remaining reads to the human reference genome (NCBI Build 38), allowing a maximum of two mismatches per 75 base sequences (parameters: ‘-M 2-n 10-B 2-i 1-N 1-w 200000-E 1-pairmax-rna=200000-clip-overlap) (Wu and Nacu. Bioinformatics. 26 (7): 873-881 (2010); Wu et al. Methods Mol Biol. 1418:283-334 (2016)). To quantify gene expression levels, the number of reads mapped to the exons of each RefSeq gene was calculated using the functionality provided by the R/Bioconductor package GenomicAlignments. Raw counts were adjusted for gene length using transcript-per-million (TPM) normalization, and subsequently log 2-transformed.

v. Tumor Whole-Exome Sequencing and Variant Calling

Whole-exome libraries were prepared from tumor FFPE DNA and matched germline DNA using the Agilent SureSelect v6 and sequenced at 2×150 bp. Fastq file quality checks were performed with FastQC (v.0.11.9). Fastqs were pre-processed and aligned to hg38 using Picard (v2.18), Burrows-Wheeler Aligner (v0.7.15-r1140) (Li and Durbin. Bioinformatics. 25:1754-1760 (2009)), and Genome Analysis Toolkit v4.1.4.1 (DePristo et al. Nat Genet. 43:491-498 (2011)).

Tumor/normal pair confirmation was provided by NGSCheckmate (Lee et al. Nucleic Acids Res. 45: e103 (2017)). Variant calling was done by Mutect2 (Cibulskis et al. Nat Biotechnol. 31:213-219 (2013)), LoFreq2 (Wilm et al. Nucleic Acids Res. 40:11189-11201 (2012)), and Strelka (Saunders et al. Bioinformatics. 28:1811-1817 (2012)) and annotated using Ensembl Variant Effect Predictor (VEP) (McLaren et al. Genome Biol. 17:122 (2016)). Nonsynonymous variants with a VEP score of moderate or high were only reported if identified by 2 of 3 variant callers.

vi. Non-negative Matrix Factorization (NMF)

Unsupervised machine learning approach based on consensus non-negative matrix factorization (cNMF) was applied to normalized RNA-seq data to identify transcriptomic-based subtypes. This type of clustering is based on the dimensional reduction methodology of NMF which reduces the expression data from thousands of genes to a few metagenes (CRAN. R package version 0.22.0) (Brunet et al. Proc Natl Acad Sci USA. 101:4164-4169 (2004)) combined with the consensus clustering to test stability of iterative NMF runs. This method computes multiple k-factor factorization decompositions of the expression matrix and evaluates the stability of the solutions using a cophenetic coefficient. Median Absolute Deviation (MAD) analysis was used to select 5829 genes (top 10%) with the highest variability across 271 tumors. Then, consensus NMF clustering was applied, testing k=2 to k=8, and identifying k=4 as the most robust subtypes using the cophenetic correlation.

vii. Validation of NMF Clustering in IMpower133

To validate molecular subtypes derived in IMpower133, the random forest machine learning algorithm (R package random-Forest) was used to derive a classifier and then predict the NMF clusters in an independent data set (IMpower133). A random forest classifier involves learning a large number of binary decision trees from random subsets of a training set. These trees in the classifier can then be used in a prediction algorithm to identify the similarity of a given sample to a given class in the training set. Before learning the random forest classifier, the data was preprocessed to generate the training set. To ensure accurate prediction of all four NMF classes we down-sampled by randomly removing observation from the majority classes to prevent its signal from dominating the learning algorithm. Next, we normalized the gene expression values in each patient by ranking the gene expression values to ensure that the test and training set were on the same scale. To predict the NMF subtypes derived in Mpower133, we applied the classifier to each patient sample in the test cohort. In some examples, the random forest classifier includes the genes set forth in Table 1.

viii. Quantitative Set Analysis for Gene Expression (QuSAGE)

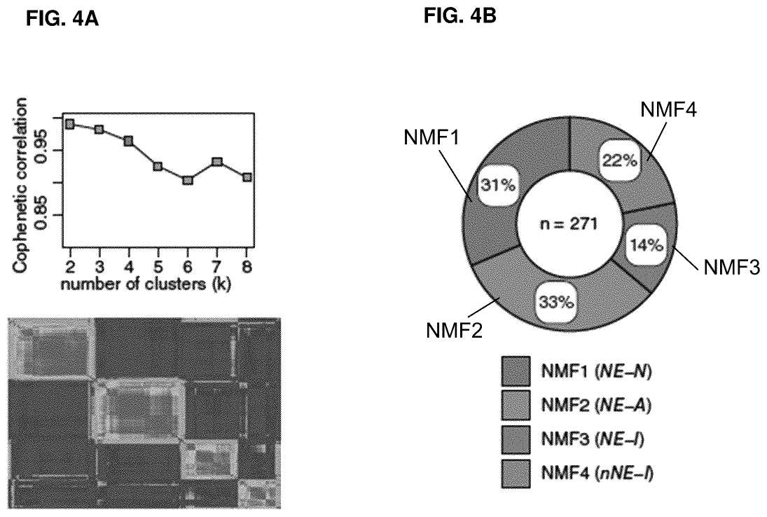

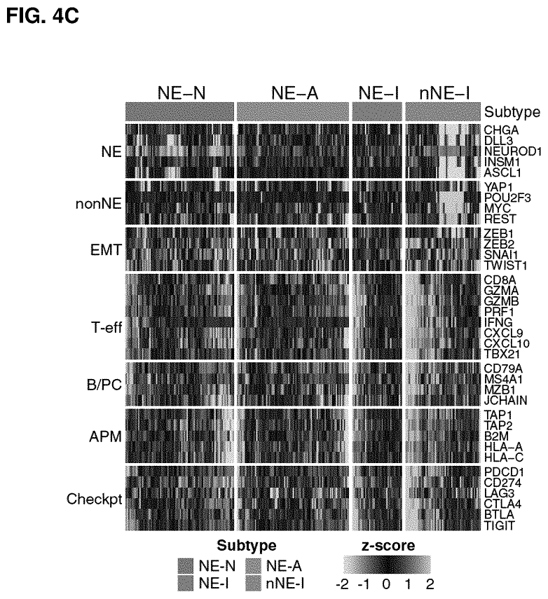

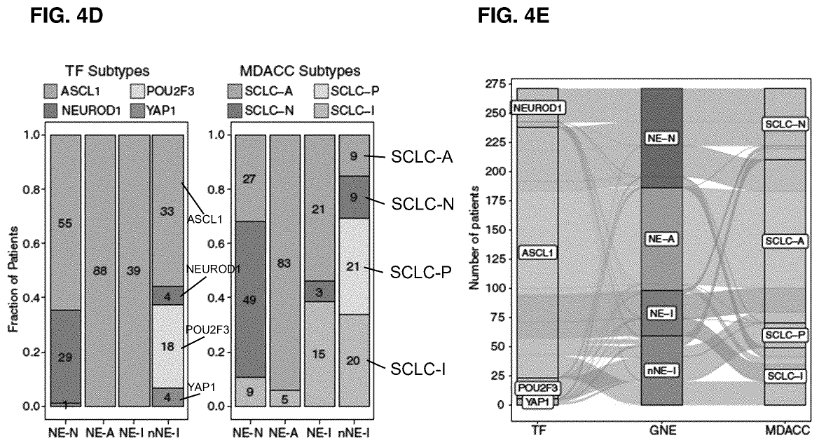

In order to understand the biological pathways underlying NMF clustering, QuSAGE analysis (R/Bionconductor qusage v2.18.0) was performed to compare each cluster to all others, leveraging MSigDb hallmark gene sets to identify enriched pathways within each cluster. Enrichment scores were represented as a heatmap (FIG. 4A).

### ix Quantification and Statistical Analysis

Rv3.6.1 was used for all analyses. The two-sided Mann-Whitney test (R function Wilcox.test) for two groups and the Kruskal-Wallis test (R function Kruskal.test) for more than two groups were used for all comparisons for continuous variables, unless otherwise stated. Dunn's post-hoc test was applied with Benjamini-Hochberg multiple testing correction for pairwise comparisons. Pearson's Chi-squared test with continuity correction was used (R function chisq.test) for categorical variables. FDR-adjusted P-values are reported. * P<0.05; ** P<0.01; *** P<0.001, unless otherwise stated. Survival analyses were conducted using Cox-proportional hazard models using the R survival package (v3.1.7). Log-rank P-values were reported for survival analyses including more than two groups.

The horizontal line represents the median in all box plots. The lower and upper hinges in all box plots correspond to the first and third quartiles. The upper whisker extends from the hinge to the largest value no further than 1.5*IQR from the hinge (where IQR is the interquartile range, or distance between the first and third quartiles). The lower whisker extends from the hinge to the smallest value at most 1.5*IQR of the hinge.

### C. Results

i. Refined SCLC Molecular Subtypes Derived from IMpower133 Transcriptomics

To unbiasedly identify SCLC subtypes, a non-negative matrix factorization (NMF) approach was employed (Skoulidis et al. Cancer Discov. 5:860-877 (2015)), using 271 patient samples from the IMpower133 trial (Horn et al. N Engl J Med. 379:2220-2229 (2018); Mansfield et al. Ann Oncol. 31:310-317 (2020)). A cophenetic correlation was performed for an increasing number of clusters for NMF (top panel, FIG. 4A), which determined that k=4 was the optimal number of clusters based on the drop-off of the cophenetic correlation from k=4 to k=8 (FIG. 4A). Distribution of the four NMF-identified patient clusters showed that NMF1 and NMF2 had the most patients, with 31% (n=84/271) and 33% (n=89/271), respectively. NMF3 and NMF4 had smaller percentages of patients, with 14% (n=38/271) and 22% (n=60/271), respectively (FIG. 4B).

Based on the distribution of known gene expression pathways, the NMF-identified clusters were broadly characterized into neuroendocrine NEUROD1-driven (NE-N; NMF1), neuroendocrine ASCL1-driven (NE-A; NMF2), neuroendocrine inflamed (NE-I; NMF3), and nonneuroendocrine inflamed (nNE-I; NMF4) (FIG. 4C). Prior classification schema identified one inflamed subgroup (YAP1 or SCLC-I) (Gay et al. Cancer Cell. 39:346-360.e7 (2021); Rudin et al. Nat Rev Cancer. 19:289-297 (2019)), whereas in this study, two distinct clusters were identified with the hallmarks of lymphocyte inflammation. Both a NE and a nNE subgroup were found to be enriched for T cells, B/plasma cells, checkpoint molecules, and antigen presentation machinery (APM) (FIG. 4C).

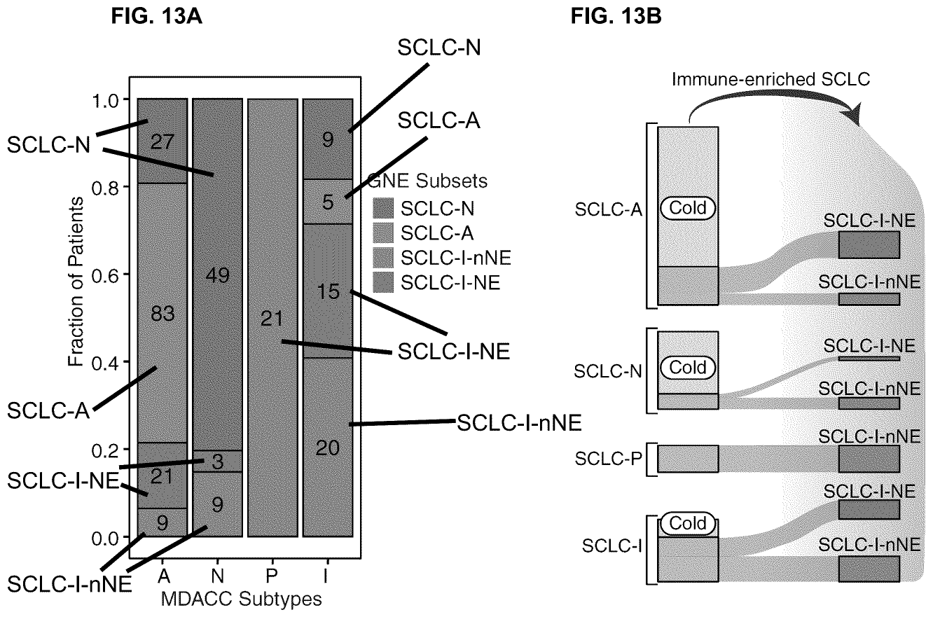

The distribution of prior subtyping approaches was compared using the single transcription factors (TF Subtypes) (Rudin et al. Nat Rev Cancer. 19:289-297 (2019)) or the NMF-based subtyping approach (MD Anderson Cancer Center (MDACC) Subtypes) (Gay et al. Cancer Cell. 39:346-360.e7 (2021)) defined for limited-stage (LS)-SCLC tumors from a smaller public dataset (George et al. Nature. 524:47-53 (2015)) (FIGS. 4D and 13A). The NE-N subtype contained almost all previously identified NEUROD1 tumors by either approach, the NE-A and NE-I subtypes were both classified as ASCL1 by the TF approach, the nNE-I subtype contained the POU2F3 tumors using either approach and the YAP1 subtype by the TF approach, while the newly identified SCLC-I tumors were split between the NE-I and nNE-I subtypes (FIG. 4E). Few tumors were classified as a YAP1 subtype by the TF approach; YAP1 expression was seen across subtypes and was associated with EMT-related gene programs, which confirmed prior studies that suggested it does not uniquely define a subtype (Gay et al. Cancer Cell. 39:346-360.e7 (2021); Shue et al. Nat Commun. 13:2690 (2022); Wu et al. Sci. Adv. 7, eabg1850 (2021)). These data suggest that SCLC molecular subtypes can be distinguished by both transcription factor drivers and immune infiltration status. Previously reported subtypes can be split into immune cold and immune enriched SCLC, where immune-enriched SCLC can be further delineated into SCLC-I-NE and SCLC-I-nNE based on cell-intrinsic features (FIG. 13B). In total, these data recapitulate and extend prior subtyping classifications by uncovering immune heterogeneity within subtypes.

ii. Cell-Intrinsic Determinants of SCLC Molecular Subtypes

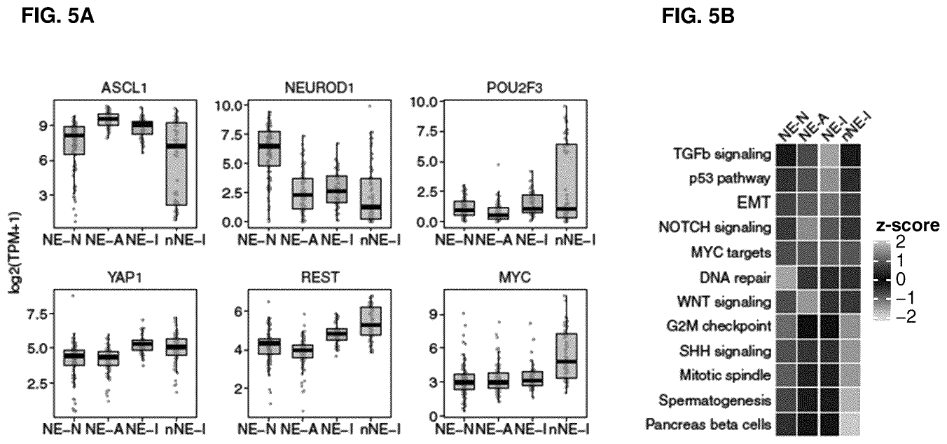

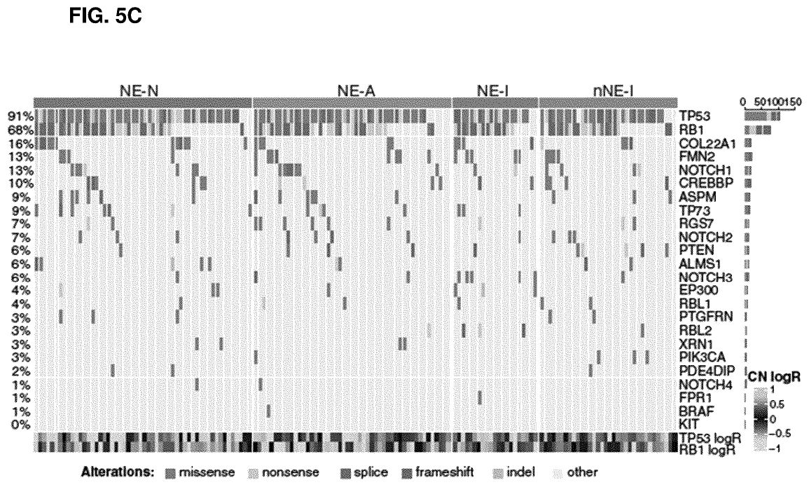

To begin identifying molecular distinctions between the four NMF subtypes, the transcription factor expression profiles of the subtypes were examined. The NE-A and NE-I subtypes had the highest ASCL1 expression, and NE-N had uniquely high NEUROD1 expression (FIG. 5A). Prior classification of SCLC-I tumors noted low ASCL1 expression, suggesting a more neutral subtype, while our analyses suggest only NMF4 has low ASCL1 expression (FIG. 5A). POU2F3 was only expressed in a subset of nNE-I. RE1 Silencing Transcription Factor (REST) and MYC were uniquely elevated in most nNE-I tumors, suggesting differential nNE drivers within the nNE-I subtype. YAP1 was similarly elevated in both inflamed subtypes, consistent with prior literature (Rudin et al. Nat Rev Cancer. 19:289-297 (2019)) (FIG. 5A).

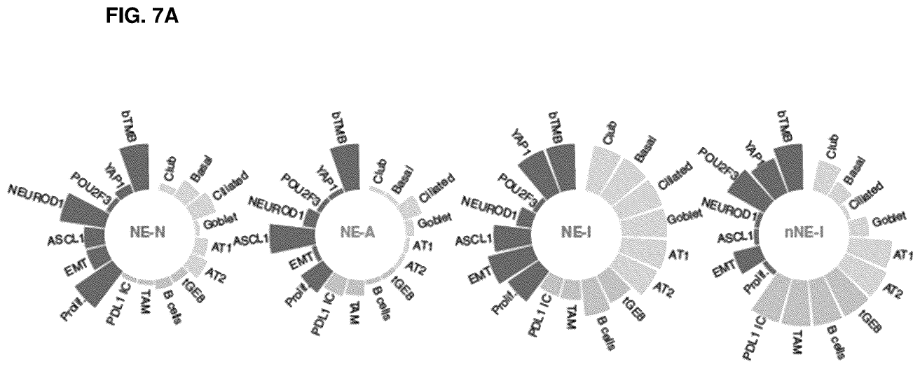

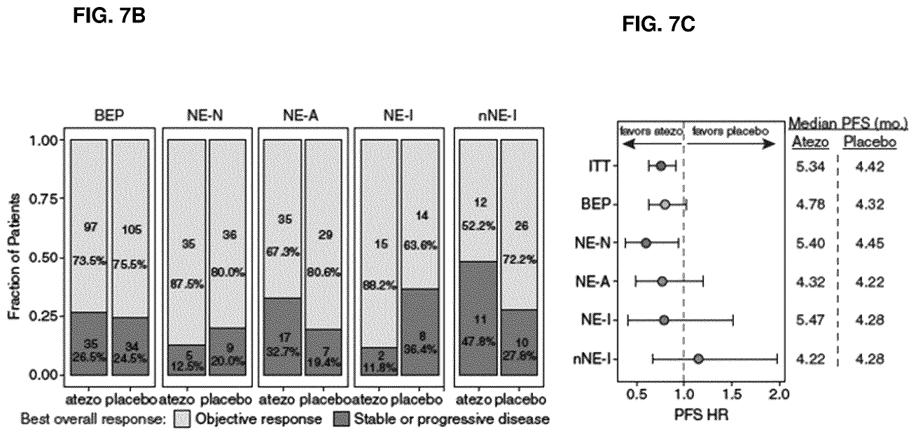

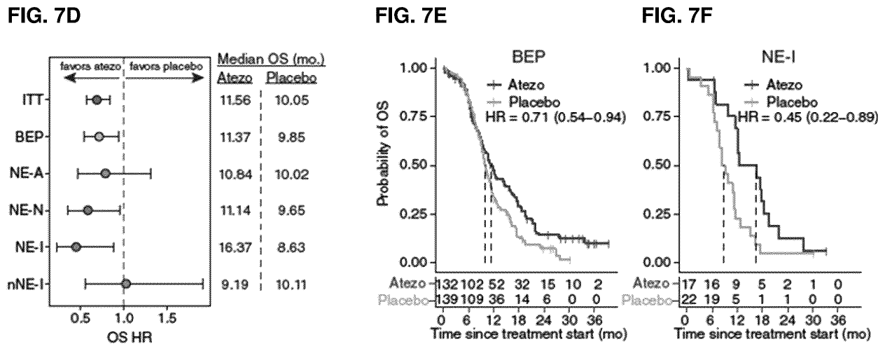

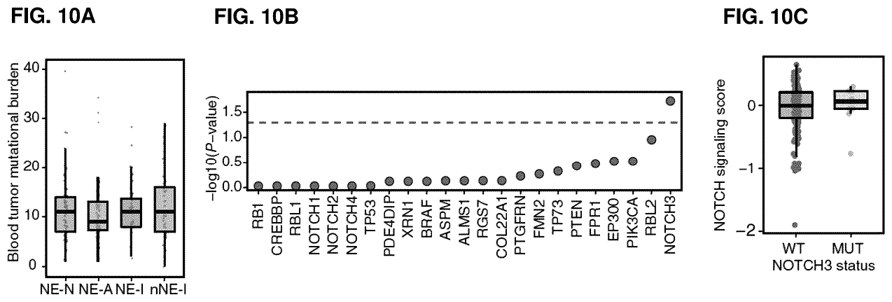

By examining differentially expressed hallmark gene sets (Liberzon et al. Cell Syst. 1:417-425 (2015)) in pairwise comparisons between the gene sets, the NE-N subtype showed evidence of enhanced proliferation and associated DNA repair, while the inflamed subtypes (NE-I and nNE-I) appeared to be the most mesenchymal (FIG. 5B). As expected, based on MYC expression, the nNE-I had elevated MYC target gene expression (FIG. 5B). Previously identified commonly mutated genes (George et al. Nature. 524:47-53 (2015)) were not differentially mutated in each subtype (FIGS. 5B and 7B). Copy number analyses of TP53 and RB1 showed likely loss in cases where a nonsynonymous mutation did not appear, which suggested inactivation (FIGS. 5B and 7B). Therefore, genotyping did not inform the molecular subtypes. Blood tumor mutation burden (bTMB) was similar across subtypes (FIG. 10A), and NOTCH3 mutations that were somewhat enriched in NE-I tumors did not confer differentially hallmark NOTCH signaling (FIGS. 10B and 10C).

iii. Tumor Microenvironmental Features of SCLC Molecular Subtypes

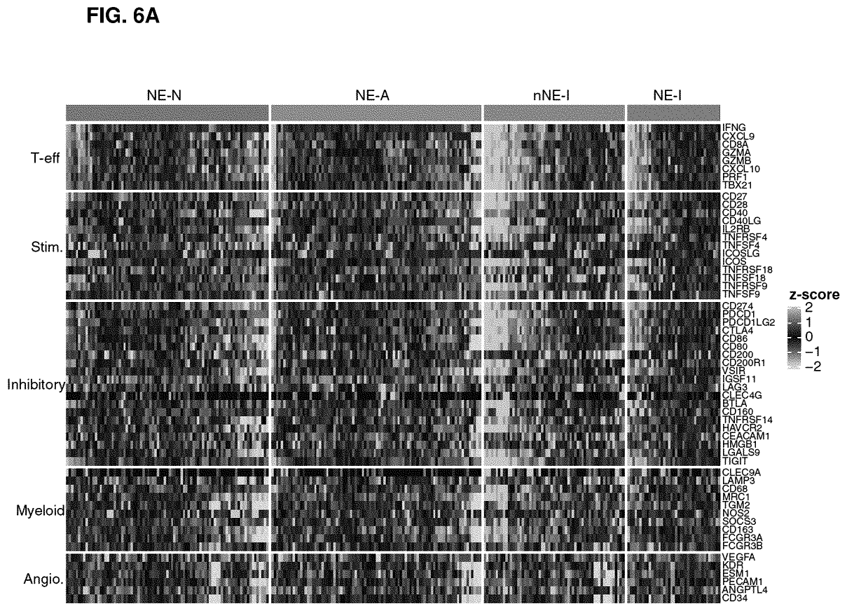

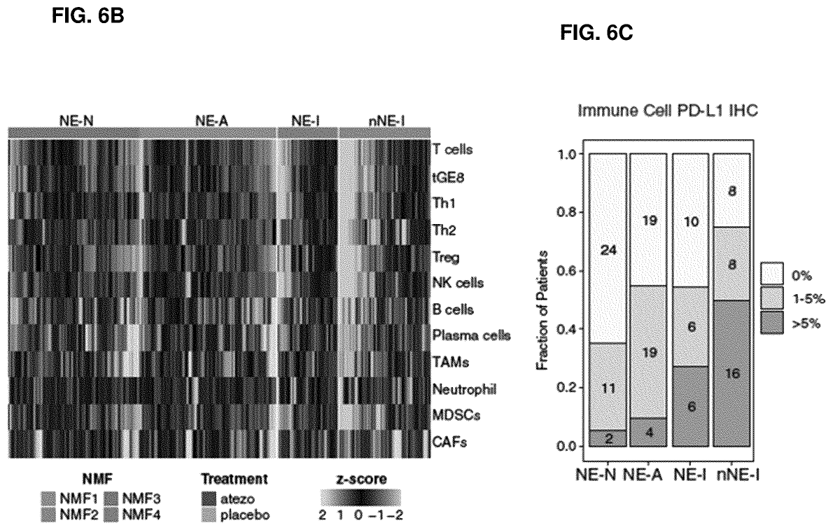

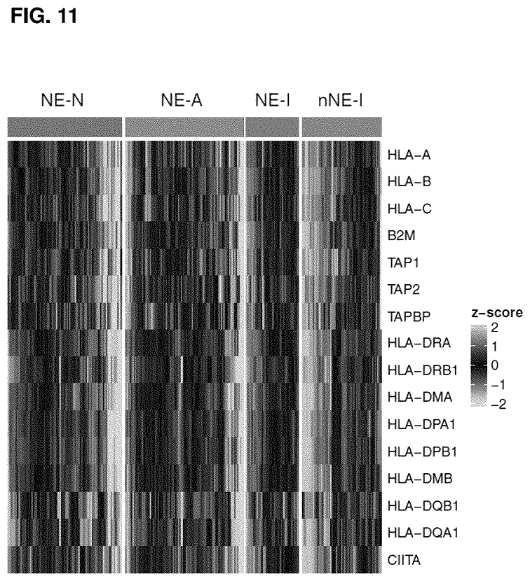

To better define the tumor microenvironment (TME) features of the NMF subtypes, the expression of genes related to immune cells and angiogenesis was examined for the subtypes. TME-related gene signature expression was also examined for the subtypes. Individual gene sets (FIG. 6A) and gene sets corresponding to TME cell types (FIG. 6B) demonstrated that compared with the non-inflamed subtypes (NE-N and NE-A), the inflamed subtypes (NE-I and nNE-I) were similarly elevated for T-effector cells, immune stimulatory molecules, immune inhibitory checkpoints, lymphocytes, total myeloid cells, endothelial cells, and cancer-associated fibroblasts. Therefore, NE-N and NE-A can be broadly characterized as immune cold SCLC and NE-I and nNE-I as immune infiltrated SCLC. Similarly, immune cell PD-L1 expression was elevated in the two inflamed subtypes (NE-I and nNE-I) compared with the non-inflamed subtypes (FIG. 6C). A heatmap showed that APM genes were similarly elevated in nNE-I and NE-I tumors (FIG. 11). In total, we identified four subsets defined by their cell-intrinsic and -extrinsic features that both recapitulate and suggest heterogeneity and potentially therapeutically relevant within previously reported SCLC features. Therefore, it was hypothesized that both NE-I and nNE-I would derive enhanced clinical benefit from atezolizumab plus CE versus placebo plus CE.

iv. Clinical Outcomes of Atezolizumab Plus CE Versus Placebo Plus CE in all Subtypes

Next, analyses were performed to determine whether there was a correlation between different NMF subtypes and clinical outcomes. A summary of features associated with each newly defined NMF subtype showed that the level of T-effector cells (tGE8) and B cells was higher in the inflamed subtypes (NE-I and nNE-I) compared with the non-inflamed subtypes (NE-N and NE-A) (FIG. 7A). Furthermore, when comparing the inflamed subtypes, the nNE-I subtype had higher levels of PD-L1 expressing ICs and TAMs compared with the NE-I subtype (FIG. 7A).

The distribution of responders (CR/PR) and non-responders (SD/PD) by best overall response in the IMpower133 RNA-seq biomarker evaluable population (BEP) was similar to that of the overall study population (Horn et al. N Engl J Med. 379:2220-2229 (2018)) (FIG. 7B). The nNE-I subtype had relatively fewer responders in the atezolizumab arm, while the NE-I subtype had a somewhat increased response rate in the atezolizumab arm compared with a reduced response rate in the placebo arm (FIG. 7B). PFS distribution in the intent-to-treat population, BEP, and each NMF subtype was relatively similar.

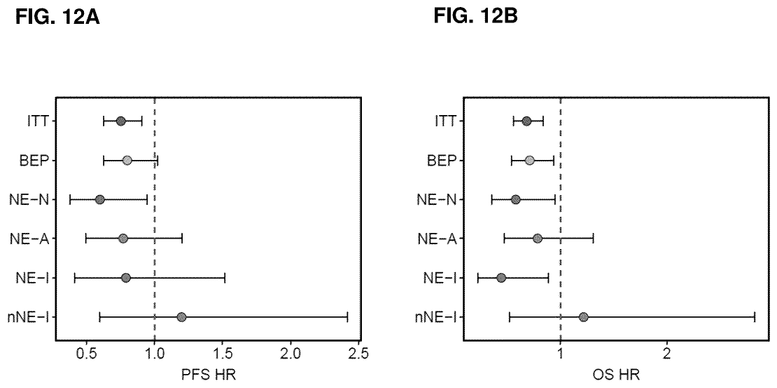

Of note, patients in the NE-I subtype treated with atezolizumab had the longest median PFS (mPFS, 5.47 months), while patients in the nNE-I atezolizumab treated subtype demonstrated the shortest (mPFS, 4.22 months) (FIG. 7C). Comparing the OS distribution within the atezolizumab arm of the different NMF subtypes demonstrated that the NE-A (median OS (mOS), 10.84 months) and NE-N (mOS, 11.14 months) subtypes exhibited similar results to the intent-to-treat population (mOS, 11.56 months) and BEP (mOS, 11.37 months) (FIG. 7D). In contrast to the non-inflamed subtypes, the inflamed subtypes, NE-I (mOS, 16.37 months) and nNE-I (mOS, 9.19 months), had markedly distinct outcomes from the other groups. The NE-I subtype had a near doubling of mOS with atezolizumab plus CE compared with placebo plus CE, while the nNE-I subtypes demonstrated no benefit despite hallmarks of lymphocyte inflammation and PD-L1 positivity (FIG. 7D). The Kaplan-Meier curves for the BEP (FIG. 7E) and NE-I group (FIG. 7F) further demonstrated the longer OS in the atezolizumab arm of the NE-I patients compared with the atezolizumab arm of the BEP. Removal of the SCLC-P tumors from the nNE-I subtype did not change the outcome associations observed (FIGS. 12A and 12B).

v. Cell-of-Origin and Myeloid Infiltration Distinguish Inflamed Subtypes

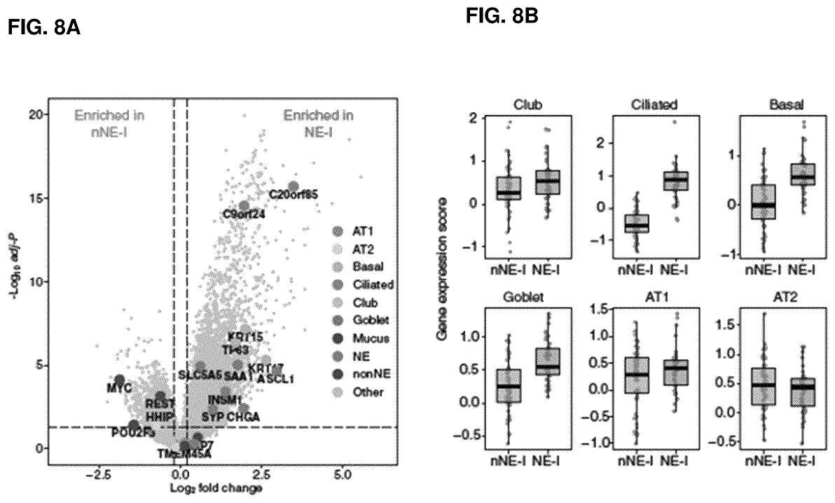

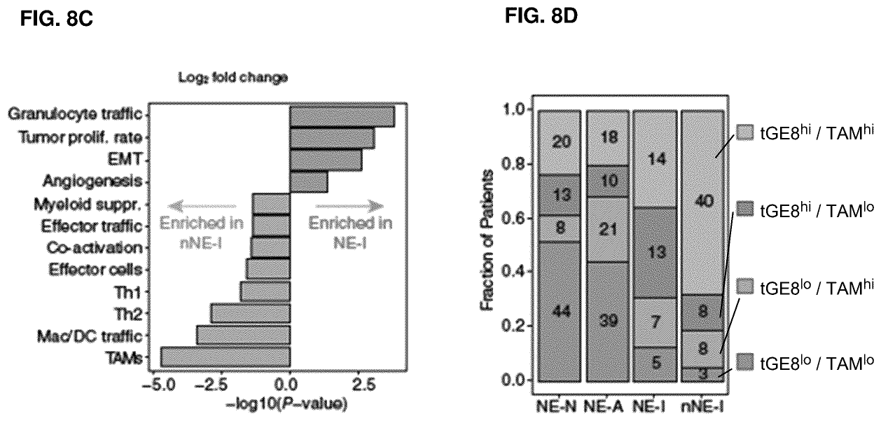

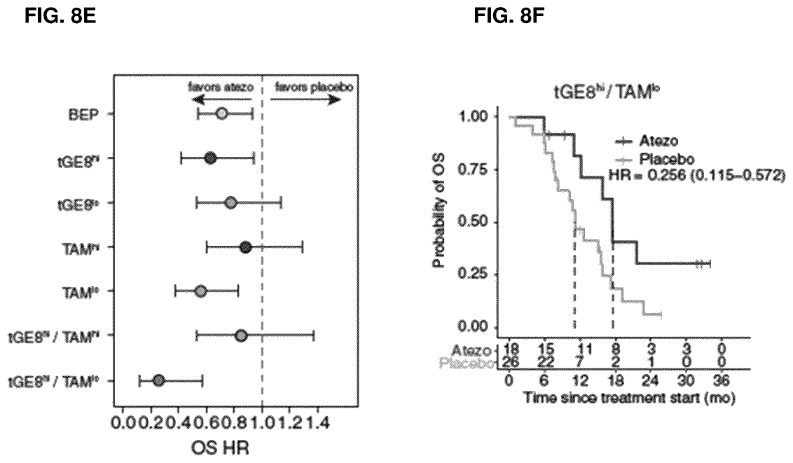

To distinguish features that may regulate the differential clinical outcomes, differential gene expression analysis was performed to compare the NE-I and nNE-I tumors (FIGS. 8A and 8B). Signals of cell-intrinsic features were found, such as lung cell lineages that were differentially expressed. For example, in addition to NE and nNE features, ciliated cell, basal cell, and goblet cell-related genes were elevated in the NE-I tumors compared with the nNE tumors (FIGS. 8A and 8B). This may be related to the cell of origin of these tumors or location of the tumors in the lung. There was no indication from pathologic examination if either subtype was enriched in tumors originating from distinct sites in the lung that would be enriched in different normal lung cells (e.g., more centrally located).

Gene sets were also examined that corresponded to the cell types that may make up the SCLC TME and be differentially associated with each tumor. Interestingly, we found that while levels of lymphocytes were largely similar, signals of TAMs, which are immune suppressive macrophages, and the chemokines that may recruit them, were highly enriched in nNE-I tumors compared with NE-I tumors (FIG. 8C). To further characterize these tumors, the tumors were delineated as T-effector cell (T-eff) high/low (tGE8) and TAM high/low based on the median cohort-wide expression for these gene signatures. Compared with the non-inflamed subtypes (NE-N and NE-A), the inflamed NE-I and nNE-I subtypes were both enriched for T-eff high tumors (≈70%), but the balance of T-eff/TAM signals between the inflamed subtypes was markedly different (FIG. 8D). nNE-I tumors that were T-eff high were almost exclusively also TAM high, while those that were NE-I and T-eff high were balanced between TAM high and TAM low (FIG. 8D).

To determine whether these signals might explain the differential clinical outcomes of atezolizumab plus CE versus placebo plus CE in the inflamed subtypes, the outcomes in the T-eff/TAM tumors were examined. Strikingly, T-eff high/TAM low tumors showed some additional OS benefit of atezolizumab plus CE compared with all other groups (FIG. 8E). Furthermore, within T-eff high/TAM low tumors, which were enriched in the NE-I subtype, atezolizumab plus CE treated tumors showed markedly longer OS than placebo plus CE tumors (HR=0.26 (95% CI: 0.12-0.57)) (FIG. 8F).

vi. REST Expression is Associated with Enhanced TAM Infiltration

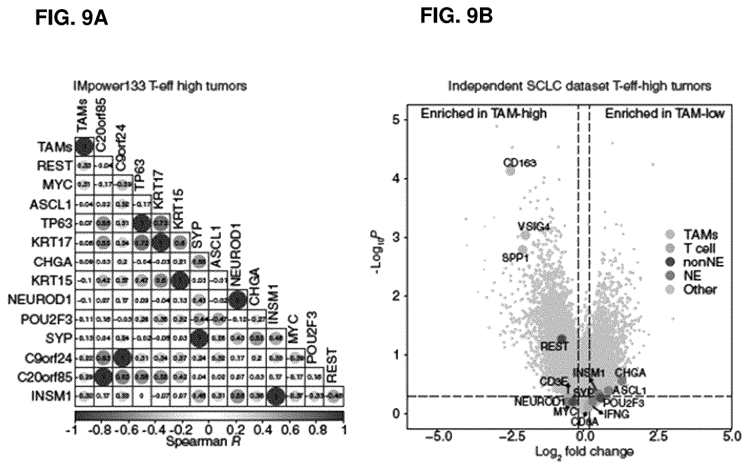

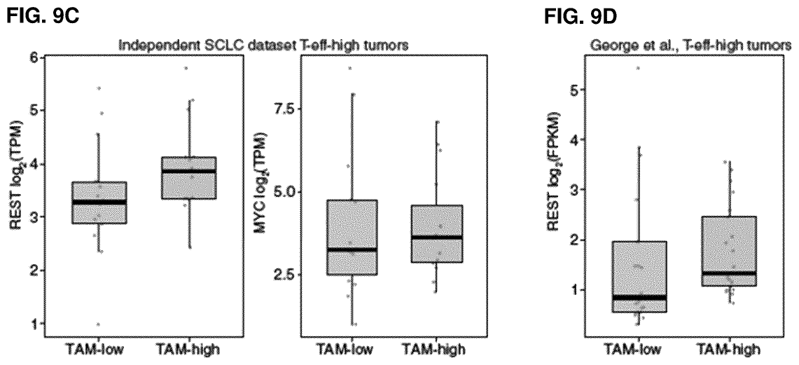

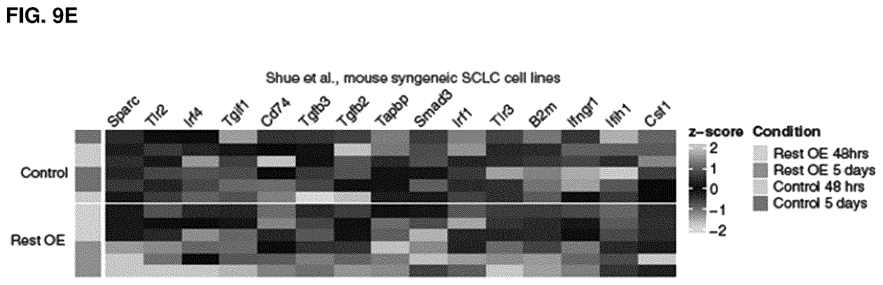

To understand which cell-intrinsic signals delineate the refined molecular subtypes that may regulate TAM infiltration in inflamed tumors, the features that best correlated with TAM signals in T-eff high tumors in IMpower133 were assessed. REST and MYC expression were found to be positively correlated with myeloid infiltration (FIG. 9A). To further characterize this in independent data an additional 58 LS/ES-SCLC samples were procured and analyzed by RNA-seq to compare differentially expressed genes within the T-eff high tumors in this cohort between TAM high and TAM low tumors (FIGS. 9B and 9C). Relative T cell signals were not distinct in this subgroup (FIG. 9B), but REST was prominently differentially expressed, while MYC was not (FIG. 9C). In publicly available data (George et al. Nature. 524:47-53 (2015)), a similar pattern was observed, which was that REST expression was elevated in T-eff high/TAM high compared with T-eff high/TAM low (FIG. 9D).

In total, these data suggest that REST expression in T-eff inflamed tumors might regulate TAM infiltration. To test this hypothesis, available data was analyzed from mouse syngeneic SCLC cell lines derived from trp53/Rb1/Rbl2 (TKO) GEMMs where REST was overexpressed in culture for 48 hours or 5 days (Shue et al. Nat Commun. 13:2690 (2022)) (FIG. 9E). Here it was found that REST overexpression resulted in enhanced APM/pro-inflammatory signals (IFNGr1, B2m, Irf1, CD74, Tapbp); however, REST also concomitantly increased signals that may result in immunosuppression, such as TGFβ signaling (Tgfb2, Tgfb3, Smad3) and myeloid chemokines (Csf1) (FIG. 9E). In summary, these data suggest a role for REST in recruiting immune-suppressive myeloid cells that may dampen T-effector activity and may explain the relatively poorer outcomes of patients with SCLC-I-nNE tumors.

vii. Discussion

In this study, four subtypes were identified that are defined by both cell-intrinsic and microenvironmental features. The subtypes were broadly characterized into SCLC-N-enriched, neuroendocrine NEUROD1-driven (NE-N); SCLC-A-enriched neuroendocrine ASCL1-driven (NE-A); SCLC-I and SCLC-A-enriched, neuroendocrine inflamed (NE-I); and SCLC-P and SCLC-I enriched, non-neuroendocrine inflamed (nNE-I) (FIGS. 4C and 7A). Prior classification schema had one inflamed subgroup (SCLC-I), and tentatively identified a second inflamed subgroup SCLC-P (characterized by higher POU2F3 levels) (Gay et al. Cancer Cell. 39:346-360.e7 (2021); Huang et al. Genes Dev. 32, 915-928 (2018); Rudin et al. Nat Rev Cancer. 19:289-297 (2019)). Our analysis identifies two distinct clusters with inflamed hallmarks. The results showed both a NE and nNE group with enrichment for T cells, B/plasma cells, antigen presentation machinery (APM), and immune cell PD-L1 positivity (i.e., the neuroendocrine NE-I subtype and the non-neuroendocrine nNE-I subtype, respectively) (FIG. 4C). The nNE-I subtype expressed higher levels of non-neuronal transcription factors, such as POU2F3, while the NE-I subtype expressed the transcription factor ASCL1 and had the most similarity with the previously described SCLC-I subtype (Gay et al. Cancer Cell. 39:346-360.e7 (2021)). Regarding clinical outcomes, it was found that while the NE-A and NE-N subtypes show similar atezolizumab plus CE benefit compared to placebo, the inflamed subtypes had markedly distinct outcomes. The NE-I subtype showed a near doubling of median OS with atezolizumab plus CE compared to placebo plus CE (OS HR, 0.45 (0.22-0.89)), while the nNE-I subtype showed no benefit despite hallmarks of lymphocyte inflammation and PD-L1 positivity (OS HR, 1.02 (0.55-1.91)). The balance of T-effector to TAM signals distinguished these two inflamed subtypes, where tumors with high T-effector, but low TAM signals demonstrated markedly longer overall survival with the addition of PD-L1 blockade to CE compared to CE alone (OS HR, 0.26 (0.12-0.57)). Mechanistically, analyses in independent datasets suggested that cell-intrinsic features may govern enhanced TAM infiltration.

Clinically, SCLC subtype classification is significant, as each subtype may be uniquely susceptible to different investigational therapies. It was observed that DLL3 protein is more highly expressed in the neuroendocrine SCLC-A tumor subtype, which is most similar to NE-A and NE-N, and it is virtually unexpressed in SCLC-I and SCLC-P tumors, which are similar to the inflamed subtypes, NE-I and nNE-I (Gay et al. Cancer Cell. 39:346-360.e7 (2021)). Therefore, the neuroendocrine subtypes NE-A, which is exclusively an SCLC-A (ASCL1 positive) subtype, and NE-N, which was a mix of the SCLC-A and SCLC-N(NEUROD1 positive) subtypes, may be good candidates for DNA damage response (DDR) targeting agents, such as delta-like ligand 3 (DLL3) antibody-drug conjugate (ADC) therapy (Owen et al. J Hematol Oncol. 12:61 (2019)). DLL3-targeted Rova-T is an ADC consisting of a humanized IgG1 monoclonal antibody against DLL3, a pyrrolobenzodiazepine (PDB) dimer toxin, and a protease-cleavable linker that covalently binds the antibody to the toxin. When the Rova-T ADC binds to DLL3, it is internalized to lysosomes, the linker is broken, toxins are released and cause DNA damage, leading to apoptosis (Owen et al. J Hematol Oncol. 12:61 (2019)). BiTE is another DLL3-based therapeutic strategy that may have potential for the NE-A and NE-N subtypes, whereby the AMG 757 antibody construct transiently connects DLL3-positive cells to CD3-positive T cells, resulting in serial lysis of tumor cells and the concomitant proliferation of T cells (Giffin et al. Clin Cancer Res. 27:1526-1537 (2021)).

The NE-I and nNE-I subtypes were both inflamed, which suggested that patients in these subtypes would have better OS than those in the NE-A and NE-N subtypes, but this was not the case. When looking at the expression of TAMs and T-effector cells, the NE-I subtype group, which had the highest number of patients with low TAM and high T-eff levels, had the longest mOS (16.37 months) with atezolizumab treatment compared with the other subgroups. The nNE-I subtype group contained the most patients with high TAM and high T-eff, and had the shortest mOS (9.19 months) with atezolizumab treatment compared with the other subgroups. The NE-N subtype, which had the most patients with low T-eff and low TAM, had a longer mOS (11.14 months) with atezolizumab treatment compared with the inflamed nNE-I subtype. The NE-A subtype, with a large fraction of patients having low Teff and low TAM, as well as the most patients with low Teff and high TAM, had a mOS (11.56 months) with atezolizumab treatment, which was similar to the BEP (11.37 months).

The balance of TAMs and CD8+ T effector cells (T-eff) delineated the response outcome, where low TAM predicted a longer OS. The four NMF subtypes contained patient subgroups with different ratios of TAMs and T-eff, which may point to a potential tumor-intrinsic control immune compartment within SCLC. It was previously reported that the myeloid compartment in SCLC shows an increase in mononuclear cells (monocytes/macrophages) with an immunosuppressive phenotype, similar to the macrophages associated with idiopathic pulmonary fibrosis (IPF) (Chan et al. Cancer Cell. 39:1479-1496 (2021)). Chan and colleagues showed that CD8+ T cells in the TME of the PLCG2+ SCLC subpopulation may be impeded by the immunosuppressive monocytes and macrophage cells (Chan et al. Cancer Cell. 39:1479-1496 (2021)). Without being bound by theory, the same could be true in the nNE-I subtype, which was the only subtype with a large portion of patients whose tumors were PLCG2+, as well as the largest group of patients with both high TAM and high T-eff levels. Therefore, even though nNE-I has a high level of CD8+T-effector cells, these could be impeded by the high level of inhibitory TAMs.

Although the PLCG2+ SCLC cluster may represent only a small fraction of the malignant cells in the tumors under study, this small subpopulation was highly correlated with poor survival in previous studies (Chan et al. Cancer Cell. 39:1479-1496 (2021)). The elevated PLCG2 gene expression observed in the nNE-I subtype compared with the other identified subtypes, needs further characterization by IHC to validate the finding. The association with PLCG2+ tumor cells and fibrotic macrophages is in agreement with association of the nNE-I subtype, PLCG2 gene expression, and TAM infiltration observed in the present study. The nNE-I subtype had the shortest OS and PFS, whereas the NE-I subtype, which contained no patients whose tumors expressed PLCG2+, had the longest OS and a PFS similar to the BEP. When considering therapies for the nNE-I and NE-I subtypes, the nNE-I subtype might benefit from myeloid repolarization agents. In myeloid cell repolarization, TAMs and myeloid-derived suppressor cells (MDSCs) are reprogrammed from an immunosuppressive to pro-inflammatory phenotype using a Toll-like receptor 7 (TLR7) agonist, such as folate-targeted TLR7 agonist (FA-TLR7-1A), to specifically reactivate TAMs and MDSCs (Cresswell et al. Cancer Res. 81:671-684 (2021); Luo et al. Front Immunol. 13:816761 (2022)).

Since REST expression was higher in the nNE-I subtype than in the other NMF subtypes, another possibility as a potential therapeutic strategy for the nNE-I subtype would be to focus a therapy on REST. REST is a tumor suppressor gene that functions as the transcriptional repressor of neuronal genes in non-neuronal cells to restrict the expression of neuronal genes to the nervous system. In SCLC tumor cells, an SCLC-specific isoform of REST (sREST) is highly expressed, whereas REST expression is undetectable, suggesting that the expression of sREST correlates with the pathogenesis of SCLC. It was shown in NCI-N417 cells that overexpression of REST caused repression of sREST, leading to tumor suppression in SCLC cells. The NE-I subtype had a larger proportion of patients with high T-eff and low TAM levels compared with other NMF subtypes. Therefore, patients in this group may be responsive to anti-CTLA-4 immunotherapy (Antonia et al. Lancet Oncol. 17:883-895 (2016)). It is thought that the blockade of CTLA-4 most likely impacts the stage of Tcell activation in the draining lymph nodes where CTLA-4 expressing T regulatory cells (Tregs) remove CD80/CD86 from the surface of antigen-presenting cells, thereby reducing their ability to effectively stimulate tumor-specific T cells (Downey et al. Clin Cancer Res. 13:6681-6688 (2007); Ribas et al. J Clin Oncol. 23:8968-8977 (2005)). Reducing the activity of immune inhibiting Tregs should especially benefit the subgroup of NE-I patients who have high levels of immune-activating Teff and low levels of immune inhibitory TAM in their tumor.

In conclusion, from this study, an image of SCLC emerges where different subtypes enlist divergent gene programs causing heterogeneity, and in the case of the nNE-I subtype, leads to an immunosuppressed TME. Two inflamed subtypes were identified with distinct clinical outcomes dependent on the balance of T-effector to TAM infiltration. These results have the potential to lead to more targeted therapies and immunotherapeutic approaches in the future for SCLC patients based on molecular subtypes.

### OTHER EMBODIMENTS

Although the foregoing invention has been described in some detail by way of illustration and example for purposes of clarity of understanding, the descriptions and examples should not be construed as limiting the scope of the invention.

What is claimed is:

1. A method of classifying a small cell lung cancer (SCLC) in a human patient, the method comprising:
(a) assaying mRNA in a tumor sample from the patient to provide a transcriptional profile of the patient's tumor; and
(b) assigning the patient's tumor sample into one of the following four subtypes based on the transcriptional profile of the patient's tumor: neuroendocrine inflamed (NE-I), neuroendocrine NEUROD-driven (NE-N), neuroendocrine achaete-scute homolog 1 (ASCL1)-driven (NE-A), or non-neuroendocrine inflamed (nNE-I),
thereby classifying the SCLC in the patient.

2. A method of treating an SCLC in a human patient, the method comprising:
classifying the SCLC in the patient according to the method of claim 1; and
administering an anti-cancer therapy to the patient based on the SCLC subtype.

3. The method of claim 2, wherein the anti-cancer therapy comprises atezolizumab.

4. The method of claim 2, wherein
(a) assaying mRNA in the tumor sample from the patient comprises RNA sequencing (RNA-seq), quantitative PCR (qPCR), reverse transcription-quantitative polymerase chain reaction (RT-qPCR), multiplex qPCR or RT-qPCR, microarray analysis, serial analysis of gene expression (SAGE), MASSARRAY® technique, in situ hybridization (ISH), or a combination thereof;
(b) the tumor sample is a formalin-fixed and paraffin-embedded (FFPE) sample, an archival sample, a fresh sample, or a frozen sample;
(c) the tumor sample is a pre-treatment tumor sample;
(d) the method further comprises administering an additional therapeutic agent to the patient, wherein the additional agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof.

5. (canceled)

6. The method of claim 2, wherein the four subtypes are identified by non-negative matrix factorization (NMF) based on a set of 5829 genes as set forth in Table 1.

7. (canceled)

8. The method of claim 2, wherein the method further comprises determining the mRNA expression level of one or more of the following gene signatures in the tumor sample from the patient:
(a) a neuroendocrine (NE) signature comprising CHGA, DLL3, NEUROD1, INSM1, and ASCL1;
(b) a non-NE signature comprising YAP1, POU2F3, MYC, and REST;
(c) an endothelial-mesenchymal transition (EMT) signature comprising ZEB1, ZEB2, SNAI1, and TWIST1;
(d) a T-effector (T-eff) signature comprising CD8A, GZBA, GZMB, PRF1, IFNG, CXCL9, CXCL10, and TBX21;
(e) a B/plasma cell (B/PC) signature comprising CD79A, MS4A1, MZB1, and JCHAIN;
(f) an antigen-presenting machinery (APM) signature comprising TAP1, TAP2, B2M, HLA-A, and HLA-C;
(g) a checkpoint signature comprising PDCD1, CD274, LAG3, CTLA4, BTLA, and TIGIT;
(h) an immune stimulatory signature comprising CD27, CD28, CD40, CD40LG, IL2RB, TNFRSF4, TNFSF4, ICOSLG, ICOS, TNFRSF18, TNFSF18, TNFRSF9, and TNFSF9;
(i) an immune inhibitory signature comprising CD274, PDCD1, PDCDILG2, CTLA4, CD86, CD80, CD200, CD200R1, VSIR, IGSF11, LAG3, CLEC4G, BTLA, CD160, TNFRSF14, HAVCR2, CEACAM1, HMGB1, LGALS9, and TIGIT;
(j) a general myeloid signature comprising CLEC9A, LAMP3, CD68, MRC1, TGM2, NOS2, SOCS3, CD163, FCGR3A, and FCGR3B;
(k) an angiogenesis signature comprising VEGFA, KDR, ESM1, PECAM1, ANGPTL4, and CD34;
(1) a tumor-associated macrophage signature comprising MARCO, ACP5, VSIG4, MRC1, MSR1, MCEMP1, CYP27A1, OLR1, GRN, GLIPR2, ARRDC4, CIQC, APOE, FOLR2, CTSD, and SPP1;
(m) a ciliated cell signature comprising C9orf24 and C20orf85;
(n) a basal cell signature comprising TP63, KRT15, and KRT17; and/or
(o) a goblet cell signature comprising SLC5A5 and SAA1.

9. The method of claim 8, wherein the patient's tumor sample is assigned into the NE-I subtype, and the patient's tumor sample has an increased expression level, relative to a reference expression level, of the neuroendocrine signature, the T-eff signature, the B/PC signature, the checkpoint signature, the APM signature, the immune stimulatory signature, the immune inhibitory signature, the general myeloid signature, the ciliated cell signature, the basal cell signature, and/or the goblet cell signature.

10. The method of claim 8, wherein the patient's tumor sample is assigned into the nNE-I subtype, and the patient's tumor sample has an increased expression level, relative to a reference expression level, of the non-neuroendocrine signature, the T-eff signature, the B/PC signature, the checkpoint signature, the APM signature, the immune stimulatory signature, the immune inhibitory signature, the general myeloid signature, and/or the tumor-associated macrophage signature.

11. (canceled)

12. The method of claim 2, wherein the patient's tumor sample is assigned into the NE-I subtype, and the patient's tumor sample has:
(i) an increased expression level, relative to a reference expression level, of ASCL1 or YAP1;
(ii) an increased expression level, relative to a reference expression level, of the TGF beta signaling, p53 pathway, EMT, and/or NOTCH signaling MSigDB hallmark signatures;
(iii) a decreased expression level, relative to a reference expression level, of the MYC targets MSigDB hallmark signature; and/or
(iv) an increased expression level, relative to a reference expression level, of PD-L1 in tumor-infiltrating immune cells.

13. (canceled)

14. The method of claim 2, wherein the patient's tumor sample is assigned into the NE-I subtype, and the patient's tumor sample has:
(i) an increased expression level, relative to a reference expression level, of a T-eff signature comprising CD8A, GZBA, GZMB, PRF1, IFNG, CXCL9, CXCL10, and TBX21; and
(ii) a decreased expression level, relative to a reference expression level, of a tumor-associated macrophage (TAM) signature comprising MARCO, ACP5, VSIG4, MRC1, MSR1, MCEMP1, CYP27A1, OLR1, GRN, GLIPR2, ARRDC4, CIQC, APOE, FOLR2, CTSD, and SPP1.

15. The method of claim 2, wherein the patient's tumor sample is assigned into the NE-I subtype, and the patient's tumor sample has an elevated expression level, relative to a reference expression level, of a ciliated cell signature comprising C9orf24 and C20orf85, a basal cell signature comprising TP63, KRT15, and KRT17, and/or a goblet cell signature comprising SLC5A5 and SAA1.

16. (canceled)

17. The method of claim 2, wherein the patient's tumor sample is assigned into the nNE-I subtype, and the patient's tumor sample has:
(i) an increased expression level, relative to a reference expression level, of ASCL1, YAP1, POU2F3, REST, and/or MYC;
(ii) an increased expression level, relative to a reference expression level, of the MYC targets MSigDB hallmark signature;
(iii) a decreased expression level, relative to a reference expression level, of the G2M checkpoint, SHH signaling, mitotic spindle, spermatogenesis, and/or pancreas beta cells MSigDB hallmark signatures; and/or
(iv) an increased expression level, relative to a reference expression level, of PD-L1 in tumor-infiltrating immune cells.

18. (canceled)

19. The method of claim 2, wherein the patient's tumor sample is assigned into the nNE-I subtype, and the patient's tumor sample has:
(i) an increased expression level, relative to a reference expression level, of a T-eff signature comprising CD8A, GZBA, GZMB, PRF1, IFNG, CXCL9, CXCL10, and TBX21; and
(ii) an increased expression level, relative to a reference expression level, of a TAM signature comprising MARCO, ACP5, VSIG4, MRC1, MSR1, MCEMP1, CYP27A1, OLR1, GRN, GLIPR2, ARRDC4, C1QC, APOE, FOLR2, CTSD, and SPP1.

20. (canceled)

21. The method of claim 2, wherein the patient's tumor sample is assigned into the NE-A subtype, and the patient's tumor sample has:
(i) an increased expression level, relative to a reference expression level, of ASCL1; and/or
(ii) a decreased expression level, relative to a reference expression level, of TGF beta signaling, p53 pathway, EMT, NOTCH signaling, MYC targets, and/or WNT signaling MSigDB hallmark signatures.

22. (canceled)

23. The method of claim 2, wherein the patient's tumor sample is assigned into the NE-N subtype, and the patient's tumor sample has:
(i) an increased expression level, relative to a reference expression level, of NEUROD1; and/or
(ii) an increased expression level, relative to a reference expression level, of the DNA repair, MYC targets, WNT signaling, G2M checkpoint, SHH signaling, mitotic spindle, or spermatogenesis MSigDB hallmark signatures.

24.-28. (canceled)

29. The method of claim 2, wherein the patient's tumor sample is assigned into the NE-I subtype, and the method further comprises treating the patient by administering an anti-cancer therapy comprising atezolizumab or a CTLA4 antagonist antibody to the patient.

30. (canceled)

31. (canceled)

32. A method of identifying a patient having an SCLC who is likely to benefit from an anti-cancer therapy comprising atezolizumab, the method comprising: determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, CIQC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising atezolizumab.

33. A method of selecting a therapy for a patient having an SCLC, the method comprising:
(a) determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, CIQC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising atezolizumab; and
(b) selecting an anti-cancer therapy comprising atezolizumab for the patient identified as one who is likely to benefit from the anti-cancer therapy.

34. A method of treating a patient having an SCLC, the method comprising:
(a) determining the expression level of a T-eff signature comprising CD8A, GZBA, GZMB, PRF1, IFNG, CXCL9, CXCL10, and TBX21 and the expression level of a TAM signature comprising MARCO, ACP5, VSIG4, MRC1, MSR1, MCEMP1, CYP27A1, OLR1, GRN, GLIPR2, ARRDC4, CIQC, APOE, FOLR2, CTSD, and SPP1 in a tumor sample from the patient, wherein an increased expression level of the T-eff signature relative to a reference expression level and a decreased expression level of the TAM signature relative to a reference expression level identifies the patient as one who is likely to benefit from an anti-cancer therapy comprising atezolizumab; and
(b) administering an anti-cancer therapy comprising atezolizumab to the patient identified as one who is likely to benefit from the anti-cancer therapy.

35.-37. (canceled)

38. The method of claim 2, wherein the patient's tumor sample is assigned into the NE-A subtype or the NE-N subtype, and the method further comprises treating the patient by administering to the patient a DNA damage response (DDR)-targeting agent, wherein the DDR-targeting agent is an anti-delta-like ligand 3 (DLL3) antibody-drug conjugate (ADC) or an anti-DLL3 bispecific T cell engager (BiTE).

39. (canceled)

40. The method of claim 2, wherein the patient's tumor sample is assigned into the nNE-I subtype, and the method further comprises treating the patient by administering to the patient a myeloid repolarization agent or a REST-targeted therapy.

41.-46. (canceled)

47. The method of claim 3, wherein the anti-cancer therapy comprising atezolizumab further comprises carboplatin and etoposide.

48. The method of claim 47, wherein the anti-cancer therapy is administered to the patient in a dosing regimen comprising:
(i) an induction phase comprising four 21-day cycles, wherein atezolizumab is administered to the patient at a dose of 1200 mg intravenously (IV) on Day 1 of each cycle, carboplatin is administered to the patient at an initial target area under the curve (AUC) of 5 mg/mL/min IV on Day 1 of each cycle, and etoposide is administered to the patient at a dose of 100 mg/m2 IV on Days 1, 2, and 3 of each cycle; and
(ii) a maintenance phase comprising one or more 21-day cycles, wherein atezolizumab is administered to the patient at a dose of 1200 mg IV on Day 1 of each 21-day cycle.

49.-53. (canceled)

54. A kit for performing the method of claim 2.

55.-63. (canceled)


SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR